(19) United States
(12) Patent Application Publication
(10) Pub. No.: US 2020/0046854 A1
(43) Pub. Date: Feb. 13, 2020

(54) PREVENTION OF MUSCULAR DYSTROPHY BY CRISPR/CPF1-MEDIATED GENE EDITING

(71) Applicant: The Board of Regents of the University of Texas System, Austin, TX (US)

(72) Inventors: Yu ZHANG, Dallas, TX (US); Chengzu LONG, New York, NY (US); Rhonda BASSEL-DUBY, Dallas, TX (US); Eric OLSON, University Park, TX (US)

(73) Assignee: The Board of Regents of the University of Texas System, Austin, TX (US)

(21) Appl. No.: 16/464,124

(22) PCT Filed: Nov. 28, 2017
(86) PCT No.: PCT/US2017/063468

§ 371 Date: May 24, 2019

Related U.S. Application Data
(60) Provisional application No. 62/426,853, filed on Nov. 28, 2016.

(51) Int. Cl. A61K 48/00; C12N 9/22; C12N 15/11; C12N 15/86; C12N 7/00; C12N 5/0735; C12N 5/074; A61K 9/00; A61P 21/00

(52) U.S. Cl. CPC A61K 48/0075; C12N 9/22; C12N 15/11; C12N 15/86; C12N 7/00; C12N 5/0606; C12N 2830/50; A61K 9/0019; A61P 21/00; C12N 2800/80; C12N 2310/20; C12N 2750/14143; C12N 5/0696

(57) ABSTRACT

Duchenne muscular dystrophy (DMD) is an inherited X-linked disease caused by mutations in the gene encoding dystrophin, a protein required for muscle fiber integrity. The disclosure reports CRISPR/Cpf1-mediated gene editing (Myo-editing) is effective at correcting the dystrophin gene mutation in the mdx mice, a model for DMD. Further, the disclosure reports optimization of germline editing of mdx mice by engineering the permanent skipping of mutant exon and extending exon skipping to also correct the disease by post-natal delivery of adeno-associated virus (AAV). AAV-mediated Myo-editing can efficiently rescue the reading frame of dystrophin in mdx mice in vivo. The disclosure reports means of Myo-editing-mediated exon skipping has been successfully advanced from somatic tissues in mice to human DMD patients-derived iPSCs (induced pluripotent stem cells). Custom Myo-editing was performed on iPSCs from patients with differing mutations and successfully restored dystrophin protein expression for all mutations in iPSCs-derived cardiomyocytes.

### PRIORITY CLAIM

This application is a national phase application under 35 U.S.C. § 371 of International Application No. PCT/US2017/063468, filed Nov. 28, 2017, which claims priority to U.S. Provisional Patent Application Ser. No. 62/426,853 which was filed on Nov. 28, 2016, and entitled “Prevention of Muscular Dystrophy by CRISPR/Cpf1-Mediated Gene Editing,” the disclosure of each of which is hereby incorporated by reference in its entirety.

### FEDERAL FUNDING SUPPORT CLAUSE

This invention was made with government support under DK-099653 and U54-HD 087351 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 16, 2019, is named UTSDP3124US.txt and is 1,135 KB in size.

### FIELD OF THE DISCLOSURE

The present disclosure relates to the fields of molecular biology, medicine and genetics. More particularly, the disclosure relates to the use of genome editing to treat Duchenne muscular dystrophy (DMD).

### BACKGROUND

Duchenne muscular dystrophy (DMD) is an X-linked recessive disease caused by mutations in the gene coding for dystrophin, which is a large cytoskeletal protein essential for integrity of muscle cell membranes. DMD causes progressive muscle weakness, culminating in premature death by the age of 30, generally from cardiomyopathy. There is no effective treatment for this disease. Numerous approaches to rescue dystrophin expression in DMD have been attempted, including delivery of truncated dystrophin or utrophin by recombinant adeno-associated virus (rAAV) and skipping of mutant exons with anti-sense oligonucleotides and small molecules. However, these approaches cannot correct DMD mutations or permanently restore dystrophin expression. Accordingly, there is a need in the art for compositions and methods for treating DMD that correct DMD mutations to address the underlying cause of the disease, thereby permanently restore dystrophin expression.

### SUMMARY

The disclosure provides a composition comprising a sequence encoding a Cpf1 polypeptide and a sequence encoding a DMD guide RNA (gRNA), wherein the DMD gRNA targets a dystrophin splice site, and wherein the DMD gRNA comprises any one of SEQ ID No. 448 to 770. In some embodiments, the sequence encoding the Cpf1 polypeptide is isolated or derived from a sequence encoding a Lachnospiraceae Cpf1 polypeptide. In some embodiments, the sequence encoding the Cpf1 polypeptide is isolated or derived from a sequence encoding an Acidaminococcus Cpf1 polypeptide. In some embodiments, the sequence encoding the Cpf1 polypeptide or the sequence encoding the DMD gRNA comprises an RNA sequence. In some embodiments, the RNA sequence is an mRNA sequence. In some embodiments, the RNA sequence comprises at least one chemically-modified nucleotide. In some embodiments, the sequence encoding the Cpf1 polypeptide comprises a DNA sequence.

In some embodiments, a first vector comprises the sequence encoding the Cpf1 polypeptide and a second vector comprises the sequence encoding the DMD gRNA. In some embodiments, the first vector or the sequence encoding the Cpf1 polypeptide further comprises a first polyA sequence. In some embodiments, the second vector or the sequence encoding the DMD gRNA further comprises a second polyA sequence. In some embodiments, the first vector or the second vector further comprises a sequence encoding a detectable marker. In some embodiments, the detectable marker is a fluorescent maker.

In some embodiments, the first vector or the sequence encoding the Cpf1 polypeptide further comprises a first promoter sequence. In some embodiments, the second vector or the sequence encoding the DMD gRNA further comprises a second promoter sequence. In some embodiments, the promoter first promoter sequence and the second promoter sequence are identical. In some embodiments, the first promoter sequence and the second promoter sequence are not identical. In some embodiments, the first promoter sequence or the second promoter sequence comprises a constitutive promoter. In some embodiments, the first promoter sequence or the second promoter sequence comprises an inducible promoter. In some embodiments, the first promoter sequence or the second promoter sequence comprises a muscle-cell specific promoter. In some embodiments, the muscle-cell specific promoter is a myosin light chain-2 promoter, an α-actin promoter, a troponin 1 promoter, a Na⁺/Ca²⁺ exchanger promoter, a dystrophin promoter, an α7 integrin promoter, a brain natriuretic peptide promoter, an αB-crystallin/small heat shock protein promoter, an a-myosin heavy chain promoter, or an ANF promoter.

In some embodiments, the first vector or the second vector further comprises a sequence encoding 2A-like self-cleaving domain. In some embodiments, the sequence encoding 2A-like self-cleaving domain comprises a TaV-2A peptide.

In some embodiments, the vector comprises the sequence encoding the Cpf1 polypeptide and the sequence encoding the DMD gRNA. In embodiments, the vector further comprises a polyA sequence. In embodiments, the vector further comprises a promoter sequence. In embodiments, the promoter sequence comprises a constitutive promoter. In further embodiments, the promoter sequence comprises an inducible promoter. In embodiments, the promoter sequence comprises a muscle-cell specific promoter. In some embodiments, the muscle-cell specific promoter is a myosin light chain-2 promoter, an a-actin promoter, a troponin 1 promoter, a Na⁺/Ca²⁺ exchanger promoter, a dystrophin promoter, an α7 integrin promoter, a brain natriuretic peptide promoter, an αB-crystallin/small heat shock protein promoter, an α-myosin heavy chain promoter, or an ANF promoter.

In embodiments, the composition comprises a sequence codon optimized for expression in a mammalian cell. In further embodiments, the composition comprises a sequence codon optimized for expression in a human cell. In embodiments, the sequence encoding the Cpf1 polypeptide is codon optimized for expression in human cells.

In some embodiments, the splice site is a splice donor site. In some embodiments, the splice site is a splice acceptor site.

In further embodiments, the first vector or the second vector is a non-viral vector. In embodiments, the non-viral vector is a plasmid. In embodiments, a liposome or a nanoparticle comprises the first vector or the second vector.

In embodiments, the first vector or the second vector is a viral vector. In embodiments, the viral vector is an adeno-associated viral (AAV) vector. In embodiments, the AAV vector is replication-defector or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

In some embodiments, the composition further comprises a single-stranded DMD oligonucleotide. In some embodiments, the composition further comprises a pharmaceutically acceptable carrier.

Also provided is a cell comprising a composition of the disclosure. In embodiments, the cell is a muscle cell, a satellite cell or a precursor thereof. In some embodiments, the cell is an iPSC or an iCM.

Also provided is a composition comprising a cell of the instant disclosure.

Also provided is a method of correcting a dystrophin gene defect comprising contacting a cell and a composition of the disclosure under conditions suitable for expression of the Cpf1 polypeptide and the gRNA, wherein the Cpf1 polypeptide disrupts the dystrophin splice site; and wherein disruption of the splice site results in selective skipping of a mutant DMD exon. In some embodiments, the mutant DMD exon is exon 23. In some embodiments, the mutant DMD exon is exon 51. In embodiments, the cell is in vivo, ex vivo, in vitro or in situ.

The disclosure also provides a of treating muscular dystrophy in a subject in need thereof comprising administering to the subject a therapeutically effective amount of a composition according to the instant disclosure. In embodiments, the composition is administered locally. In embodiments, the composition is administered directly to a muscle tissue. In embodiments, the composition is administered by intramuscular infusion or injection. In embodiments, the muscle tissue comprises a tibialis anterior tissue, a quadricep tissue, a soleus tissue, a diaphragm tissue or a heart tissue. In some embodiments, the composition is administered systemically. In some embodiments, the composition is administered by intravenous infusion or injection.

In embodiments, following administration of the composition, the subject exhibits normal dystrophin-positive myofibers, and mosaic dystrophin-positive myofibers containing centralized nuclei, or a combination thereof. In some embodiments, following administration of the composition, the subject exhibits an emergence or an increase in a level of abundance of normal dystrophin-positive myofibers when compared to an absence or a level of abundance of normal dystrophin-positive myofibers prior to administration of the composition. In some embodiments, following administration of the composition, the subject exhibits an emergence or an increase in a level of abundance of mosaic dystrophin-positive myofibers containing centralized nuclei when compared to an absence or an level of abundance of mosaic dystrophin-positive myofibers containing centralized nuclei prior to administration of the composition. In some embodiments, the subject exhibits a decreased serum CK level when compared to a serum CK level prior to administration of the composition. In embodiments, following administration of the composition, the subject exhibits improved grip strength when compared to a grip strength prior to administration of the composition.

In some embodiments, the method comprises administering a therapeutically effective amount of a composition disclosed herein, wherein the cell is autologous. In some embodiments, the method comprises administering a therapeutically effective amount of the composition, wherein the cell is allogeneic.

In embodiments, the subject is a neonate, an infant, a child, a young adult, or an adult. In embodiments, the subject has muscular dystrophy. In embodiments, the subject is a genetic carrier for muscular dystrophy. In some embodiments, the subject is male. In some embodiments, the subject is female. In some embodiments, the subject appears to be asymptomatic and wherein a genetic diagnosis reveals a mutation in one or both copies of a DMD gene that impairs function of the DMD gene product. In some embodiments, the subject presents an early sign or symptom of muscular dystrophy. In some embodiments, the early sign or symptom of muscular dystrophy comprises loss of muscle mass or proximal muscle weakness. In embodiments, the loss of muscle mass or proximal muscle weakness occurs in one or both leg(s) and/or a pelvis, followed by one or more upper body muscle(s). In embodiments, the early sign or symptom of muscular dystrophy further comprises pseudohypertrophy, low endurance, difficulty standing, difficulty walking, and/or difficulty ascending a staircase or a combination thereof. In embodiments, the subject presents a progressive sign or symptom of muscular dystrophy. In embodiments, the progressive sign or symptom of muscular dystrophy comprises muscle tissue wasting, replacement of muscle tissue with fat, or replacement of muscle tissue with fibrotic tissue. In embodiments, the subject presents a later sign or symptom of muscular dystrophy. In some embodiments, the later sign or symptom of muscular dystrophy comprises abnormal bone development, curvature of the spine, loss of movement, and paralysis. In embodiments, the subject presents a neurological sign or symptom of muscular dystrophy. In embodiments, the neurological sign or symptom of muscular dystrophy comprises intellectual impairment and paralysis. In embodiments, administration of the composition occurs prior to the subject presenting one or more progressive, later or neurological signs or symptoms of muscular dystrophy. In some embodiments, the subject is less than 10 years old. In some embodiments, the subject is less than 5 years old. In some embodiments, the subject is less than 2 years old.

The disclosure also provides a use of a therapeutically-effective amount of a composition for treating muscular dystrophy in a subject in need thereof.

It is contemplated that any method or composition described herein can be implemented with respect to any other method or composition described herein.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. Correction of DMD mutations by Cpf1-mediated genome editing. (FIG. 1A) A DMD deletion of exons 48-50 results in splicing of exon 47 to 51, generating an out-of-frame mutation of dystrophin. Two strategies were used for the restoration of dystrophin expression by Cpf1. In the “reframing” strategy, small INDELs in exon 51 restore the protein reading frame of dystrophin. The “exon skipping” strategy is achieved by disruption of the splice acceptor of exon 51, which results in splicing of exon 47 to 52 and restoration of the protein reading frame. (FIG. 1B) The 3′ end of an intron is T-rich, which generates Cpf1 PAM sequences enabling genome cleavage by Cpf1. (FIG. 1C) Illustration of Cpf1 gRNA targeting DMD exon 51. The T-rich PAM (red line) is located upstream of exon 51 near the splice acceptor site. The sequence of the Cpf1 g1 gRNA targeting exon 51 is shown, highlighting the complementary nucleotides in blue. Cpf1 cleavage produces a staggered-end distal to the PAM site (demarcated by red arrowheads). The 5′ region of exon 51 is shaded in light blue. Exon sequence is upper case. Intron sequence is lower case. (FIG. 1D) Illustration of a plasmid encoding human codon-optimized Cpf1 (hCpf1) with a nuclear localization signal (NLS) and 2A-GFP. The plasmid also encodes a Cpf1 gRNA driven by the U6 promoter. Cells transfected with this plasmid express GFP, allowing for selection of Cpf1-expressing cells by FACS. (FIG. 1E) T7E1 assays using human 293T cells or DMD iPSCs (RIKEN51) transfected with plasmid expressing LbCpf1 or AsCpf1, gRNA and GFP show genome cleavage at DMD exon 51. Red arrowheads point to cleavage products. M, marker.

FIGS. 2A-I. DMD iPSC-derived cardiomyocytes express dystrophin after Cpf1-mediated genome editing by reframing. (FIG. 2A) DMD skin fibroblast-derived iPSCs were edited by Cpf1 using gRNA (corrected DMD-iPSCs) and then differentiated into cardiomyocytes (corrected cardiomyocytes) for analysis of genetic correction of the DMD mutation. (FIG. 2B) A DMD deletion of exons 48-50 results in splicing of exon 47 to 51, generating an out-of-frame mutation of dystrophin. Forward primer (F) targeting exon 47 and reverse primer (R) targeting exon 52 were used in RT-PCR to confirm the reframing strategy by Cpf1-meditated genome editing in cardiomyocytes. Uncorrected cardiomyocytes lack exons 48-50. In contrast, after reframing, exon 51 is placed back in-frame with exon 47. (FIG. 2C) Sequencing of representative RT-PCR products shows that uncorrected DMD iPSC-derived cardiomyocytes have a premature stop codon in exon 51, which creates a nonsense mutation. After Cpf1-mediated reframing, the ORF of dystrophin is restored. Dashed red line denotes exon boundary. (FIG. 2D) Western blot analysis shows dystrophin expression in a mixture of DMD iPSC-derived cardiomyocytes edited by reframing with LbCpf1 or AsLpf1 and g1 gRNA. Even without clonal selection, Cpf1-mediated reframing is efficient and sufficient to restore dystrophin expression in the cardiomyocyte mixture. αMHC is loading control. (FIG. 2E) Immunocytochemistry shows dystrophin expression in iPSC-derived cardiomyocyte (CM) mixtures following LbCpf1- or AsCpf1-mediated reframing. Dystrophin staining (red); Troponin I staining (green). Scale bar=100 microns. (FIG. 2F) Western blot analysis shows dystrophin expression in single clones (#2 and #5) of iPSC-derived cardiomyocytes following clonal selection after LbCpf1-mediated reframing. αMHC is loading control. (FIG. 2G) Immunocytochemistry showing dystrophin expression in clone #2 LbCpf1-edited iPSC-derived cardiomyocytes. Scale bar=100 microns. (FIG. 2H) Quantification of mitochondrial DNA copy number in single clones (#2 and #5) of LbCpf1-edited iPSC-derived cardiomyocytes. Data are represented as mean±SEM (n=3). (&) P<0.01; (#) P<0.005; (ns) not significant. (FIG. 2I) Basal oxygen consumption rate (OCR) of single clones (#2 and #5) of LbCpf1-edited iPSC-derived cardiomyocytes, and OCR in response to oligomycin, FCCP, and Rotenone and Antimycin A, normalized to cell number (order left to right for each test is the same as FIG. 2H). Data are represented as mean±SEM (n=5). (*) P<0.05; (&) P<0.01; (#) P<0.005; (ns) not significant.

FIGS. 3A-H. DMD iPSC-derived cardiomyocytes express dystrophin after Cpf1-mediated exon skipping. (FIG. 3A) Two gRNAs, either gRNA (g2 or g3), which target intron 50, and the other (g1), which targets exon 51, were used to direct Cpf1-mediated removal of the exon 51 splice acceptor site. (FIG. 3B) T7E1 assay using 293T cells transfected with LbCpf1 and gRNA2 (g2) or gRNA3 (g3) shows cleavage of the DMD locus at intron 50. Red arrowheads denote cleavage products. M, marker. (FIG. 3C) PCR products of genomic DNA isolated from DMD-iPSCs transfected with a plasmid expressing LbCpf1, g1+g2 and GFP. The lower band (red arrowhead) indicates removal of the exon 51 splice acceptor site. (FIG. 3D) Sequence of the lower PCR band from panel c shows a 200-bp deletion, spanning from the 3′-end of intron 50 to the 5′-end of exon 51. This confirms removal of the “ag” splice acceptor of exon 51. The sequence of the uncorrected allele is shown above that of the LbCpf1-edited allele. (FIG. 3E) RT-PCR of iPSC-derived cardiomyocytes using primer sets described in FIG. 2B. The 700-bp band in the WT lane is the dystrophin transcript from exon 47-52; the 300-bp band in the uncorrected lane is the dystrophin transcript from exon 47-52 with exon 48-50 deletion; and the lower band in the g1+g2 mixture lane (edited by LbCpf1) shows exon 51 skipping. (FIG. 3F) Sequence of the lower band from panel e (g1+g2 mixture lane) confirms skipping of exon 51, which reframed the DMD ORF. (FIG. 3G) Western blot analysis shows dystrophin protein expression in iPSC-derived cardiomyocyte mixtures after exon 51 skipping by LbCpf1 with g1+g2. αMHC is loading control. (FIG. 3H) Immunocytochemistry shows dystrophin expression in iPSC-derived cardiomyocyte mixtures (CMs) following Cpf1-mediated exon skipping with g1+g2 gRNA compared to WT and uncorrected CMs. Dystrophin staining (red). Troponin I staining (green). Scale bar=100 microns.

FIGS. 4A-D. CRISPR-Cpf1-mediated editing of exon 23 of the mouse DMD gene. (FIG. 4A) Illustration of mouse Dmd locus highlighting the mutation at exon 23. Sequence shows the nonsense mutation caused by C to T transition, which creates a premature stop codon. (FIG. 4B) Illustration showing the targeting location of gRNAs (g1, g2 and g3) (shown in light blue) on exon 23 of the Dmd gene. Red line represents LbCpf1 PAM. (FIG. 4C) T7E1 assay using mouse 10T1/2 cells transfected with LbCpf1 or AsCpf1 with different gRNAs (g1, g2 or g3) targeting exon 23 shows that LbCpf1 and AsCpf1 have different cleavage efficiency at the Dmd exon 23 locus. Red arrowheads show cleavage products of genome editing. M, marker. (FIG. 4D) Illustration of LbCpf1-mediated gRNA (g2) targeting of Dmd exon 23. Red arrowheads indicate the cleavage site. The ssODN HDR template contains the mdx correction, four silent mutations (green) and a TseI restriction site (underlined).

FIGS. 5A-F. CRISPR-LbCpf1-mediated Dmd correction in mdx mice. (FIG. 5A) Strategy of gene correction in mdx mice by LbCpf1-mediated germline editing. Zygotes from intercrosses of mdx parents were injected with gene editing components (LbCpf1 mRNA, g2 gRNA and ssODN) and reimplanted into pseudo-pregnant mothers, which gave rise to pups with gene correction (mdx-C). (FIG. 5B) Illustration showing LbCpf1 correction of mdx allele by HDR or NHEJ. (FIG. 5C) Genotyping results of LbCpf1-edited mdx mice. Top panel shows T7E1 assay. Blue arrowhead denotes uncleaved DNA and red arrowhead shows T7E1 cleaved DNA. Bottom panel shows TseI RFLP assay. Blue arrowhead denotes uncorrected DNA. Red arrowhead points to TseI cleavage indicating HDR correction. mdx-C1-C5 denotes LbCpf1-edited mdx mice. (FIG. 5D) Top panel shows sequence of WT Dmd exon 23. Middle panel shows sequence of mdx Dmd exon 23 with C to T mutation, which generates a STOP codon. Bottom panel shows sequence of Dmd exon 23 with HDR correction by LbCpf1-mediated editing. Black arrow points to silent mutations introduced by the ssODN HDR template. (FIG. 5E) H&E of tibialis anterior (TA) and gastrocnemius/plantaris (G/P) muscles from WT, mdx and LbCpf1-edited mice (mdx-C). (FIG. 5F) Immunohistochemistry of TA and G/P muscles from WT, mdx and LbCpf1-edited mice (mdx-C) using antibody to dystrophin (red). mdx muscle showed fibrosis and inflammatory infiltration, whereas mdx-C muscle showed normal muscle structure.

FIGS. 6A-C. Genome editing at DMD exon 51 by LbCpf1 or AsCpf1. (FIG. 6A) DNA sequencing of DMD exon 51 from a mixture of DMD patient (RIKEN 51) skin fibroblast-derived iPSCs edited by LbCpf1 or AsCpf1 using g1. Sequences of individual edited DMD allele are shown beneath the uncorrected DMD allele. Δ denotes nucleotide deletion. (FIG. 6B) DNA sequencing of DMD exon 51 from a single clone of DMD patient skin fibroblast-derived iPSCs edited by LbCpf1 or AsCpf1 using g1. (FIG. 6C) DNA Sequencing of PCR products of 10T1/2 cells following LbCpf1-editing with g2 or g3. WT sequence is on top and INDEL sequences are on the bottom.

FIGS. 7A-B. Histological analysis of muscles from WT, mdx and LbCpf1-edited mice (mdx-C). (FIG. 7A) Immunohistochemistry and H&E staining of whole tibialis anterior (TA) muscle. Dystrophin staining is red. (FIG. 7B) Immunohistochemistry and H&E staining of whole gastrocnemius/plantaris (G/P) muscles. Dystrophin staining is red.

### DETAILED DESCRIPTION

Duchenne muscular dystrophy, like many other diseases of genetic origin, present challenging therapeutic scenarios. The CRISPR-Cas system represents an approach for correction of diverse genetic defects. The CRISPR (clustered regularly interspaced short palindromic repeats) system functions as an adaptive immune system in bacteria and archaea that defends against phage infection. In this system, an endonuclease is guided to specific genomic sequences by a single guide RNA (sgRNA), resulting in DNA cutting near a protospacer adjacent motif (PAM) sequence. Previously, CRISPR-Cas9 was used to correct the DMD mutation in mice and human cells. However, many challenges remain to be addressed. For example, Streptococcus pyogenes Cas9 (SpCas9), currently the most widely used Cas9 endonuclease, has a G-rich PAM requirement (NGG) that excludes genome editing of AT-rich regions. Additionally, the large size of SpCas9 reduces the efficiency of packaging and delivery in low-capacity viral vectors, such as Adeno-associated virus (AAV) vectors. The Cas9 endonuclease from Staphylococcus aureus (SaCas9), although smaller in size than SpCas9, has a PAM sequence (NNGRRT) that is longer and more complex, thus limiting the range of its genomic targets (Ran et al., 2015). Smaller CRISPR enzymes with greater flexibility in recognition sequence and comparable cutting efficiency would facilitate precision gene editing, especially for translational applications.

As demonstrated by the disclosure, an RNA-guided endonuclease, named Cpf1 (CRISPR from Prevotella and Francisella 1), is effective for mammalian genome cleavage.

Cpf1 has several unique features that expand its genome editing potential when compared to Cas9: Cpf1-mediated cleavage is guided by a single and short crRNA (abbreviated as gRNA), whereas Cas9-mediated cleavage is guided by a hybrid of CRISPR RNA (crRNA) and a long trans-activating crRNA (tracrRNA). Cpf1 prefers a T-rich PAM at the 5′-end of a protospacer, while Cas9 requires a G-rich PAM at the 3′ end of the target sequence. Cpf1-mediated cleavage produces a sticky end distal to the PAM site, which activates DNA repair machinery, while Cas9 cutting generates a blunt end. Cpf1 also has RNase activity, which can process precursor crRNAs to mature crRNAs. Like Cas9, Cpf1 binds to a targeted genomic site and generates a double-stranded break (DSB), which is then repaired either by non-homologous end-joining (NHEJ) or by homology-directed repair (HDR) if an exogenous template is provided. Prior to the instant disclosure, neither had the advantages of Cpf1 over Cas9 been appreciated nor had the use of Cpf1 for correction of genetic mutations in mammalian cells and animal models of disease been demonstrated. Here, the inventors show that Cpf1 provides a robust and efficient RNA-guided genome editing system that permanently corrects DMD mutations by different strategies, thereby restoring dystrophin expression and preventing progression of the disease. These findings provide a new approach for the permanent correction of human genetic mutations. These and other aspects of the disclosure are reproduced below.

### I. DUCHENNE MUSCULAR DYSTROPHY

A. Background

Duchenne muscular dystrophy (DMD) is a recessive X-linked form of muscular dystrophy, affecting around 1 in 3,500 boys, which results in muscle degeneration and premature death. The disorder is caused by a mutation in the gene dystrophin (See GenBank Accession No. NC_000023.11), located on the human X chromosome, which codes for the protein dystrophin (GenBank Accession No. AAA53189; SEQ ID NO. 383), the sequence of which is reproduced below:

| | |
|---|---|
| 1 | mlwweevedc yeredvqkkt ftkwvnaqfs kfgkqhienl fsdlqdgrrl ldllegltgq |
| 61 | klpkekgstr vhalnnvnka lrvlqnnnvd lvnigstdiv dgnhkltlgl iwniilhwqv |
| 121 | knvmknimag lqqtnsekil lswvrqstrn ypqvnvinft tswsdglaln alihshrpdl |
| 181 | fdwnsvvcqq satqrlehaf niaryqlgie klldpedvdt typdkksilm yitslfqvlp |
| 241 | qqvsieaiqe vemlprppkv tkeehfqlhh qmhysqqitv slaqgyerts spkprfksya |
| 301 | ytqaayvtts dptrspfpsq hleapedksf gsslmesevn ldryqtalee vlswllsaed |
| 361 | tlqaqgeisn dvevvkdqfh thegymmdlt ahqgrvgnil qlgskligtg klsedeetev |
| 421 | qeqmnllnsr weclrvasme kqsnlhrvlm dlqnqklkel ndwltkteer trkmeeeplg |
| 481 | pdledlkrqv qqhkvlqedl eqeqvrvnsl thmvvvvdes sgdhataale eqlkvlgdrw |
| 541 | anicrwtedr wvllqdillk wqrlteeqcl fsawlseked avnkihttgf kdqnemlssl |
| 601 | qklavlkadl ekkkqsmgkl yslkqdllst lknksvtqkt eawldnfarc wdnlvqklek |
| 661 | staqisqavt ttqpsltqtt vmetvttvtt reqilvkhaq eelpppppqk krqitvdsei |
| 721 | rkrldvdite lhswitrsea vlqspefaif rkegnfsdlk ekvnaierek aekfrklqda |
| 781 | srsaqalveq mvnegvnads ikqaseqlns rwiefcqlls erlnwleyqn niiafynqlq |
| 841 | qleqmtttae nwlkiqpttp septaiksql kickdevnrl sglqpqierl kiqsialkek |
| 901 | gqgpmfldad fvaftnhfkq vfsdvqarek elqtifdtlp pmryqetmsa irtwvqqset |
| 961 | klsipqlsvt dyeimeqrlg elqalqsslq eqqsglyyls ttvkemskka pseisrkyqs |
| 1021 | efeeiegrwk klssqlvehc qkleeqmnkl rkiqnhiqtl kkwmaevdvf lkeewpalgd |
| 1081 | seilkkqlkq crllvsdiqt iqpslnsvne ggqkikneae pefasrlete lkelntqwdh |
| 1141 | mcqqvyarke alkgglektv slqkdlsemh ewmtqaeeey lerdfeyktp delqkaveem |
| 1201 | krakeeaqqk eakvklltes vnsviaqapp vaqealkkel etlttnyqwl ctrlngkckt |
| 1261 | leevwacwhe llsylekank wlnevefklk ttenipggae eisevldsle nlmrhsednp |
| 1321 | nqirilaqtl tdggvmdeli neeletfnsr wrelheeavr rqklleqsiq saqetekslh |
| 1381 | liqesltfid kqlaayiadk vdaaqmpqea qkiqsdltsh eisleemkkh nqgkeaaqrv |
| 1441 | lsqidvaqkk lqdvsmkfrl fqkpanfelr lqeskmilde vkmhlpalet ksveqevvqs |
| 1501 | qlnhcvnlyk slsevkseve mviktgrqiv qkkqtenpke ldervtalkl hynelgakvt |
| 1561 | erkqqlekcl klsrkmrkem nvltewlaat dmeltkrsav egmpsnldse vawgkatqke |
| 1621 | iekqkvhlks itevgealkt vlgkketlve dklsllnsnw iavtsraeew lnllleyqkh |
| 1681 | metfdqnvdh itkwiiqadt lldesekkkp qqkedvlkrl kaelndirpk vdstrdqaan |
| 1741 | lmanrgdhcr klvepqisel nhrfaaishr iktgkasipl keleqfnsdi qkllepleae |
| 1801 | iqqgvnlkee dfnkdmnedn egtvkellqr gdnlqqritd erkreeikik qqllqtkhna |
| 1861 | lkdlrsqrrk kaleishqwy qykrqaddll kclddiekkl aslpeprder kikeidrelq |
| 1921 | kkkeelnavr rqaeglsedg aamaveptqi qlskrwreie skfaqfrrln faqihtvree |
| 1981 | tmmvmtedmp leisyvpsty lteithvsqa lleveqllna pdlcakdfed lfkqeeslkn |
| 2041 | ikdslqqssg ridiihskkt aalqsatpve rvklqealsq ldfqwekvnk mykdrqgrfd |
| 2101 | rsvekwrrfh ydikifnqwl teaeqflrkt qipenwehak ykwylkelqd gigqrqtvvr |
| 2161 | tlnatgeeii qqssktdasi lqeklgslnl rwqevckqls drkkrleeqk nilsefqrdl |
| 2221 | nefvlwleea dniasiplep gkeqqlkekl eqvkllveel plrqgilkql netggpvlvs |
| 2281 | apispeeqdk lenklkqtnl qwikvsralp ekqgeieaqi kdlgqlekkl edleeqlnhl |
| 2341 | llwlspirnq leiynqpnqe gpfdvqetei avqakqpdve eilskgqhly kekpatqpvk |
| 2401 | rkledlssew kavnrllqel rakqpdlapg lttigasptq tvtlvtqpvv tketaiskle |
| 2461 | mpsslmlevp aladfnrawt eltdwlslld qviksqrvmv gdledinemi ikqkatmqdl |
| 2521 | eqrrpqleel itaaqnlknk tsnqeartii tdrieriqnq wdevqehlqn rrqqlnemlk |
| 2581 | dstqwleake eaeqvlgqar akleswkegp ytvdaiqkki tetkqlakdl rqwqtnvdva |
| 2641 | ndlalkllrd ysaddtrkvh miteninasw rsihkrvser eaaleethrl lqqfpldlek |
| 2701 | flawlteaet tanvlqdatr kerlledskg vkelmkqwqd lqgeieahtd vyhnldensq |
| 2761 | kilrslegsd davllqrrld nmnfkwselr kkslnirshl eassdqwkrl hlslqellvw |
| 2821 | lqlkddelsr qapiggdfpa vqkqndvhra fkrelktkep vimstletvr iflteqpleg |
| 2881 | leklyqepre lppeeraqnv trllrkqaee vnteweklnl hsadwqrkid etlerlqelq |
| 2941 | eatdeldlkl rqaevikgsw qpvgdllids lqdhlekvka lrgeiaplke nvshvndlar |
| 3001 | qlttlgiqls pynlstledl ntrwkllqva vedrvrqlhe ahrdfgpasq hflstsvqgp |
| 3061 | weraispnkv pyyinhetqt tcwdhpkmte lyqsladlnn vrfsayrtam klrrlqkalc |
| 3121 | ldllslsaac daldqhnlkq ndqpmdilqi inclttiydr leqehnnlvn vplcvdmcln |
| 3181 | wllnvydtgr tgrirvlsfk tgiislckah ledkyrylfk qvasstgfcd qrrlglllhd |
| 3241 | siqiprqlge vasfggsnie psvrscfqfa nnkpeieaal fldwmrlepq smvwlpvlhr |
| 3301 | vaaaetakhq akcnickecp iigfryrslk hfnydicqsc ffsgrvakgh kmhypmveyc |
| 3361 | tpttsgedvr dfakvlknkf rtkryfakhp rmgylpvqtv legdnmetpv tlinfwpvds |
| 3421 | apasspqlsh ddthsriehy asrlaemens ngsylndsis pnesiddehl liqhycqsln |
| 3481 | qdsplsqprs paqilisles eergeleril adleeenrnl qaeydrlkqq hehkglsplp |
| 3541 | sppemmptsp qsprdaelia eakllrqhkg rlearmqile dhnkqlesql hrlrqlleqp |
| 3601 | qaeakvngtt vsspstslqr sdssqpmllr vvgsqtsdsm geedllsppq dtstgleevm |
| 3661 | eqlnnsfpss rgrntpgkpm redtm |

In humans, dystrophin mRNA contains 79 exons. Dystrophin mRNA is known to be alternatively spliced, resulting in various isoforms. Exemplary dystrophin isoforms are listed in Table 1.

In humans, dystrophin mRNA contains 79 exons. Dystrophin mRNA is known to be alternatively spliced, resulting in various isoforms of the protein. Exemplary dystrophin isoforms are listed in Table 1.

TABLE 1
Dystrophin isoforms

| | | Nucleic | | | |
|---|---|---|---|---|---|
| | | Acid | | Protein | |
| Sequence | Nucleic Acid | SEQ ID | Protein Accession | SEQ ID | |
| Name* | Accession No.* | NO: | No.* | NO: | Description |
| DMD Genomic | NC_000023.11 | None | None | None | Sequence from Human |
| Sequence | (positions | | | | X Chromosome (at |
| | 31119219 to | | | | positions Xp21.2 to |
| | 33339609) | | | | p21.1) from Assembly |
| | | | | | GRCh38.p7 |
| | | | | | (GCF_000001405.33) |
| Dystrophin | NM_000109.3 | 384 | NP_000100.2 | 385 | Transcript Variant: |
| Dp427c isoform | | | | | transcript Dp427c is |
| | | | | | expressed |
| | | | | | predominantly in |
| | | | | | neurons of the cortex |
| | | | | | and the CA regions of |
| | | | | | the hippocampus. It |
| | | | | | uses a unique |
| | | | | | promoter/exon 1 |
| | | | | | located about 130 kb |
| | | | | | upstream of the |
| | | | | | Dp427m transcript |
| | | | | | promoter. The |
| | | | | | transcript includes the |
| | | | | | common exon 2 of |
| | | | | | transcript Dp427m and |
| | | | | | has a similar length of |
| | | | | | 14 kb. The Dp427c |
| | | | | | isoform contains a |
| | | | | | unique N-terminal |
| | | | | | MED sequence, instead |
| | | | | | of the |
| | | | | | MLWWEEVEDCY |
| | | | | | (SEQ ID NO: 3) |
| | | | | | sequence of isoform |
| | | | | | Dp427m. The |
| | | | | | remainder of isoform |
| | | | | | Dp427c is identical to |
| | | | | | isoform Dp427m. |
| Dystrophin | NM_004006.2 | 386 | NP_003997.1 | 387 | Transcript Variant: |
| Dp427m | | | | | transcript Dp427m |
| isoform | | | | | encodes the main |
| | | | | | dystrophin protein |
| | | | | | found in muscle. As a |
| | | | | | result of alternative |
| | | | | | promoter use, exon 1 |
| | | | | | encodes a unique N- |
| | | | | | terminal |
| | | | | | MLWWEEVEDCY |
| | | | | | (SEQ ID NO: 3) aa |
| | | | | | sequence. |
| Dystrophin | NM_004009.3 | 388 | NP_004000.1 | 389 | Transcript Variant: |
| Dp427p1 | | | | | transcript Dp427p1 |
| isoform | | | | | initiates from a unique |
| | | | | | promoter/exon 1 |
| | | | | | located in what |
| | | | | | corresponds to the first |
| | | | | | intron of transcript |
| | | | | | Dp427m. The transcript |
| | | | | | adds the common exon |
| | | | | | 2 of Dp427m and has a |
| | | | | | similar length (14 kb). |
| | | | | | The Dp427p1 isoform |
| | | | | | replaces the |
| | | | | | MLWWEEVEDCY |
| | | | | | (SEQ ID NO: 3)-start |
| | | | | | of Dp427m with a |
| | | | | | unique N-terminal |
| | | | | | MSEVSSD (SEQ ID |
| | | | | | NO: 8) aa sequence. |
| Dystrophin | NM_004011.3 | 390 | NP_004002.2 | 391 | Transcript Variant: |
| Dp260-1 | | | | | transcript Dp260-1 uses |
| isoform | | | | | exons 30-79, and |
| | | | | | originates from a |
| | | | | | promoter/exon 1 |
| | | | | | sequence located in |
| | | | | | intron 29 of the |
| | | | | | dystrophin gene. As a |
| | | | | | result, Dp260-1 |
| | | | | | contains a 95 bp exon 1 |
| | | | | | encoding a unique N- |
| | | | | | terminal 16 aa |
| | | | | | MTEIILLIFFPAYFLN- |
| | | | | | sequence that replaces |
| | | | | | amino acids 1-1357 of |
| | | | | | the full-length |
| | | | | | dystrophin product |
| | | | | | (Dp427m isoform). |
| Dystrophin | NM_004012.3 | 392 | NP_004003.1 | 393 | Transcript Variant: |
| Dp260-2 | | | | | transcript Dp260-2 uses |
| isoform | | | | | exons 30-79, starting |
| | | | | | from a promoter/exon 1 |
| | | | | | sequence located in |
| | | | | | intron 29 of the |
| | | | | | dystrophin gene that is |
| | | | | | alternatively spliced |
| | | | | | and lacks N-terminal |
| | | | | | amino acids 1-1357 of |
| | | | | | the full length |
| | | | | | dystrophin (Dp427m |
| | | | | | isoform). The Dp260-2 |
| | | | | | transcript encodes a |
| | | | | | unique N-terminal |
| | | | | | MSARKLRNLSYKK |
| | | | | | sequence. |
| Dystrophin | NM_004013.2 | 394 | NP_004004.1 | 395 | Transcript Variant: |
| Dp140 isoform | | | | | Dp140 transcripts use |
| | | | | | exons 45-79, starting at |
| | | | | | a promoter/exon 1 |
| | | | | | located in intron 44. |
| | | | | | Dp140 transcripts have |
| | | | | | a long (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140) contains all of |
| | | | | | the exons. |
| Dystrophin | NM_004014.2 | 396 | NP_004005.1 | 397 | Transcript Variant: |
| Dp116 isoform | | | | | transcript Dp116 uses |
| | | | | | exons 56-79, starting |
| | | | | | from a promoter/exon 1 |
| | | | | | within intron 55. As a |
| | | | | | result, the Dp116 |
| | | | | | isoform contains a |
| | | | | | unique N-terminal |
| | | | | | MLHRKTYHVK aa |
| | | | | | sequence, instead of aa |
| | | | | | 1-2739 of dystrophin. |
| | | | | | Differential splicing |
| | | | | | produces several |
| | | | | | Dp116-subtypes. The |
| | | | | | Dp116 isoform is also |
| | | | | | known as S-dystrophin |
| | | | | | or apo-dystrophin-2. |
| Dystrophin | NM_004015.2 | 398 | NP_004006.1 | 399 | Transcript Variant: |
| Dp71 isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71) includes both |
| | | | | | exons 71 and 78. |
| Dystrophin | NM_004016.2 | 400 | NP_004007.1 | 401 | Transcript Variant: |
| Dp71b isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71b) lacks exon 78 |
| | | | | | and encodes a protein |
| | | | | | with a different C- |
| | | | | | terminus than Dp71 and |
| | | | | | Dp71a isoforms. |
| Dystrophin | NM_004017.2 | 402 | NP_004008.1 | 403 | Transcript Variant: |
| Dp71a isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71a) lacks exon 71. |
| Dystrophin | NM_004018.2 | 404 | NP_004009.1 | 405 | Transcript Variant: |
| Dp71ab isoform | | | | | Dp71 transcripts use |
| | | | | | exons 63-79 with a |
| | | | | | novel 80- to 100-nt |
| | | | | | exon containing an |
| | | | | | ATG start site for a |
| | | | | | new coding sequence of |
| | | | | | 17 nt. The short coding |
| | | | | | sequence is in-frame |
| | | | | | with the consecutive |
| | | | | | dystrophin sequence |
| | | | | | from exon 63. |
| | | | | | Differential splicing of |
| | | | | | exons 71 and 78 |
| | | | | | produces at least four |
| | | | | | Dp71 isoforms. Of |
| | | | | | these, this transcript |
| | | | | | (Dp71ab) lacks both |
| | | | | | exons 71 and 78 and |
| | | | | | encodes a protein with |
| | | | | | a C-terminus like |
| | | | | | isoform Dp71b. |
| Dystrophin | NM_004019.2 | 406 | NP_004010.1 | 407 | Transcript Variant: |
| Dp40 isoform | | | | | transcript Dp40 uses |
| | | | | | exons 63-70. The 5′ |
| | | | | | UTR and encoded first |
| | | | | | 7 aa are identical to that |
| | | | | | in transcript Dp71, but |
| | | | | | the stop codon lies at |
| | | | | | the splice junction of |
| | | | | | the exon/intron 70. The |
| | | | | | 3′ UTR includes nt |
| | | | | | from intron 70 which |
| | | | | | includes an alternative |
| | | | | | polyadenylation site. |
| | | | | | The Dp40 isoform |
| | | | | | lacks the normal C- |
| | | | | | terminal end of full- |
| | | | | | length dystrophin (aa |
| | | | | | 3409-3685). |
| Dystrophin | NM_004020.3 | 408 | NP_004011.2 | 409 | Transcript Variant: |
| Dp140c isoform | | | | | Dp140 transcripts use |
| | | | | | exons 45-79, starting at |
| | | | | | a promoter/exon 1 |
| | | | | | located in intron 44. |
| | | | | | Dp140 transcripts have |
| | | | | | a long (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140c) lacks exons |
| | | | | | 71-74. |
| Dystrophin | NM_004021.2 | 410 | NP_004012.1 | 411 | Transcript Variant: |
| Dp140b | | | | | Dp140 transcripts use |
| isoform | | | | | exons 45-79, starting at |
| | | | | | a promoter/exon 1 |
| | | | | | located in intron 44. |
| | | | | | Dp140 transcripts have |
| | | | | | a long (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140b) lacks exon 78 |
| | | | | | and encodes a protein |
| | | | | | with a unique C- |
| | | | | | terminus. |
| Dystrophin | NM_004022.2 | 412 | NP_004013.1 | 413 | Transcript Variant: |
| Dp140ab | | | | | Dp140 transcripts use |
| isoform | | | | | exons 45-79, starting at |
| | | | | | a promoter/exon 1 |
| | | | | | located in intron 44. |
| | | | | | Dp140 transcripts have |
| | | | | | a long (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140ab) lacks exons |
| | | | | | 71 and 78 and encodes |
| | | | | | a protein with a unique |
| | | | | | C-terminus. |
| Dystrophin | NM_004023.2 | 414 | NP_004014.1 | 415 | Transcript Variant: |
| Dp140bc | | | | | Dp140 transcripts use |
| isoform | | | | | exons 45-79, starting at |
| | | | | | a promoter/exon 1 |
| | | | | | located in intron 44. |
| | | | | | Dp140 transcripts have |
| | | | | | a long (1 kb) 5′ UTR |
| | | | | | since translation is |
| | | | | | initiated in exon 51 |
| | | | | | (corresponding to aa |
| | | | | | 2461 of dystrophin). In |
| | | | | | addition to the |
| | | | | | alternative promoter |
| | | | | | and exon 1, differential |
| | | | | | splicing of exons 71-74 |
| | | | | | and 78 produces at least |
| | | | | | five Dp140 isoforms. |
| | | | | | Of these, this transcript |
| | | | | | (Dp140bc) lacks exons |
| | | | | | 71-74 and 78 and |
| | | | | | encodes a protein with |
| | | | | | a unique C-terminus. |
| Dystrophin | XM_006724469.3 | 416 | XP_006724532.1 | 417 | |
| isoform X2 | | | | | |
| Dystrophin | XM_011545467.1 | 418 | XP_011543769.1 | 419 | |
| isoform X5 | | | | | |
| Dystrophin | XM_006724473.2 | 420 | XP_006724536.1 | 421 | |
| isoform X6 | | | | | |
| Dystrophin | XM_006724475.2 | 422 | XP_006724538.1 | 423 | |
| isoform X8 | | | | | |
| Dystrophin | XM_017029328.1 | 424 | XP_016884817.1 | 425 | |
| isoform X4 | | | | | |
| Dystrophin | XM_006724468.2 | 426 | XP_006724531.1 | 427 | |
| isoform X1 | | | | | |
| Dystrophin | XM_017029331.1 | 428 | XP_016884820.1 | 429 | |
| isoform X13 | | | | | |
| Dystrophin | XM_006724470.3 | 430 | XP_006724533.1 | 431 | |
| isoform X3 | | | | | |
| Dystrophin | XM_006724474.3 | 432 | XP_006724537.1 | 433 | |
| isoform X7 | | | | | |
| Dystrophin | XM_011545468.2 | 434 | XP_011543770.1 | 435 | |
| isoform X9 | | | | | |
| Dystrophin | XM_017029330.1 | 436 | XP_016884819.1 | 437 | |
| isoform X11 | | | | | |
| Dystrophin | XM_017029329.1 | 438 | XP_016884818.1 | 439 | |
| isoform X10 | | | | | |
| Dystrophin | XM_011545469.1 | 440 | XP_011543771.1 | 441 | |
| isoform X12 | | | | | |

Dystrophin is an important component within muscle tissue that provides structural stability to the dystroglycan complex (DGC) of the cell membrane. While both sexes can carry the mutation, females are rarely affected with the skeletal muscle form of the disease.

B. Symptoms

Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that lead to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of Duchenne muscular dystrophy, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
Awkward manner of walking, stepping, or running—(patients tend to walk on their forefeet, because of an increased calf muscle tone. Also, toe walking is a compensatory adaptation to knee extensor weakness.)
Frequent falls
Fatigue
Difficulty with motor skills (running, hopping, jumping)
Lumbar hyperlordosis, possibly leading to shortening of the hip-flexor muscles. This has an effect on overall posture and a manner of walking, stepping, or running.
Muscle contractures of Achilles tendon and hamstrings impair functionality because the muscle fibers shorten and fibrose in connective tissue
Progressive difficulty walking
Muscle fiber deformities
Pseudohypertrophy (enlarging) of tongue and calf muscles. The muscle tissue is eventually replaced by fat and connective tissue, hence the term pseudohypertrophy.
Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain
Eventual loss of ability to walk (usually by the age of 12)
Skeletal deformities (including scoliosis in some cases)
Trouble getting up from lying or sitting position

The condition can often be observed clinically from the moment the patient takes his first steps, and the ability to walk usually completely disintegrates between the time the boy is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremities muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.
Abnormal heart muscle (cardiomyopathy)
Congestive heart failure or irregular heart rhythm (arrhythmia)
Deformities of the chest and back (scoliosis)
Enlarged muscles of the calves, buttocks, and shoulders (around age 4 or 5). These muscles are eventually replaced by fat and connective tissue (pseudohypertrophy).
Loss of muscle mass (atrophy)
Muscle contractures in the heels, legs
Muscle deformities
Respiratory disorders, including pneumonia and swallowing with food or fluid passing into the lungs (in late stages of the disease)

C. Causes

Duchenne muscular dystrophy (DMD) is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Exon deletions preceding exon 51 of the human DMD gene, which disrupt the open reading frame (ORF) by juxtaposing out of frame exons, represent the most common type of human DMD mutation. Skipping of exon 51 can, in principle, restore the DMD ORF in 13% of DMD patients with exon deletions.

Duchenne muscular dystrophy has an incidence of 1 in 3,500 male infants. Mutations within the dystrophin gene can either be inherited or occur spontaneously during germline transmission.

D. Diagnosis

Genetic counseling is advised for people with a family history of the disorder. Duchenne muscular dystrophy can be detected with about 95% accuracy by genetic studies performed during pregnancy.

DNA Test.

The muscle-specific isoform of the dystrophin gene is composed of 79 exons, and DNA testing and analysis can usually identify the specific type of mutation of the exon or exons that are affected. DNA testing confirms the diagnosis in most cases.

Muscle Biopsy.

If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition.

Over the past several years DNA tests have been developed that detect more of the many mutations that cause the condition, and muscle biopsy is not required as often to confirm the presence of Duchenne's.

Prenatal Tests.

DMD is carried by an X-linked recessive gene. Males have only one X chromosome, so one copy of the mutated gene will cause DMD. Fathers cannot pass X-linked traits on to their sons, so the mutation is transmitted by the mother.

If the mother is a carrier, and therefore one of her two X chromosomes has a DMD mutation, there is a 50% chance that a female child will inherit that mutation as one of her two X chromosomes, and be a carrier. There is a 50% chance that a male child will inherit that mutation as his one X chromosome, and therefore have DMD.

Prenatal tests can tell whether their unborn child has the most common mutations.

There are many mutations responsible for DMD, and some have not been identified, so genetic testing only works when family members with DMD have a mutation that has been identified.

Prior to invasive testing, determination of the fetal sex is important; while males are sometimes affected by this X-linked disease, female DMD is extremely rare. This can be achieved by ultrasound scan at 16 weeks or more recently by free fetal DNA testing. Chorion villus sampling (CVS) can be done at 11-14 weeks, and has a 1% risk of miscarriage. Amniocentesis can be done after 15 weeks, and has a 0.5% risk of miscarriage. Fetal blood sampling can be done at about 18 weeks. Another option in the case of unclear genetic test results is fetal muscle biopsy.

E. Treatment

There is no current cure for DMD, and an ongoing medical need has been recognized by regulatory authorities. Phase 1-2a trials with exon skipping treatment for certain mutations have halted decline and produced small clinical improvements in walking. Treatment is generally aimed at controlling the onset of symptoms to maximize the quality of life, and include the following:
Corticosteroids such as prednisolone and deflazacort increase energy and strength and defer severity of some symptoms.
Randomized control trials have shown that beta-2-agonists increase muscle strength but do not modify disease progression. Follow-up time for most RCTs on beta2-agonists is only around 12 months and hence results cannot be extrapolated beyond that time frame.
Mild, non-jarring physical activity such as swimming is encouraged. Inactivity (such as bed rest) can worsen the muscle disease.
Physical therapy is helpful to maintain muscle strength, flexibility, and function.
Orthopedic appliances (such as braces and wheelchairs) may improve mobility and the ability for self-care. Form-fitting removable leg braces that hold the ankle in place during sleep can defer the onset of contractures.
Appropriate respiratory support as the disease progresses is important.
   Comprehensive multi-disciplinary care standards/guidelines for DMD have been developed by the Centers for Disease Control and Prevention (CDC), and are available at www.treat-nmd.eu/dmd/care/diagnosis-management-DMD.

DMD generally progresses through five stages, as outlined in Bushby et al., Lancet Neurol., 9(1): 77-93 (2010) and Bushby et al., Lancet Neurol., 9(2): 177-198 (2010), incorporated by reference in their entireties. During the presymptomatic stage, patients typically show developmental delay, but no gait disturbance. During the early ambulatory stage, patients typically show the Gowers' sign, waddling gait, and toe walking. During the late ambulatory stage, patients typically exhibit an increasingly labored gait and begin to lose the ability to climb stairs and rise from the floor. During the early non-ambulatory stage, patients are typically able to self-propel for some time, are able to maintain posture, and may develop scoliosis. During the late non-ambulatory stage, upper limb function and postural maintenance is increasingly limited.

In some embodiments, treatment is initiated in the presymptomatic stage of the disease. In some embodiments, treatment is initiated in the early ambulatory stage. In some embodiments, treatment is initiated in the late ambulatory stage. In embodiments, treatment is initiated during the early non-ambulatory stage. In embodiments, treatment is initiated during the late non-ambulatory stage.

1. Physical Therapy

Physical therapists are concerned with enabling patients to reach their maximum physical potential. Their aim is to:
minimize the development of contractures and deformity by developing a program of stretches and exercises where appropriate
anticipate and minimize other secondary complications of a physical nature by recommending bracing and durable medical equipment
monitor respiratory function and advise on techniques to assist with breathing exercises and methods of clearing secretions

2. Respiration Assistance

Modern “volume ventilators/respirators,” which deliver an adjustable volume (amount) of air to the person with each breath, are valuable in the treatment of people with muscular dystrophy related respiratory problems. The ventilator may require an invasive endotracheal or tracheotomy tube through which air is directly delivered, but, for some people non-invasive delivery through a face mask or mouthpiece is sufficient. Positive airway pressure machines, particularly bi-level ones, are sometimes used in this latter way. The respiratory equipment may easily fit on a ventilator tray on the bottom or back of a power wheelchair with an external battery for portability.

Ventilator treatment may start in the mid to late teens when the respiratory muscles can begin to collapse. If the vital capacity has dropped below 40% of normal, a volume ventilator/respirator may be used during sleeping hours, a time when the person is most likely to be under ventilating (“hypoventilating”). Hypoventilation during sleep is determined by a thorough history of sleep disorder with an oximetry study and a capillary blood gas (See Pulmonary Function Testing). A cough assist device can help with excess mucus in lungs by hyperinflation of the lungs with positive air pressure, then negative pressure to get the mucus up. If the vital capacity continues to decline to less than 30 percent of normal, a volume ventilator/respirator may also be needed during the day for more assistance. The person gradually will increase the amount of time using the ventilator/respirator during the day as needed.

F. Prognosis

Duchenne muscular dystrophy is a progressive disease which eventually affects all voluntary muscles and involves the heart and breathing muscles in later stages. The life expectancy is currently estimated to be around 25, but this varies from patient to patient. Recent advancements in medicine are extending the lives of those afflicted. The Muscular Dystrophy Campaign, which is a leading UK charity focusing on all muscle disease, states that “with high standards of medical care young men with Duchenne muscular dystrophy are often living well into their 30s.”

In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

Curiously, in the mdx mouse model of Duchenne muscular dystrophy, the lack of dystrophin is associated with increased calcium levels and skeletal muscle myonecrosis. The intrinsic laryngeal muscles (ILM) are protected and do not undergo myonecrosis. ILM have a calcium regulation system profile suggestive of a better ability to handle calcium changes in comparison to other muscles, and this may provide a mechanistic insight for their unique pathophysiological properties. The ILM may facilitate the development of novel strategies for the prevention and treatment of muscle wasting in a variety of clinical scenarios.

### II. CRISPR SYSTEMS

A. CRISPRs

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

B. Cas Nucleases

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (˜30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. The team demonstrated that they could disable one or both sites while preserving Cas9's ability to locate its target DNA. combined tracrRNA and spacer RNA into a “single-guide RNA” molecule that, mixed with Cas9, could find and cut the correct DNA targets proposed that such synthetic guide RNAs might be able to be used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Wang et al. showed that coinjection of Cas9 mRNA and sgRNAs into the germline (zygotes) generated nice with mutations. Delivery of Cas9 DNA sequences also is contemplated.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein. See also U.S. Patent Publication 2014/0068797, which is incorporated by reference in its entirety.

C. Cpf1 Nucleases

Clustered Regularly Interspaced Short Palindromic Repeats from Prevotella and Francisella 1 or CRISPR/Cpf1 is a DNA-editing technology which shares some similarities with the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Cpf1 appears in many bacterial species. The ultimate Cpf1 endonuclease that was developed into a tool for genome editing was taken from one of the first 16 species known to harbor it.

In embodiments, the Cpf1 is a Cpf1 enzyme from Acidaminococcus (species BV3L6, UniProt Accession No. U2UMQ6; SEQ ID NO. 442), having the sequence set forth below:

| | |
|---|---|
| 1 | mtqfegftnl yqvsktlrfe lipqgktlkh iqeqgfieed karndhykel kpiidriykt |
| 61 | yadqclqlvq ldwenlsaai dsyrkektee trnalieeqa tyrnaihdyf igrtdnltda |
| 121 | inkrhaeiyk glfkaelfng kvlkqlgtvt ttehenallr sfdkfttyfs gfyenrknvf |
| 181 | saedistaip hrivqdnfpk fkenchiftr litavpslre hfenvkkaig ifvstsieev |
| 241 | fsfpfynqll tqtqidlynq llggisreag tekikglnev lnlaiqknde tahiiaslph |
| 301 | rfiplfkqil sdrntlsfil eefksdeevi qsfckyktll rnenvletae alfnelnsid |
| 361 | lthifishkk letissalcd hwdtlrnaly erriseltgk itksakekvq rslkhedinl |
| 421 | qeiisaagke lseafkqkts eilshahaal dqplpttlkk qeekeilksq ldsllglyhl |
| 481 | ldwfavdesn evdpefsarl tgiklemeps lsfynkarny atkkpysvek fklnfqmptl |
| 541 | asgwdvnkek nngailfvkn glyylgimpk qkgrykalsf eptektsegf dkmyydyfpd |
| 601 | aakmipkcst qlkavtahfq thttpillsn nfiepleitk eiydlnnpek epkkfqtaya |
| 661 | kktgdqkgyr ealckwidft rdflskytkt tsidlsslrp ssqykdlgey yaelnpllyh |
| 721 | isfqriaeke imdavetgkl ylfqiynkdf akghhgkpnl htlywtglfs penlaktsik |
| 781 | lngqaelfyr pksrmkrmah rlgekmlnkk lkdqktpipd tlyqelydyv nhrlshdlsd |
| 841 | earallpnvi tkevsheiik drrftsdkff fhvpitlnyq aanspskfnq rvnaylkehp |
| 901 | etpiigidrg ernliyitvi dstgkileqr slntiqqfdy qkkldnreke rvaarqawsv |
| 961 | vgtikdlkqg ylsqviheiv dlm1iyqavv vlenlnfgfk skrtgiaeka vyqqfekmli |
| 1021 | dklnclvlkd ypaekvggvl npyqltdqft sfakmgtqsg flfyvpapyt skidpltgfv |
| 1081 | dpfvwktikn hesrkhfleg fdflhydvkt gdfilhfkmn rnlsfqrglp gfmpawdivf |
| 1141 | eknetqfdak gtpfiagkri vpvienhrft gryrdlypan elialleekg ivfrdgsnil |
| 1201 | pkllenddsh aidtmvalir svlqmrnsna atgedyinsp vrdlngvcfd srfqnpewpm |
| 1261 | dadangayhi alkgqlllnh lkeskdlklq ngisnqdwla yiqelrn |

In some embodiments, the Cpf1 is a Cpf1 enzyme from Lachnospiraceae (species ND2006, UniProt Accession No. AOA182DWE3; SEQ ID NO. 443), having the sequence set forth below:

| | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSFNGFTT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK |

In some embodiments, the Cpf1 is codon optimized for expression in mammalian cells. In some embodiments, the Cpf1 is codon optimized for expression in human cells.

In some embodiments, the compositions of the disclosure include a small version of a Cas9 from the bacterium Staphylococcus aureus. The small version of the Cas9 provides advantages over wild type or full length Cas9.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 does not require a tracrRNA. Therefore, functional Cpf1 gRNAs of the disclosure may comprise or consist of a crRNA. This benefits genome editing because Cpf1 is not only a smaller nuclease than Cas9, but also it has a smaller sgRNA molecule (proximately half as many nucleotides as Cas9).

The Cpf1-gRNA (e.g. Cpf1-crRNA) complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system comprises or consists of a Cpf1 enzyme and a guide RNA that finds and positions the complex at the correct spot on the double helix to cleave target DNA. In its native bacterial hosts, CRISPR/Cpf1 systems activity has three stages:
Adaptation, during which Cas1 and Cas2 proteins facilitate the adaptation of small fragments of DNA into the CRISPR array;
Formation of crRNAs: processing of pre-cr-RNAs producing of mature crRNAs to guide the Cas protein; and
Interference, in which the Cpf1 is bound to a crRNA to form a binary complex to identify and cleave a target DNA sequence.

This system has been modified to utilize non-naturally occurring crRNAs, which guide Cpf1 to a desired target sequence in a non-bacterial cell, such as a mammalian cell.

D. gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type III promoter U6. Synthetic gRNAs are slightly over 100 bp at the minimum length and contain a portion which is targets the 20 protospacer nucleotides immediately preceding the PAM sequence NGG; gRNAs do not contain a PAM sequence.

In some embodiments, the gRNA targets a site within a wildtype dystrophin gene. In some embodiments, the gRNA targets a site within a mutant dystrophin gene. In some embodiments, the gRNA targets a dystrophin intron. In some embodiments, the gRNA targets dystrophin exon. In some embodiments, the gRNA targets a site in a dystrophin exon that is expressed and is present in one or more of the dystrophin isoforms shown in Table 1. In embodiments, the gRNA targets a dystrophin splice site. In some embodiments, the gRNA targets a splice donor site on the dystrophin gene. In embodiments, the gRNA targets a splice acceptor site on the dystrophin gene.

In embodiments, the guide RNA targets a mutant DMD exon. In some embodiments, the mutant exon is exon 23 or 51. In some embodiments, the guide RNA targets at least one of exons 1, 23, 41, 44, 46, 47, 48, 49, 50, 51, 52, 53, 54, or 55 of the dystrophin gene. In embodiments, the guide RNA targets at least one of introns 44, 45, 50, 51, 52, 53, 54, or 55 of the dystrophin gene. In preferred embodiments, the guide RNAs are designed to induce skipping of exon 51 or exon 23. In embodiments, the gRNA is targeted to a splice acceptor site of exon 51 or exon 23.

Suitable gRNAs for use in the methods and compositions disclosed herein are provided as SEQ ID NOs. 60-382. (Table E). In preferred embodiments, the gRNA is selected from any one of SEQ ID No. 60 to SEQ ID No. 382.

In some embodiments, gRNAs of the disclosure comprise a sequence that is complementary to a target sequence within a coding sequence or a non-coding sequence corresponding to the DMD gene, and, therefore, hybridize to the target sequence. In some embodiments, gRNAs for Cpf1 comprise a single crRNA containing a direct repeat scaffold sequence followed by 24 nucleotides of guide sequence. In some embodiments, a “guide” sequence of the crRNA comprises a sequence of the gRNA that is complementary to a target sequence. In some embodiments, crRNA of the disclosure comprises a sequence of the gRNA that is not complementary to a target sequence. “Scaffold” sequences of the disclosure link the gRNA to the Cpf1 polypeptide. “Scaffold” sequences of the disclosure are not equivalent to a tracrRNA sequence of a gRNA-Cas9 construct.

E. Cas9 versus Cpf1

Cas9 requires two RNA molecules to cut DNA while Cpf1 needs one. The proteins also cut DNA at different places, offering researchers more options when selecting an editing site. Cas9 cuts both strands in a DNA molecule at the same position, leaving behind ‘blunt’ ends. Cpf1 leaves one strand longer than the other, creating ‘sticky’ ends that are easier to work with. Cpf1 appears to be more able to insert new sequences at the cut site, compared to Cas9. Although the CRISPR/Cas9 system can efficiently disable genes, it is challenging to insert genes or generate a knock-in. Cpf1 lacks tracrRNA, utilizes a T-rich PAM and cleaves DNA via a staggered DNA DSB.

In summary, important differences between Cpf1 and Cas9 systems are that Cpf1 recognizes different PAMs, enabling new targeting possibilities, creates 4-5 nt long sticky ends, instead of blunt ends produced by Cas9, enhancing the efficiency of genetic insertions and specificity during NHEJ or HDR, and cuts target DNA further away from PAM, further away from the Cas9 cutting site, enabling new possibilities for cleaving the DNA.

| Feature | Cas9 | Cpf1 |
|---|---|---|
| Structure | Two RNA required (Or 1 fusion | One RNA required |
| | transcript (crRNA + tracrRNA = | |
| | gRNA) | |
| Cutting | Blunt end cuts | Staggered end cuts |
| mechanism | | |
| Cutting site | Proximal to recognition site | Distal from |
| | | recognition site |
| Target sites | G-rich PAM | T-rich PAM |
| Cell type | Fast growing cells, including | Non-dividing cells, |
| | cancer cells | including nerve cells |

F. CRISPR/Cpf1-Mediated Gene Editing

The first step in editing the DMD gene using CRISPR/Cpf1 is to identify the genomic target sequence. The genomic target for the gRNAs of the disclosure can be any ˜24 nucleotide DNA sequence within the dystrophin gene, provided that the sequence is unique compared to the rest of the genome. In some embodiments, the genomic target sequence is in exon 51, exon 45, exon 44, exon 53, exon 46, exon 52, exon 50, exon 43, exon 6, exon 7, exon 8, and/or exon 55 of the human dystrophin gene. In some embodiments, the genomic target sequence is a 5′ or 3′ splice site of exon 51, exon 45, exon 44, exon 53, exon 46, exon 52, exon 50, exon 43, exon 6, exon 7, exon 8, and/or exon 55 of the human dystrophin gene. In some embodiments, the genomic target sequence is an intron immediately upstream or downstream of exon 51, exon 45, exon 44, exon 53, exon 46, exon 52, exon 50, exon 43, exon 6, exon 7, exon 8, and/or exon 55 of the human dystrophin gene. Exemplary genomic target sequences can be found in Table D.

The next step in editing the DMD gene using CRISPR/Cpf1 is to identify all Protospacer Adjacent Motif (PAM) sequences within the genetic region to be targeted. Cpf1 utilizes a T-rich PAM sequence (TTTN, wherein N is any nucleotide). The target sequence must be immediately upstream of a PAM. Once all possible PAM sequences and putative target sites have been identified, the next step is to choose which site is likely to result in the most efficient on-target cleavage. The gRNA targeting sequence needs to match the target sequence, and the gRNA targeting sequence must not match additional sites within the genome. In preferred embodiments, the gRNA targeting sequence has perfect homology to the target with no homology elsewhere in the genome. In some embodiments, a given gRNA targeting sequence will have additional sites throughout the genome where partial homology exists. These sites are called “off-targets” and should be considered when designing a gRNA. In general, off-target sites are not cleaved as efficiently when mismatches occur near the PAM sequence, so gRNAs with no homology or those with mismatches close to the PAM sequence will have the highest specificity. In addition to “off-target activity”, factors that maximize cleavage of the desired target sequence (“on-target activity”) must be considered. It is known to those of skill in the art that two gRNA targeting sequences, each having 100% homology to the target DNA may not result in equivalent cleavage efficiency. In fact, cleavage efficiency may increase or decrease depending upon the specific nucleotides within the selected target sequence. Close examination of predicted on-target and off-target activity of each potential gRNA targeting sequence is necessary to design the best gRNA. Several gRNA design programs have been developed that are capable of locating potential PAM and target sequences and ranking the associated gRNAs based on their predicted on-target and off-target activity (e.g. CRISPRdirect, available at www.crispr.dbcls.jp).

The next step is to synthesize and clone desired gRNAs. Targeting oligos can be synthesized, annealed, and inserted into plasmids containing the gRNA scaffold using standard restriction-ligation cloning. However, the exact cloning strategy will depend on the gRNA vector that is chosen. The gRNAs for Cpf1 are notably simpler than the gRNAs for Cas9, and only consist of a single crRNA containing direct repeat scaffold sequence followed by ˜24 nucleotides of guide sequence. Cpf1 requires a minimum of 16 nucleotides of guide sequence to achieve detectable DNA cleavage, and a minimum of 18 nucleotides of guide sequence to achieve efficient DNA cleavage in vitro. In some embodiments, 20-24 nucleotides of guide sequence is used. The seed region of the Cpf1 gRNA is generally within the first 5 nucleotides on the 5′ end of the guide sequence. Cpf1 makes a staggered cut in the target genomic DNA. In AsCpf1 and LbCpf1, the cut occurs 19 bp after the PAM on the targeted (+) strand, and 23 bp on the other strand.

Each gRNA should then be validated in one or more target cell lines. For example, after the CRISPR and gRNA are delivered to the cell, the genomic target region may be amplified using PCR and sequenced according to methods known to those of skill in the art.

In some embodiments, gene editing may be performed in vitro or ex vivo. In some embodiments, cells are contacted in vitro or ex vivo with a Cpf1 and a gRNA that targets a dystrophin splice site. In some embodiments, the cells are contacted with one or more nucleic acids encoding the Cpf1 and the guide RNA. In some embodiments, the one or more nucleic acids are introduced into the cells using, for example, lipofection or electroporation. Gene editing may also be performed in zygotes. In embodiments, zygotes may be injected with one or more nucleic acids encoding Cpf1 and a gRNA that targets a dystrophin splice site. The zygotes may subsequently be injected into a host.

In embodiments, the Cpf1 is provided on a vector. In embodiments, the vector contains a Cpf1 sequence derived from a Lachnospiraceae bacterium. See, for example, Uniprot Accession No. A0A182DWE3; SEQ ID NO. 443. In embodiments, the vector contains a Cpf1 sequence derived from an Acidaminococcus bacterium. See, for example, Uniprot Accession No. U2UMQ6; SEQ ID NO. 442. In some embodiments, the Cpf1 sequence is codon optimized for expression in human cells. In some embodiments, the vector further contains a sequence encoding a fluorescent protein, such as GFP, which allows Cpf1-expressing cells to be sorted using fluorescence activated cell sorting (FACS). In some embodiments, the vector is a viral vector such as an adeno-associated viral vector.

In embodiments, the gRNA is provided on a vector. In some embodiments, the vector is a viral vector such as an adeno-associated viral vector. In embodiments, the Cpf1 and the guide RNA are provided on the same vector. In embodiments, the Cpf1 and the guide RNA are provided on different vectors.

In some embodiments, the cells are additionally contacted with a single-stranded DMD oligonucleotide to effect homology directed repair. In some embodiments, small INDELs restore the protein reading frame of dystrophin (“reframing” strategy). When the reframing strategy is used, the cells may be contacted with a single gRNA. In embodiments, a splice donor or splice acceptor site is disrupted, which results in exon skipping and restoration of the protein reading frame (“exon skipping” strategy). When the exon skipping strategy is used, the cells may be contacted with two or more gRNAs.

Efficiency of in vitro or ex vivo Cpf1-mediated DNA cleavage may be assessed using techniques known to those of skill in the art, such as the T7 E1 assay. Restoration of DMD expression may be confirmed using techniques known to those of skill in the art, such as RT-PCR, western blotting, and immunocytochemistry.

In some embodiments, in vitro or ex vivo gene editing is performed in a muscle or satellite cell. In some embodiments, gene editing is performed in iPSC or iCM cells. In embodiments, the iPSC cells are differentiated after gene editing. For example, the iPSC cells may be differentiated into a muscle cell or a satellite cell after editing. In embodiments, the iPSC cells are differentiated into cardiac muscle cells, skeletal muscle cells, or smooth muscle cells. In embodiments, the iPSC cells are differentiated into cardiomyocytes. iPSC cells may be induced to differentiate according to methods known to those of skill in the art.

In some embodiments, contacting the cell with the Cpf1 and the gRNA restores dystrophin expression. In embodiments, cells which have been edited in vitro or ex vivo, or cells derived therefrom, show levels of dystrophin protein that is comparable to wild type cells. In embodiments, the edited cells, or cells derived therefrom, express dystrophin at a level that is 50%, 60%, 70%, 80%, 90%, 95% or any percentage in between of wild type dystrophin expression levels. In embodiments, the cells which have been edited in vitro or ex vivo, or cells derived therefrom, have a mitochondrial number that is comparable to that of wild type cells. In embodiments the edited cells, or cells derived therefrom, have 50%, 60%, 70%, 80%, 90%, 95% or any percentage in between as many mitochondria as wild type cells. In embodiments, the edited cells, or cells derived therefrom, show an increase in oxygen consumption rate (OCR) compared to non-edited cells at baseline.

### III. NUCLEIC ACID DELIVERY

As discussed above, in certain embodiments, expression cassettes are employed to express a transcription factor product, either for subsequent purification and delivery to a cell/subject, or for use directly in a genetic-based delivery approach. Provided herein are expression vectors which contain one or more nucleic acids encoding Cpf1 and at least one DMD guide RNA that targets a dystrophin splice site. In some embodiments, a nucleic acid encoding Cpf1 and a nucleic acid encoding at least one guide RNA are provided on the same vector. In further embodiments, a nucleic acid encoding Cpf1 and a nucleic acid encoding least one guide RNA are provided on separate vectors.

Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the Cpf1 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, 3-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), E1A, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter, and the ANF promoter.

In some embodiments, the Cpf1-gRNA constructs of the disclosure are expressed by a muscle-cell specific promoter. This muscle-cell specific promoter may be constitutively active or may be an inducible promoter.

Where a cDNA insert is employed, one will typically desire to include a polyadenylation signal to effect proper polyadenylation of the gene transcript. The nature of the polyadenylation signal is not believed to be crucial to the successful practice of the methods disclosed herein, and any such sequence may be employed such as human growth hormone and SV40 polyadenylation signals. Also contemplated as an element of the expression cassette is a terminator. These elements can serve to enhance message levels and to minimize read through from the cassette into other sequences.

B. 2A Peptide

The inventor utilizes the 2A-like self-cleaving domain from the insect virus Thosea asigna (TaV 2A peptide; SEQ ID NO. 444; EGRGSLLTCGDVEENPGP). These 2A-like domains have been shown to function across Eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems have shown greater than 99% cleavage activity (Donnelly et al., 2001). Other acceptable 2A-like peptides include, but are not limited to, equine rhinitis A virus (ERAV) 2A peptide (SEQ ID NO. 445; QCTNYALLKLAGDVESNPGP), porcine teschovirus-1 (PTV1) 2A peptide (SEQ ID NO. 446; ATNFSLLKQAGDVEENPGP) and foot and mouth disease virus (FMDV) 2A peptide (SEQ ID No. 447; PVKQLLNFDLLKLAGDVESNPGP) or modified versions thereof.

In some embodiments, the 2A peptide is used to express a reporter and a Cfpl simultaneously. The reporter may be, for example, GFP.

Other self-cleaving peptides that may be used include, but are not limited to nuclear inclusion protein a (Nia) protease, a P1 protease, a 3C protease, a L protease, a 3C-like protease, or modified versions thereof.

C. Delivery of Expression Vectors

There are a number of ways in which expression vectors may introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals.

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenoviruses of the disclosure are replication defective or at least conditionally replication defective. Other than the requirement that the adenovirus vector be replication defective, or at least conditionally defective, the nature of the adenovirus vector is not believed to be crucial to the successful practice of the methods disclosed herein. The adenovirus may be of any of the 42 different known serotypes or subgroups A-F. Adenovirus type 5 of subgroup C is the preferred starting material in order to obtain the conditional replication-defective adenovirus vector for use in the present disclosure.

As stated above, the typical vector according to the present disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression and vaccine development. Animal studies suggested that recombinant adenovirus could be used for gene therapy. Studies in administering recombinant adenovirus to different tissues include trachea instillation, muscle injection, peripheral intravenous injections and stereotactic inoculation into the brain.

The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses may be used, in which biotinylated antibodies against a retroviral envelope protein and against a specific cell receptor are used. The antibodies are coupled via the biotin components by using streptavidin. Using antibodies against major histocompatibility complex class I and class II antigens, it has been demonstrated the infection of a variety of human cells that bore those surface antigens with an ecotropic virus in vitro (Roux et al., 1989).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes. Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (see, for example, Markowitz et al., 1988; Hersdorffer et al., 1990).

Other viral vectors may be employed as expression constructs in the present disclosure. Vectors derived from viruses such as vaccinia virus, adeno-associated virus (AAV) and herpesviruses may be employed. They offer several attractive features for various mammalian cells.

In embodiments, the AAV vector is replication-defector or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof. In some embodiments, the AAV vector is not an AAV9 vector.

In some embodiments, a single viral vector is used to deliver a nucleic acid encoding Cpf1 and at least one gRNA to a cell. In some embodiments, Cpf1 is provided to a cell using a first viral vector and at least one gRNA is provided to the cell using a second viral vector. In order to effect expression of sense or antisense gene constructs, the expression construct must be delivered into a cell. The cell may be a muscle cell, a satellite cell, a mesangioblast, a bone marrow derived cell, a stromal cell or a mesenchymal stem cell. In embodiments, the cell is a cardiac muscle cell, a skeletal muscle cell, or a smooth muscle cell. In embodiments, the cell is a cell in the tibialis anterior, quadriceps, soleus, diaphragm or heart. In some embodiments, the cell is an induced pluripotent stem cell (iPSC) or inner cell mass cell (iCM). In further embodiments, the cell is a human iPSC or a human iCM. In some embodiments, human iPSCs or human iCMs of the disclosure may be derived from a cultured stem cell line, an adult stem cell, a placental stem cell, or from another source of adult or embryonic stem cells that does not require the destruction of a human embryo. Delivery to a cell may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured mammalian cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments, the liposome may be complexed with a hemagglutinating virus (HVJ). This has been shown to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### IV. PHARMACEUTICAL COMPOSITIONS AND DELIVERY METHODS

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions are normally administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the Cpf1 and gRNAs described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding Cpf1 and a guide RNA that targets a dystrophin splice site are provided to a cell ex vivo before the cell is introduced or reintroduced to a patient.

### V. SEQUENCE TABLES

The following tables provide exemplary primer sequences and gRNA sequences for use in connection with the compositions and methods disclosed herein.

TABLE C
PRIMER SEQUENCES

| | Primer Name | Primer Sequence | |
|---|---|---|---|
| Cloning primers | AgeI-nLbCpf1-F1 | F | tttttttcaggttGGaccggtgccaccATGAGCAAGCTGGA (SEQ ID NO: 8) |
| for pCpf1-2A-GFP | nLbCpf1-R1 | R | TGGGGTTATAGTAGGCCATCCACTTC (SEQ ID NO: 9) |
| | nLbCpf1-F2 | F | GATGGCCTACTATAACCCCAGCG (SEQ ID NO: 10) |
| | nLbCpf1-R2 | R | GGCATAGTCGGGGACATCATATG (SEQ ID NO: 11) |
| | AgeI-nAsCpf1-F1 | F | tttttttcaggttGGaccggtgccaccATGACACAGTTCGAG (SEQ ID NO: 12) |
| | nAsCpf1-R1 | R | TCCTTCTCAGGATTGTTCAGGTCGTA (SEQ ID NO: 13) |
| | nAsCpf1-F2 | F | CTGAACAATCCTGAGAAGGAGCC (SEQ ID NO: 14) |
| | nAsCpf1-R2 | R | GGCATAGTCGGGGACATCATATG (SEQ ID NO: 11) |
| | nCpf1-2A-GFP-F | F | ATGATGTCCCCGACTATGCCgaattcGGCAGTGGAGAGGG (SEQ ID NO: 15) |
| | nCpf1-2A-GFP-R | R | AGCGAGCTCTAGttagaattcCTTGTACAG (SEQ ID NO: 16) |
| In vitro | T7-Scaffold-F | F | CACCAGCGCTGCTTAATACGACTCACTATAGGGAAAT (SEQ ID NO: 17) |
| transcription of | T7-Scaffold-R | R | AGTAGCGCTTCTAGACCCTCACTTCCTACTCAG (SEQ ID NO: 18) |
| LbCpf1 mRNA | T7-nLb-F1 | F | AGAAGAAATATAAGACTCGAGgccaccATGAGCAAGCTGGAGAAGTTTAC (SEQ ID |
| | | | NO: 19) |
| | T7-nLb-R1 | R | TGGGGTTATAGTAGGCCATCC (SEQ ID NO: 20) |
| | T7-nLB-NLS-F2 | F | GATGGCCTACTATAACCCCAGCG (SEQ ID NO: 10) |
| | T7-nLB-NLS-R2 | R | CCCGCAGAAGGCAGCGTCGACTTAGGCATAGTCGGGGACATCATATG (SEQ ID NO: |
| | | | 21) |
| | T7-nAs-F1 | F | AGAAGAAATATAAGACTCGAGgccaccATGACACAGTTCGAGGGCTTTAC (SEQ ID |
| | | | NO: 22) |
| | T7-nAs-R1 | R | TCCTTCTCAGGATTGTTCAGGTCGTA (SEQ ID NO: 13) |
| | T7-nAs-NLS-F2 | F | CTGAACAATCCTGAGAAGGAGCC (SEQ ID NO: 14) |
| | T7-nAs-NLS-R2 | R | CCCGCAGAAGGCAGCGTCGACTTAGGCATAGTCGGGGACATCATATG (SEQ ID NO: |
| | | | 21) |
| Human DMD Exon | nLb-DMD-E51-g1-Top | F | CACCGTAATTTCTACTAAGTGTAGATgCTCCTACTCAGACTGTTACTCTGTTTTTTT |
| 51 gRNA | | | (SEQ ID NO: 23) |
| | nLb-DMD-E51-g1-Bot | R | AAACAAAAAAACAGAGTAACAGTCTGAGTAGGAGcATCTACACTTAGTAGAAATTAC |
| | | | (SEQ ID NO: 24) |
| | nLb-DMD-E51-g2-Top | F | CACCGTAATTTCTACTAAGTGTAGATtaccatgtattgctaaacaaagtaTTTTTTT |
| | | | (SEQ ID NO: 25) |
| | nLb-DMD-E51-g2-Bot | R | AAACAAAAAAAtactttgtttagcaatacatggtaATCTACACTTAGTAGAAATTAC |
| | | | (SEQ ID NO: 26) |
| | nLb-DMD-E51-g3-Top | F | CACCGTAATTTCTACTAAGTGTAGATattgaagagtaacaatttgagccaTTTTTTT |
| | | | (SEQ ID NO: 27) |
| | nLb-DMD-E51-g3-Bot | R | AAACAAAAAAAtggctcaaattgttactcttcaatATCTACACTTAGTAGAAATTAC |
| | | | (SEQ ID NO: 28) |
| | nAs-DMD-E51-g1-Top | F | CACCGTAATTTCTACTCTTGTAGATgCTCCTACTCAGACTGTTACTCTGTTTTTTT |
| | | | (SEQ ID NO: 29) |
| | nAs-DMD-E51-g1-Bot | R | AAACAAAAAAACAGAGTAACAGTCTGAGTAGGAGcATCTACAAGAGTAGAAATTAC(SEQ |
| | | | ID NO: 30) |
| Human DMD Exon | DMD-E51-T7E1-F1 | F | Ttccctggcaaggtctga (SEQ ID NO: 31) |
| 51 T7E1 | DMD-E51-T7E1-R1 | R | ATCCTCAAGGTCACCCACC (SEQ ID NO: 32) |
| Human | Riken51-RT-PCR-F1 | F | CCCAGAAGAGCAAGATAAACTTGAA (SEQ ID NO: 1) |
| cardiomyocytes | Riken51-RT-PCR-R1 | R | CTCTGTTCCAAATCCTCCATTGT (SEQ ID NO: 33) |
| RT-PCR | | | |
| Human | hmtND1-qF1 | F | CGCCACATCTACCATCACCCTC (SEQ ID NO: 3) |
| cardiomyocytes | hmtND1-qR1 | R | CGGCTAGGCTAGAGGTCGCTA (SEQ ID NO: 4) |
| mtDNA copy | hLPL-gF1 | F | GAGTATGCAGAAGCCCCGAGTC (SEQ ID NO: 5) |
| number qPCR | hLPL-qR1 | R | TCAACATGCCCAACTGGTTTCTGG (SEQ ID NO: 6) |
| Mouse Dmd Exon | nLb-dmd-E23-g1-Top | F | CACCGTAATTTCTACTAAGTGTAGATaggctctgcaaagttctTTGAAAGTTTTTTT |
| 23 gRNA | | | (SEQ ID NO: 34) |
| | nLb-dmd-E23-g1-Bot | R | AAACAAAAAAACTTTCAAagaactttgcagagcctATCTACACTTAGTAGAAATTAC |
| | | | (SEQ ID NO: 35) |
| | nLb-dmd-E23-g2-Top | F | CACCGTAATTTCTACTAAGTGTAGATAAAGAGCAACAAAATGGCttcaacTTTTTTT |
| | | | (SEQ ID NO: 36) |
| | nLb-dmd-E23-g2-Bot | R | AAACAAAAAAAgttgaaGCCATTTTGTTGCTCTTTATCTACACTTAGTAGAAATTAC |
| | | | (SEQ ID NO: 37) |
| | nLb-mdmd-E23-g2-Top | F | CACCGTAATTTCTACTAAGTGTAGATAAAGAGCAATAAAATGGCttcaacTTTTTTT |
| | | | (SEQ ID NO: 38) |
| | nLb-mdmd-E23-g2-Bot | R | AAACAAAAAAAgttgaaGCCATTTTATTGCTCTTTATCTACACTTAGTAGAAATTAC |
| | | | (SEQ ID NO: 39) |
| | nLb-dmd-E23-g3-Top | F | CACCGTAATTTCTACTAAGTGTAGATAAAGAACTTTGCAGAGCctcaaaaTTTTTTT |
| | | | (SEQ ID NO: 40) |
| | nLb-dmd-E23-g3-Bot | R | AAACAAAAAAAttttgagGCTCTGCAAAGTTCTTTATCTACACTTAGTAGAAATTAC |
| | | | (SEQ ID NO: 41) |
| | nLb-dmd-I22-g1-Top | F | CACCGTAATTTCTACTAAGTGTAGATctgaatatctatgcattaataactTTTTTTT |
| | | | (SEQ ID NO: 42) |
| | nLb-dmd-I22-g1-Bot | R | AAACAAAAAAAagttattaatgcatagatattcagATCTACACTTAGTAGAAATTAC |
| | | | (SEQ ID NO: 43) |
| | nLb-dmd-I22-g2-Top | F | CACCGTAATTTCTACTAAGTGTAGATtattatattacagggcatattataTTTTTTT |
| | | | (SEQ ID NO: 44) |
| | nLb-dmd-I22-g2-Bot | R | AAACAAAAAAAtataatatgccctgtaatataataATCTACACTTAGTAGAAATTAC |
| | | | (SEQ ID NO: 45) |
| | nLb-dmd-I23-g3-Top | F | CACCGTAATTTCTACTAAGTGTAGATAGgtaagccgaggtttggcctttaTTTTTTT |
| | | | (SEQ ID NO: 46) |
| | nLb-dmd-I23-g3-Bot | R | AAACAAAAAAAtaaaggccaaacctcggcttacCTATCTACACTTAGTAGAAATTAC |
| | | | (SEQ ID NO: 47) |
| | nLb-dmd-I23-g4-Top | F | CACCGTAATTTCTACTAAGTGTAGATcccagagtccttcaaagatattgaTTTTTTT |
| | | | (SEQ ID NO: 48) |
| | nLb-dmd-I23-g4-Bot | R | AAACAAAAAAAtcaatatctttgaaggactctgggATCTACACTTAGTAGAAATTAC |
| | | | (SEQ ID NO: 49) |
| In vitro | T7-Lb-dmd-E23-uF | F | GAATTGTAATACGACTCACTATAGGGTAATTTCTACTAAGTGTAGAT (SEQ ID NO: |
| transcription | | | 50) |
| of LbCpf1 gRNA | T7-Lb-dmd-E23-g1-R | R | CTTTCAAagaactttgcagagcctATCTACACTTAGTAGAAATTA (SEQ ID NO: 51) |
| | T7-Lb-dmd-E23-mg2-R | R | GttgaaGCCATTTTATTGCTCTTTATCTACACTTAGTAGAAATTA (SEQ ID NO: 52) |
| | T7-Lb-dmd-E23-g3-R | R | ttttgagGCTCTGCAAAGTTCTTTATCTACACTTAGTAGAAATTA (SEQ ID NO: 53) |
| | T7-Lb-dmd-I22-g2-R | R | tataatatgccctgtaatataataATCTACACTTAGTAGAAATTACCCTATAGTGAG |
| | | | (SEQ ID NO: 54) |
| | T7-Lb-dmd-I22-g4-R | R | tcaatatctttgaaggactctgggATCTACACTTAGTAGAAATTACCCTATAGTGAG |
| | | | (SEQ ID NO: 55) |
| Mouse Dmd Exon | Dmd-E23-T7E1-F729 | F | Gagaaacttctgtgatgtgaggacata (SEQ ID NO: 56) |
| 23 T7E1 | Dmd-E23-T7E1-R1 | R | CAAACCTCGGCTTACCTGAAAT (SEQ ID NO: 57) |
| | Dmd-E23-T7E1-R729 | R | Caatatctttgaaggactctgggtaaa (SEQ ID NO: 58) |
| | Dmd-E23-T7E1-R3 | R | Aattaatagaagtcaatgtagggaagg (SEQ ID NO: 59) |

TABLE D
Genomic Target Sequences

| Targeted gRNA | Guide | | | | SEQ ID |
|---|---|---|---|---|---|
| Exon | # | Strand | Genomic Target Sequence* | PAM | NO. |
| Human-Exon 51 | 4 | 1 | tctttttcttcttttttccttttt | tttt | 60 |
| Human-Exon 51 | 5 | 1 | ctttttcttcttttttcctttttG | tttt | 61 |
| Human-Exon 51 | 6 | 1 | tttttcttcttttttcctttttGC | tttc | 62 |
| Human-Exon 51 | 7 | 1 | tcttcttttttcctttttGCAAAA | tttt | 63 |
| Human-Exon 51 | 8 | 1 | cttcttttttcctttttGCAAAAA | tttt | 64 |
| Human-Exon 51 | 9 | 1 | ttcttttttcctttttGCAAAAAC | tttc | 65 |
| Human-Exon 51 | 10 | 1 | ttcctttttGCAAAAACCCAAAAT | tttt | 66 |
| Human-Exon 51 | 11 | 1 | tcctttttGCAAAAACCCAAAATA | tttt | 67 |
| Human-Exon 51 | 12 | 1 | cctttttGCAAAAACCCAAAATAT | tttt | 68 |
| Human-Exon 51 | 13 | 1 | ctttttGCAAAAACCCAAAATATT | tttc | 69 |
| Human-Exon 51 | 14 | 1 | tGCAAAAACCCAAAATATTTTAGC | tttt | 70 |
| Human-Exon 51 | 15 | 1 | GCAAAAACCCAAAATATTTTAGCT | tttt | 71 |
| Human-Exon 51 | 16 | 1 | CAAAAACCCAAAATATTTTAGCTC | tttG | 72 |
| Human-Exon 51 | 17 | 1 | AGCTCCTACTCAGACTGTTACTCT | TTTT | 73 |
| Human-Exon 51 | 18 | 1 | GCTCCTACTCAGACTGTTACTCTG | TTTA | 74 |
| Human-Exon 51 | 19 | −1 | CTTAGTAACCACAGGTTGTGTCAC | TTTC | 75 |
| Human-Exon 51 | 20 | −1 | GAGATGGCAGTTTCCTTAGTAACC | TTTG | 76 |
| Human-Exon 51 | 21 | −1 | TAGTTTGGAGATGGCAGTTTCCTT | TTTC | 77 |
| Human-Exon 51 | 22 | −1 | TTCTCATACCTTCTGCTTGATGAT | TTTT | 78 |
| Human-Exon 51 | 23 | −1 | TCATTTTTTCTCATACCTTCTGCT | TTTA | 79 |
| Human-Exon 51 | 24 | −1 | ATCATTTTTTCTCATACCTTCTGC | TTTT | 80 |
| Human-Exon 51 | 25 | −1 | AAGAAAAACTTCTGCCAACTTTTA | TTTA | 81 |
| Human-Exon 51 | 26 | −1 | AAAGAAAAACTTCTGCCAACTTTT | TTTT | 82 |
| Human-Exon 51 | 27 | 1 | TCTTTAAAATGAAGATTTTCCACC | TTTT | 83 |
| Human-Exon 51 | 28 | 1 | CTTTAAAATGAAGATTTTCCACCA | TTTT | 84 |
| Human-Exon 51 | 29 | 1 | TTTAAAATGAAGATTTTCCACCAA | TTTC | 85 |
| Human-Exon 51 | 30 | 1 | AAATGAAGATTTTCCACCAATCAC | TTTA | 86 |
| Human-Exon 51 | 31 | 1 | CCACCAATCACTTTACTCTCCTAG | TTTT | 87 |
| Human-Exon 51 | 32 | 1 | CACCAATCACTTTACTCTCCTAGA | TTTC | 88 |
| Human-Exon 51 | 33 | 1 | CTCTCCTAGACCATTTCCCACCAG | TTTA | 89 |
| Human-Exon 45 | 1 | −1 | agaaaagattaaacagtgtgctac | tttg | 90 |
| Human-Exon 45 | 2 | −1 | tttgagaaaagattaaacagtgtg | TTTa | 91 |
| Human-Exon 45 | 3 | −1 | atttgagaaaagattaaacagtgt | TTTT | 92 |
| Human-Exon 45 | 4 | −1 | Tatttgagaaaagattaaacagtg | TTTT | 93 |
| Human-Exon 45 | 5 | 1 | atcttttctcaaatAAAAAGACAT | ttta | 94 |
| Human-Exon 45 | 6 | 1 | ctcaaatAAAAAGACATGGGGCTT | tttt | 95 |
| Human-Exon 45 | 7 | 1 | tcaaatAAAAAGACATGGGGCTTC | tttc | 96 |
| Human-Exon 45 | 8 | 1 | TGTTTTGCCTTTTTGGTATCTTAC | TTTT | 97 |
| Human-Exon 45 | 9 | 1 | GTTTTGCCTTTTTGGTATCTTACA | TTTT | 98 |
| Human-Exon 45 | 10 | 1 | TTTTGCCTTTTTGGTATCTTACAG | TTTG | 99 |
| Human-Exon 45 | 11 | 1 | GCCTTTTTGGTATCTTACAGGAAC | TTTT | 100 |
| Human-Exon 45 | 12 | 1 | CCTTTTTGGTATCTTACAGGAACT | TTTG | 101 |
| Human-Exon 45 | 13 | 1 | TGGTATCTTACAGGAACTCCAGGA | TTTT | 102 |
| Human-Exon 45 | 14 | 1 | GGTATCTTACAGGAACTCCAGGAT | TTTT | 103 |
| Human-Exon 45 | 15 | −1 | AGGATTGCTGAATTATTTCTTCCC | TTTG | 104 |
| Human-Exon 45 | 16 | −1 | GAGGATTGCTGAATTATTTCTTCC | TTTT | 105 |
| Human-Exon 45 | 17 | −1 | TGAGGATTGCTGAATTATTTCTTC | TTTT | 106 |
| Human-Exon 45 | 18 | −1 | CTGTAGAATACTGGCATCTGTTTT | TTTC | 107 |
| Human-Exon 45 | 19 | −1 | CCTGTAGAATACTGGCATCTGTTT | TTTT | 108 |
| Human-Exon 45 | 20 | −1 | TCCTGTAGAATACTGGCATCTGTT | TTTT | 109 |
| Human-Exon 45 | 21 | −1 | CAGACCTCCTGCCACCGCAGATTC | TTTG | 110 |
| Human-Exon 45 | 22 | −1 | TGTCTGACAGCTGTTTGCAGACCT | TTTC | 111 |
| Human-Exon 45 | 23 | −1 | CTGTCTGACAGCTGTTTGCAGACC | TTTT | 112 |
| Human-Exon 45 | 24 | −1 | TCTGTCTGACAGCTGTTTGCAGAC | TTTT | 113 |
| Human-Exon 45 | 25 | −1 | TTCTGTCTGACAGCTGTTTGCAGA | TTTT | 114 |
| Human-Exon 45 | 26 | −1 | ATTCCTATTAGATCTGTCGCCCTA | TTTC | 115 |
| Human-Exon 45 | 27 | −1 | CATTCCTATTAGATCTGTCGCCCT | TTTT | 116 |
| Human-Exon 45 | 28 | 1 | AGCAGACTTTTTAAGCTTTCTTTA | TTTT | 117 |
| Human-Exon 45 | 29 | 1 | GCAGACTTTTTAAGCTTTCTTTAG | TTTA | 118 |
| Human-Exon 45 | 30 | 1 | TAAGCTTTCTTTAGAAGAATATTT | TTTT | 119 |
| Human-Exon 45 | 31 | 1 | AAGCTTTCTTTAGAAGAATATTTC | TTTT | 120 |
| Human-Exon 45 | 32 | 1 | AGCTTTCTTTAGAAGAATATTTCA | TTTA | 121 |
| Human-Exon 45 | 33 | 1 | TTTAGAAGAATATTTCATGAGAGA | TTTC | 122 |
| Human-Exon 45 | 34 | 1 | GAAGAATATTTCATGAGAGATTAT | TTTA | 123 |
| Human-Exon 44 | 1 | 1 | TCAGTATAACCAAAAAATATACGC | TTTG | 124 |
| Human-Exon 44 | 2 | 1 | acataatccatctatttttcttga | tttt | 125 |
| Human-Exon 44 | 3 | 1 | cataatccatctatttttcttgat | ttta | 126 |
| Human-Exon 44 | 4 | 1 | tcttgatccatatgcttttACCTG | tttt | 127 |
| Human-Exon 44 | 5 | 1 | cttgatccatatgcttttACCTGC | tttt | 128 |
| Human-Exon 44 | 6 | 1 | ttgatccatatgcttttACCTGCA | tttc | 129 |
| Human-Exon 44 | 7 | −1 | TCAACAGATCTGTCAAATCGCCTG | TTTC | 130 |
| Human-Exon 44 | 8 | 1 | ACCTGCAGGCGATTTGACAGATCT | tttt | 131 |
| Human-Exon 44 | 9 | 1 | CCTGCAGGCGATTTGACAGATCTG | tttA | 132 |
| Human-Exon 44 | 10 | 1 | ACAGATCTGTTGAGAAATGGCGGC | TTTG | 133 |
| Human-Exon 44 | 11 | −1 | TATCATAATGAAAACGCCGCCATT | TTTA | 134 |
| Human-Exon 44 | 12 | 1 | CATTATGATATAAAGATATTTAAT | TTTT | 135 |
| Human-Exon 44 | 13 | −1 | TATTTAGCATGTTCCCAATTCTCA | TTTG | 136 |
| Human-Exon 44 | 14 | −1 | GAAAAAACAAATCAAAGACTTACC | TTTC | 137 |
| Human-Exon 44 | 15 | 1 | ATTTGTTTTTTCGAAATTGTATTT | TTTG | 138 |
| Human-Exon 44 | 16 | 1 | TTTTTTCGAAATTGTATTTATCTT | TTTG | 139 |
| Human-Exon 44 | 17 | 1 | TTCGAAATTGTATTTATCTTCAGC | TTTT | 140 |
| Human-Exon 44 | 18 | 1 | TCGAAATTGTATTTATCTTCAGCA | TTTT | 141 |
| Human-Exon 44 | 19 | 1 | CGAAATTGTATTTATCTTCAGCAC | TTTT | 142 |
| Human-Exon 44 | 20 | 1 | GAAATTGTATTTATCTTCAGCACA | TTTC | 143 |
| Human-Exon 44 | 21 | −1 | AGAAGTTAAAGAGTCCAGATGTGC | TTTA | 144 |
| Human-Exon 44 | 22 | 1 | TCTTCAGCACATCTGGACTCTTTA | TTTA | 145 |
| Human-Exon 44 | 23 | −1 | CATCACCCTTCAGAACCTGATCTT | TTTC | 146 |
| Human-Exon 44 | 24 | 1 | ACTTCTTAAAGATCAGGTTCTGAA | TTTA | 147 |
| Human-Exon 44 | 25 | 1 | GACTGTTGTTGTCATCATTATATT | TTTT | 148 |
| Human-Exon 44 | 26 | 1 | ACTGTTGTTGTCATCATTATATTA | TTTG | 149 |
| Human-Exon 53 | 1 | −1 | AACTAGAATAAAAGGAAAAATAAA | TTTC | 150 |
| Human-Exon 53 | 2 | 1 | CTACTATATATTTATTTTTCCTTT | TTTA | 151 |
| Human-Exon 53 | 3 | 1 | TTTTTCCTTTTATTCTAGTTGAAA | TTTA | 152 |
| Human-Exon 53 | 4 | 1 | TCCTTTTATTCTAGTTGAAAGAAT | TTTT | 153 |
| Human-Exon 53 | 5 | 1 | CCTTTTATTCTAGTTGAAAGAATT | TTTT | 154 |
| Human-Exon 53 | 6 | 1 | CTTTTATTCTAGTTGAAAGAATTC | TTTC | 155 |
| Human-Exon 53 | 7 | 1 | ATTCTAGTTGAAAGAATTCAGAAT | TTTT | 156 |
| Human-Exon 53 | 8 | 1 | TTCTAGTTGAAAGAATTCAGAATC | TTTA | 157 |
| Human-Exon 53 | 9 | −1 | ATTCAACTGTTGCCTCCGGTTCTG | TTTC | 158 |
| Human-Exon 53 | 10 | −1 | ACATTTCATTCAACTGTTGCCTCC | TTTA | 159 |
| Human-Exon 53 | 11 | −1 | CTTTTGGATTGCATCTACTGTATA | TTTT | 160 |
| Human-Exon 53 | 12 | −1 | TGTGATTTTCTTTTGGATTGCATC | TTTC | 161 |
| Human-Exon 53 | 13 | −1 | ATACTAACCTTGGTTTCTGTGATT | TTTG | 162 |
| Human-Exon 53 | 14 | −1 | AAAAGGTATCTTTGATACTAACCT | TTTA | 163 |
| Human-Exon 53 | 15 | −1 | AAAAAGGTATCTTTGATACTAACC | TTTT | 164 |
| Human-Exon 53 | 16 | −1 | TTTTAAAAAGGTATCTTTGATACT | TTTA | 165 |
| Human-Exon 53 | 17 | −1 | ATTTTAAAAAGGTATCTTTGATAC | TTTT | 166 |
| Human-Exon 46 | 1 | −1 | TTAATGCAAACTGGGACACAAACA | TTTG | 167 |
| Human-Exon 46 | 2 | 1 | TAAATTGCCATGTTTGTGTCCCAG | TTTT | 168 |
| Human-Exon 46 | 3 | 1 | AAATTGCCATGTTTGTGTCCCAGT | TTTT | 169 |
| Human-Exon 46 | 4 | 1 | AATTGCCATGTTTGTGTCCCAGTT | TTTA | 170 |
| Human-Exon 46 | 5 | 1 | TGTCCCAGTTTGCATTAACAAATA | TTTG | 171 |
| Human-Exon 46 | 6 | −1 | CAACATAGTTCTCAAACTATTTGT | tttC | 172 |
| Human-Exon 46 | 7 | −1 | CCAACATAGTTCTCAAACTATTTG | tttt | 173 |
| Human-Exon 46 | 8 | −1 | tCCAACATAGTTCTCAAACTATTT | tttt | 174 |
| Human-Exon 46 | 9 | −1 | tttCCAACATAGTTCTCAAACTAT | tttt | 175 |
| Human-Exon 46 | 10 | −1 | ttttCCAACATAGTTCTCAAACTA | tttt | 176 |
| Human-Exon 46 | 11 | −1 | tttttCCAACATAGTTCTCAAACT | tttt | 177 |
| Human-Exon 46 | 12 | 1 | CATTAACAAATAGTTTGAGAACTA | TTTG | 178 |
| Human-Exon 46 | 13 | 1 | AGAACTATGTTGGaaaaaaaaaTA | TTTG | 179 |
| Human-Exon 46 | 14 | −1 | GTTCTTCTAGCCTGGAGAAAGAAG | TTTT | 180 |
| Human-Exon 46 | 15 | 1 | ATTCTTCTTTCTCCAGGCTAGAAG | TTTT | 181 |
| Human-Exon 46 | 16 | 1 | TTCTTCTTTCTCCAGGCTAGAAGA | TTTA | 182 |
| Human-Exon 46 | 17 | 1 | TCCAGGCTAGAAGAACAAAAGAAT | TTTC | 183 |
| Human-Exon 46 | 18 | −1 | AAATTCTGACAAGATATTCTTTTG | TTTG | 184 |
| Human-Exon 46 | 19 | −1 | CTTTTAGTTGCTGCTCTTTTCCAG | TTTT | 185 |
| Human-Exon 46 | 20 | −1 | AGAAAATAAAATTACCTTGACTTG | TTTG | 186 |
| Human-Exon 46 | 21 | −1 | TGCAAGCAGGCCCTGGGGGATTTG | TTTA | 187 |
| Human-Exon 46 | 22 | 1 | ATTTTCTCAAATCCCCCAGGGCCT | TTTT | 188 |
| Human-Exon 46 | 23 | 1 | TTTTCTCAAATCCCCCAGGGCCTG | TTTA | 189 |
| Human-Exon 46 | 24 | 1 | CTCAAATCCCCCAGGGCCTGCTTG | TTTT | 190 |
| Human-Exon 46 | 25 | 1 | TCAAATCCCCCAGGGCCTGCTTGC | TTTC | 191 |
| Human-Exon 46 | 26 | 1 | TTAATTCAATCATTGGTTTTCTGC | TTTT | 192 |
| Human-Exon 46 | 27 | 1 | TAATTCAATCATTGGTTTTCTGCC | TTTT | 193 |
| Human-Exon 46 | 28 | 1 | AATTCAATCATTGGTTTTCTGCCC | TTTT | 194 |
| Human-Exon 46 | 29 | 1 | ATTCAATCATTGGTTTTCTGCCCA | TTTA | 195 |
| Human-Exon 46 | 30 | −1 | GCAAGGAACTATGAATAACCTAAT | TTTA | 196 |
| Human-Exon 46 | 31 | 1 | CTGCCCATTAGGTTATTCATAGTT | TTTT | 197 |
| Human-Exon 46 | 32 | 1 | TGCCCATTAGGTTATTCATAGTTC | TTTC | 198 |
| Human-Exon 52 | 1 | −1 | TAGAAAACAATTTAACAGGAAATA | TTTA | 199 |
| Human-Exon 52 | 2 | 1 | CTGTTAAATTGTTTTCTATAAACC | TTTC | 200 |
| Human-Exon 52 | 3 | −1 | GAAATAAAAAAGATGTTACTGTAT | TTTA | 201 |
| Human-Exon 52 | 4 | −1 | AGAAATAAAAAAGATGTTACTGTA | TTTT | 202 |
| Human-Exon 52 | 5 | 1 | CTATAAACCCTTATACAGTAACAT | TTTT | 203 |
| Human-Exon 52 | 6 | 1 | TATAAACCCTTATACAGTAACATC | TTTC | 204 |
| Human-Exon 52 | 7 | 1 | TTATTTCTAAAAGTGTTTTGGCTG | TTTT | 205 |
| Human-Exon 52 | 8 | 1 | TATTTCTAAAAGTGTTTTGGCTGG | TTTT | 206 |
| Human-Exon 52 | 9 | 1 | ATTTCTAAAAGTGTTTTGGCTGGT | TTTT | 207 |
| Human-Exon 52 | 10 | 1 | TTTCTAAAAGTGTTTTGGCTGGTC | TTTA | 208 |
| Human-Exon 52 | 11 | 1 | TAAAAGTGTTTTGGCTGGTCTCAC | TTTC | 209 |
| Human-Exon 52 | 12 | −1 | CATAATACAAAGTAAAGTACAATT | TTTA | 210 |
| Human-Exon 52 | 13 | −1 | ACATAATACAAAGTAAAGTACAAT | TTTT | 211 |
| Human-Exon 52 | 14 | 1 | GGCTGGTCTCACAATTGTACTTTA | TTTT | 212 |
| Human-Exon 52 | 15 | 1 | GCTGGTCTCACAATTGTACTTTAC | TTTG | 213 |
| Human-Exon 52 | 16 | 1 | CTTTGTATTATGTAAAAGGAATAC | TTTA | 214 |
| Human-Exon 52 | 17 | 1 | TATTATGTAAAAGGAATACACAAC | TTTG | 215 |
| Human-Exon 52 | 18 | 1 | TTCTTACAGGCAACAATGCAGGAT | TTTG | 216 |
| Human-Exon 52 | 19 | 1 | GAACAGAGGCGTCCCCAGTTGGAA | TTTG | 217 |
| Human-Exon 52 | 20 | −1 | GGCAGCGGTAATGAGTTCTTCCAA | TTTG | 218 |
| Human-Exon 52 | 21 | −1 | TCAAATTTTGGGCAGCGGTAATGA | TTTT | 219 |
| Human-Exon 52 | 22 | 1 | AAAAACAAGACCAGCAATCAAGAG | TTTG | 220 |
| Human-Exon 52 | 23 | −1 | TGTGTCCCATGCTTGTTAAAAAAC | TTTG | 221 |
| Human-Exon 52 | 24 | 1 | TTAACAAGCATGGGACACACAAAG | TTTT | 222 |
| Human-Exon 52 | 25 | 1 | TAACAAGCATGGGACACACAAAGC | TTTT | 223 |
| Human-Exon 52 | 26 | 1 | AACAAGCATGGGACACACAAAGCA | TTTT | 224 |
| Human-Exon 52 | 27 | 1 | ACAAGCATGGGACACACAAAGCAA | TTTA | 225 |
| Human-Exon 52 | 28 | −1 | TTGAAACTTGTCATGCATCTTGCT | TTTA | 226 |
| Human-Exon 52 | 29 | −1 | ATTGAAACTTGTCATGCATCTTGC | TTTT | 227 |
| Human-Exon 52 | 30 | −1 | TATTGAAACTTGTCATGCATCTTG | TTTT | 228 |
| Human-Exon 52 | 31 | 1 | AATAAAAACTTAAGTTCATATATC | TTTC | 229 |
| Human-Exon 50 | 1 | −1 | GTGAATATATTATTGGATTTCTAT | TTTG | 230 |
| Human-Exon 50 | 2 | −1 | AAGATAATTCATGAACATCTTAAT | TTTG | 231 |
| Human-Exon 50 | 3 | −1 | ACAGAAAAGCATACACATTACTTA | TTTA | 232 |
| Human-Exon 50 | 4 | 1 | CTGTTAAAGAGGAAGTTAGAAGAT | TTTT | 233 |
| Human-Exon 50 | 5 | 1 | TGTTAAAGAGGAAGTTAGAAGATC | TTTC | 234 |
| Human-Exon 50 | 6 | −1 | CCGCCTTCCACTCAGAGCTCAGAT | TTTA | 235 |
| Human-Exon 50 | 7 | −1 | CCCTCAGCTCTTGAAGTAAACGGT | TTTG | 236 |
| Human-Exon 50 | 8 | 1 | CTTCAAGAGCTGAGGGCAAAGCAG | TTTA | 237 |
| Human-Exon 50 | 9 | −1 | AACAAATAGCTAGAGCCAAAGAGA | TTTG | 238 |
| Human-Exon 50 | 10 | −1 | GAACAAATAGCTAGAGCCAAAGAG | TTTT | 239 |
| Human-Exon 50 | 11 | 1 | GCTCTAGCTATTTGTTCAAAAGTG | TTTG | 240 |
| Human-Exon 50 | 12 | 1 | TTCAAAAGTGCAACTATGAAGTGA | TTTG | 241 |
| Human-Exon 50 | 13 | −1 | TCTCTCACCCAGTCATCACTTCAT | TTTC | 242 |
| Human-Exon 50 | 14 | −1 | CTCTCTCACCCAGTCATCACTTCA | TTTT | 243 |
| Human-Exon 43 | 1 | 1 | tatatatatatatatTTTTCTCTT | TTTG | 244 |
| Human-Exon 43 | 2 | 1 | TCTCTTTCTATAGACAGCTAATTC | tTTT | 245 |
| Human-Exon 43 | 3 | 1 | CTCTTTCTATAGACAGCTAATTCA | TTTT | 246 |
| Human-Exon 43 | 4 | −1 | AAACAGTAAAAAAATGAATTAGCT | TTTA | 247 |
| Human-Exon 43 | 5 | 1 | TCTTTCTATAGACAGCTAATTCAT | TTTC | 248 |
| Human-Exon 43 | 6 | −1 | AAAACAGTAAAAAAATGAATTAGC | TTTT | 249 |
| Human-Exon 43 | 7 | 1 | TATAGACAGCTAATTCATTTTTTT | TTTC | 250 |
| Human-Exon 43 | 8 | −1 | TATTCTGTAATATAAAAATTTTAA | TTTA | 251 |
| Human-Exon 43 | 9 | −1 | ATATTCTGTAATATAAAAATTTTA | TTTT | 252 |
| Human-Exon 43 | 10 | 1 | TTTACTGTTTTAAAATTTTTATAT | TTTT | 253 |
| Human-Exon 43 | 11 | 1 | TTACTGTTTTAAAATTTTTATATT | TTTT | 254 |
| Human-Exon 43 | 12 | 1 | TACTGTTTTAAAATTTTTATATTA | TTTT | 255 |
| Human-Exon 43 | 13 | 1 | ACTGTTTTAAAATTTTTATATTAC | TTTT | 256 |
| Human-Exon 43 | 14 | 1 | CTGTTTTAAAATTTTTATATTACA | TTTA | 257 |
| Human-Exon 43 | 15 | 1 | AAAATTTTTATATTACAGAATATA | TTTT | 258 |
| Human-Exon 43 | 16 | 1 | AAATTTTTATATTACAGAATATAA | TTTA | 259 |
| Human-Exon 43 | 17 | −1 | TTGTAGACTATCTTTTATATTCTG | TTTG | 260 |
| Human-Exon 43 | 18 | 1 | TATATTACAGAATATAAAAGATAG | TTTT | 261 |
| Human-Exon 43 | 19 | 1 | ATATTACAGAATATAAAAGATAGT | TTTT | 262 |
| Human-Exon 43 | 20 | 1 | TATTACAGAATATAAAAGATAGTC | TTTA | 263 |
| Human-Exon 43 | 21 | −1 | CAATGCTGCTGTCTTCTTGCTATG | TTTG | 264 |
| Human-Exon 43 | 22 | 1 | CAATGGGAAAAAGTTAACAAAATG | TTTC | 265 |
| Human-Exon 43 | 23 | −1 | TGCAAGTATCAAGAAAAATATATG | TTTC | 266 |
| Human-Exon 43 | 24 | 1 | TCTTGATACTTGCAGAAATGATTT | TTTT | 267 |
| Human-Exon 43 | 25 | 1 | CTTGATACTTGCAGAAATGATTTG | TTTT | 268 |
| Human-Exon 43 | 26 | 1 | TTGATACTTGCAGAAATGATTTGT | TTTC | 269 |
| Human-Exon 43 | 27 | 1 | TTTTCAGGGAACTGTAGAATTTAT | TTTG | 270 |
| Human-Exon 43 | 28 | −1 | CATGGAGGGTACTGAAATAAATTC | TTTC | 271 |
| Human-Exon 43 | 29 | −1 | CCATGGAGGGTACTGAAATAAATT | TTTT | 272 |
| Human-Exon 43 | 30 | 1 | CAGGGAACTGTAGAATTTATTTCA | TTTT | 273 |
| Human-Exon 43 | 31 | −1 | TCCATGGAGGGTACTGAAATAAAT | TTTT | 274 |
| Human-Exon 43 | 32 | 1 | AGGGAACTGTAGAATTTATTTCAG | TTTC | 275 |
| Human-Exon 43 | 33 | −1 | TTCCATGGAGGGTACTGAAATAAA | TTTT | 276 |
| Human-Exon 43 | 34 | −1 | CCTGTCTTTTTTCCATGGAGGGTA | TTTC | 277 |
| Human-Exon 43 | 35 | −1 | CCCTGTCTTTTTTCCATGGAGGGT | TTTT | 278 |
| Human-Exon 43 | 36 | −1 | TCCCTGTCTTTTTTCCATGGAGGG | TTTT | 279 |
| Human-Exon 43 | 37 | 1 | TTTCAGTACCCTCCATGGAAAAAA | TTTA | 280 |
| Human-Exon 43 | 38 | 1 | AGTACCCTCCATGGAAAAAAGACA | TTTC | 281 |
| Human-Exon 6 | 1 | 1 | AGTTTGCATGGTTCTTGCTCAAGG | TTTA | 282 |
| Human-Exon 6 | 2 | −1 | ATAAGAAAATGCATTCCTTGAGCA | TTTC | 283 |
| Human-Exon 6 | 3 | −1 | CATAAGAAAATGCATTCCTTGAGC | TTTT | 284 |
| Human-Exon 6 | 4 | 1 | CATGGTTCTTGCTCAAGGAATGCA | TTTG | 285 |
| Human-Exon 6 | 5 | −1 | ACCTACATGTGGAAATAAATTTTC | TTTG | 286 |
| Human-Exon 6 | 6 | −1 | GACCTACATGTGGAAATAAATTTT | TTTT | 287 |
| Human-Exon 6 | 7 | −1 | TGACCTACATGTGGAAATAAATTT | TTTT | 288 |
| Human-Exon 6 | 8 | 1 | CTTATGAAAATTTATTTCCACATG | TTTT | 289 |
| Human-Exon 6 | 9 | 1 | TTATGAAAATTTATTTCCACATGT | TTTC | 290 |
| Human-Exon 6 | 10 | −1 | ATTACATTTTTGACCTACATGTGG | TTTC | 291 |
| Human-Exon 6 | 11 | −1 | CATTACATTTTTGACCTACATGTG | TTTT | 292 |
| Human-Exon 6 | 12 | −1 | TCATTACATTTTTGACCTACATGT | TTTT | 293 |
| Human-Exon 6 | 13 | 1 | TTTCCACATGTAGGTCAAAAATGT | TTTA | 294 |
| Human-Exon 6 | 14 | 1 | CACATGTAGGTCAAAAATGTAATG | TTTC | 295 |
| Human-Exon 6 | 15 | −1 | TTGCAATCCAGCCATGATATTTTT | TTTG | 296 |
| Human-Exon 6 | 16 | −1 | ACTGTTGGTTTGTTGCAATCCAGC | TTTC | 297 |
| Human-Exon 6 | 17 | −1 | CACTGTTGGTTTGTTGCAATCCAG | TTTT | 298 |
| Human-Exon 6 | 18 | 1 | AATGCTCTCATCCATAGTCATAGG | TTTG | 299 |
| Human-Exon 6 | 19 | −1 | ATGTCTCAGTAATCTTCTTACCTA | TTTA | 300 |
| Human-Exon 6 | 20 | −1 | CAAGTTATTTAATGTCTCAGTAAT | TTTA | 301 |
| Human-Exon 6 | 21 | −1 | ACAAGTTATTTAATGTCTCAGTAA | TTTT | 302 |
| Human-Exon 6 | 22 | 1 | GACTCTGATGACATATTTTTCCCC | TTTA | 303 |
| Human-Exon 6 | 23 | 1 | TCCCCAGTATGGTTCCAGATCATG | TTTT | 304 |
| Human-Exon 6 | 24 | 1 | CCCCAGTATGGTTCCAGATCATGT | TTTT | 305 |
| Human-Exon 6 | 25 | 1 | CCCAGTATGGTTCCAGATCATGTC | TTTC | 306 |
| Human-Exon 7 | 1 | 1 | TATTTGTCTTtgtgtatgtgtgta | TTTA | 307 |
| Human-Exon 7 | 2 | 1 | TCTTtgtgtatgtgtgtatgtgta | TTTG | 308 |
| Human-Exon 7 | 3 | 1 | tgtatgtgtgtatgtgtatgtgtt | TTtg | 309 |
| Human-Exon 7 | 4 | 1 | AGGCCAGACCTATTTGACTGGAAT | ttTT | 310 |
| Human-Exon 7 | 5 | 1 | GGCCAGACCTATTTGACTGGAATA | tTTA | 311 |
| Human-Exon 7 | 6 | 1 | ACTGGAATAGTGTGGTTTGCCAGC | TTTG | 312 |
| Human-Exon 7 | 7 | 1 | CCAGCAGTCAGCCACACAACGACT | TTTG | 313 |
| Human-Exon 7 | 8 | −1 | TCTATGCCTAATTGATATCTGGCG | TTTC | 314 |
| Human-Exon 7 | 9 | −1 | CCAACCTTCAGGATCGAGTAGTTT | TTTA | 315 |
| Human-Exon 7 | 10 | 1 | TGGACTACCACTGCTTTTAGTATG | TTTC | 316 |
| Human-Exon 7 | 11 | 1 | AGTATGGTAGAGTTTAATGTTTTC | TTTT | 317 |
| Human-Exon 7 | 12 | 1 | GTATGGTAGAGTTTAATGTTTTCA | TTTA | 318 |
| Human-Exon 8 | 1 | −1 | AGACTCTAAAAGGATAATGAACAA | TTTG | 319 |
| Human-Exon 8 | 2 | 1 | ACTTTGATTTGTTCATTATCCTTT | TTTA | 320 |
| Human-Exon 8 | 3 | −1 | TATATTTGAGACTCTAAAAGGATA | TTTC | 321 |
| Human-Exon 8 | 4 | 1 | ATTTGTTCATTATCCTTTTAGAGT | TTTG | 322 |
| Human-Exon 8 | 5 | −1 | GTTTCTATATTTGAGACTCTAAAA | TTTG | 323 |
| Human-Exon 8 | 6 | −1 | GGTTTCTATATTTGAGACTCTAAA | TTTT | 324 |
| Human-Exon 8 | 7 | −1 | TGGTTTCTATATTTGAGACTCTAA | TTTT | 325 |
| Human-Exon 8 | 8 | 1 | TTCATTATCCTTTTAGAGTCTCAA | TTTG | 326 |
| Human-Exon 8 | 9 | 1 | AGAGTCTCAAATATAGAAACCAAA | TTTT | 327 |
| Human-Exon 8 | 10 | 1 | GAGTCTCAAATATAGAAACCAAAA | TTTA | 328 |
| Human-Exon 8 | 11 | −1 | CACTTCCTGGATGGCTTCAATGCT | TTTC | 329 |
| Human-Exon 8 | 12 | 1 | GCCTCAACAAGTGAGCATTGAAGC | TTTT | 330 |
| Human-Exon 8 | 13 | 1 | CCTCAACAAGTGAGCATTGAAGCC | TTTG | 331 |
| Human-Exon 8 | 14 | −1 | GGTGGCCTTGGCAACATTTCCACT | TTTA | 332 |
| Human-Exon 8 | 15 | −1 | GTCACTTTAGGTGGCCTTGGCAAC | TTTA | 333 |
| Human-Exon 8 | 16 | −1 | ATGATGTAACTGAAAATGTTCTTC | TTTG | 334 |
| Human-Exon 8 | 17 | −1 | CCTGTTGAGAATAGTGCATTTGAT | TTTA | 335 |
| Human-Exon 8 | 18 | 1 | CAGTTACATCATCAAATGCACTAT | TTTT | 336 |
| Human-Exon 8 | 19 | 1 | AGTTACATCATCAAATGCACTATT | TTTC | 337 |
| Human-Exon 8 | 20 | −1 | CACACTTTACCTGTTGAGAATAGT | TTTA | 338 |
| Human-Exon 8 | 21 | 1 | CTGTTTTATATGCATTTTTAGGTA | TTTT | 339 |
| Human-Exon 8 | 22 | 1 | TGTTTTATATGCATTTTTAGGTAT | TTTC | 340 |
| Human-Exon 8 | 23 | 1 | ATATGCATTTTTAGGTATTACGTG | TTTT | 341 |
| Human-Exon 8 | 24 | 1 | TATGCATTTTTAGGTATTACGTGC | TTTA | 342 |
| Human-Exon 8 | 25 | 1 | TAGGTATTACGTGCACatatatat | TTTT | 343 |
| Human-Exon 8 | 26 | 1 | AGGTATTACGTGCACatatatata | TTTT | 344 |
| Human-Exon 8 | 27 | 1 | GGTATTACGTGCACatatatatat | TTTA | 345 |
| Human-Exon 55 | 1 | −1 | AGCAACAACTATAATATTGTGCAG | TTTA | 346 |
| Human-Exon 55 | 2 | 1 | GTTCCTCCATCTTTCTCTTTTTAT | TTTA | 347 |
| Human-Exon 55 | 3 | 1 | TCTTTTTATGGAGTTCACTAGGTG | TTTC | 348 |
| Human-Exon 55 | 4 | 1 | TATGGAGTTCACTAGGTGCACCAT | TTTT | 349 |
| Human-Exon 55 | 5 | 1 | ATGGAGTTCACTAGGTGCACCATT | TTTT | 350 |
| Human-Exon 55 | 6 | 1 | TGGAGTTCACTAGGTGCACCATTC | TTTA | 351 |
| Human-Exon 55 | 7 | 1 | ATAATTGCATCTGAACATTTGGTC | TTTA | 352 |
| Human-Exon 55 | 8 | 1 | GTCCTTTGCAGGGTGAGTGAGCGA | TTTG | 353 |
| Human-Exon 55 | 9 | −1 | TTCCAAAGCAGCCTCTCGCTCACT | TTTC | 354 |
| Human-Exon 55 | 10 | 1 | CAGGGTGAGTGAGCGAGAGGCTGC | TTTG | 355 |
| Human-Exon 55 | 11 | 1 | GAAGAAACTCATAGATTACTGCAA | TTTG | 356 |
| Human-Exon 55 | 12 | −1 | CAGGTCCAGGGGGAACTGTTGCAG | TTTC | 357 |
| Human-Exon 55 | 13 | −1 | CCAGGTCCAGGGGGAACTGTTGCA | TTTT | 358 |
| Human-Exon 55 | 14 | −1 | AGCTTCTGTAAGCCAGGCAAGAAA | TTTC | 359 |
| Human-Exon 55 | 15 | 1 | TTGCCTGGCTTACAGAAGCTGAAA | TTTC | 360 |
| Human-Exon 55 | 16 | −1 | CTTACGGGTAGCATCCTGTAGGAC | TTTC | 361 |
| Human-Exon 55 | 17 | −1 | CTCCCTTGGAGTCTTCTAGGAGCC | TTTA | 362 |
| Human-Exon 55 | 18 | −1 | ACTCCCTTGGAGTCTTCTAGGAGC | TTTT | 363 |
| Human-Exon 55 | 19 | −1 | ATCAGCTCTTTTACTCCCTTGGAG | TTTC | 364 |
| Human-Exon 55 | 20 | 1 | CGCTTTAGCACTCTTGTGGATCCA | TTTC | 365 |
| Human-Exon 55 | 21 | 1 | GCACTCTTGTGGATCCAATTGAAC | TTTA | 366 |
| Human-Exon 55 | 22 | −1 | TCCCTGGCTTGTCAGTTACAAGTA | TTTG | 367 |
| Human-Exon 55 | 23 | −1 | GTCCCTGGCTTGTCAGTTACAAGT | TTTT | 368 |
| Human-Exon 55 | 24 | −1 | TTTTGTCCCTGGCTTGTCAGTTAC | TTTG | 369 |
| Human-Exon 55 | 25 | −1 | GTTTTGTCCCTGGCTTGTCAGTTA | TTTT | 370 |
| Human-Exon 55 | 26 | 1 | TACTTGTAACTGACAAGCCAGGGA | TTTG | 371 |
| Human-G1-exon51 | | 1 | gCTCCTACTCAGACTGTTACTCTG | TTTA | 372 |
| Human-G2-exon51 | | 1 | taccatgtattgctaaacaaagta | TTTC | 373 |
| Human-G3-exon51 | | −1 | attgaagagtaacaatttgagcca | TTTA | 374 |
| mouse-Exon23-G1 | | 1 | aggctctgcaaagttctTTGAAAG | TTTG | 375 |
| mouse-Exon23-G2 | | 1 | AAAGAGCAACAAAATGGCttcaac | TTTG | 376 |
| mouse-Exon23-G3 | | 1 | AAAGAGCAATAAAATGGCttcaac | TTTG | 377 |
| mouse-Exon23-G4 | | −1 | AAAGAACTTTGCAGAGCctcaaaa | TTTC | 378 |
| mouse-Exon23-G5 | | −1 | ctgaatatctatgcattaataact | TTTA | 379 |
| mouse-Exon23-G6 | | −1 | tattatattacagggcatattata | TTTC | 380 |
| mouse-Exon23-G7 | | 1 | Aggtaagccgaggtttggccttta | TTTC | 381 |
| mouse-Exon23-G8 | | 1 | cccagagtccttcaaagatattga | TTTA | 382 |

*In this table, upper case letters represent nucleotides that align to the exon sequence of the gene. Lower case letters represent nucleotides that align to the intron sequence of the gene.

TABLE E
gRNA sequences

| Targeted gRNA | Guide | | | | SEQ ID |
|---|---|---|---|---|---|
| Exon | # | Strand | gRNA sequence* | PAM | NO. |
| Human-Exon 51 | 4 | 1 | aaaaaggaaaaaagaagaaaaaga | tttt | 448 |
| Human-Exon 51 | 5 | 1 | Caaaaaggaaaaaagaagaaaaag | tttt | 449 |
| Human-Exon 51 | 6 | 1 | GCaaaaaggaaaaaagaagaaaaa | tttc | 450 |
| Human-Exon 51 | 7 | 1 | UUUUGCaaaaaggaaaaaagaaga | tttt | 451 |
| Human-Exon 51 | 8 | 1 | UUUUUGCaaaaaggaaaaaagaag | tttt | 452 |
| Human-Exon 51 | 9 | 1 | GUUUUUGCaaaaaggaaaaaagaa | tttc | 453 |
| Human-Exon 51 | 10 | 1 | AUUUUGGGUUUUUGCaaaaaggaa | tttt | 454 |
| Human-Exon 51 | 11 | 1 | UAUUUUGGGUUUUUGCaaaaagga | tttt | 455 |
| Human-Exon 51 | 12 | 1 | AUAUUUUGGGUUUUUGCaaaaagg | tttt | 456 |
| Human-Exon 51 | 13 | 1 | AAUAUUUUGGGUUUUUGCaaaaag | tttc | 457 |
| Human-Exon 51 | 14 | 1 | GCUAAAAUAUUUUGGGUUUUUGCa | tttt | 458 |
| Human-Exon 51 | 15 | 1 | AGCUAAAAUAUUUUGGGUUUUUGC | tttt | 459 |
| Human-Exon 51 | 16 | 1 | GAGCUAAAAUAUUUUGGGUUUUUG | tttG | 460 |
| Human-Exon 51 | 17 | 1 | AGAGUAACAGUCUGAGUAGGAGCU | TTTT | 461 |
| Human-Exon 51 | 18 | 1 | CAGAGUAACAGUCUGAGUAGGAGC | TTTA | 462 |
| Human-Exon 51 | 19 | −1 | GUGACACAACCUGUGGUUACUAAG | TTTC | 463 |
| Human-Exon 51 | 20 | −1 | GGUUACUAAGGAAACUGCCAUCU | TTTG | 464 |
| Human-Exon 51 | 21 | −1 | AAGGAAACUGCCAUCUCCAAACUA | TTTC | 465 |
| Human-Exon 51 | 22 | −1 | AUCAUCAAGCAGAAGGUAUGAGAA | TTTT | 466 |
| Human-Exon 51 | 23 | −1 | AGCAGAAGGUAUGAGAAAAAAUGA | TTTA | 467 |
| Human-Exon 51 | 24 | −1 | GCAGAAGGUAUGAGAAAAAAUGAU | TTTT | 468 |
| Human-Exon 51 | 25 | −1 | UAAAAGUUGGCAGAAGUUUUUCUU | TTTA | 469 |
| Human-Exon 51 | 26 | −1 | AAAAGUUGGCAGAAGUUUUUCUUU | TTTT | 470 |
| Human-Exon 51 | 27 | 1 | GGUGGAAAAUCUUCAUUUUAAAGA | TTTT | 471 |
| Human-Exon 51 | 28 | 1 | UGGUGGAAAAUCUUCAUUUUAAAG | TTTT | 472 |
| Human-Exon 51 | 29 | 1 | UUGGUGGAAAAUCUUCAUUUUAAA | TTTC | 473 |
| Human-Exon 51 | 30 | 1 | GUGAUUGGUGGAAAAUCUUCAUUU | TTTA | 474 |
| Human-Exon 51 | 31 | 1 | CUAGGAGAGUAAAGUGAUUGGUGG | TTTT | 475 |
| Human-Exon 51 | 32 | 1 | UCUAGGAGAGUAAAGUGAUUGGUG | TTTC | 476 |
| Human-Exon 51 | 33 | 1 | CUGGUGGGAAAUGGUCUAGGAGA | TTTA | 477 |
| Human-Exon 45 | 1 | −1 | guagcacacuguuuaaucuuuucu | tttg | 478 |
| Human-Exon 45 | 2 | −1 | cacacuguuuaaucuuuucucaaa | TTTa | 479 |
| Human-Exon 45 | 3 | −1 | acacuguuuaaucuuuucucaaau | TTTT | 480 |
| Human-Exon 45 | 4 | −1 | cacuguuuaaucuuuucucaaauA | TTTT | 481 |
| Human-Exon 45 | 5 | 1 | AUGUCUUUUUauuugagaaaagau | ttta | 482 |
| Human-Exon 45 | 6 | 1 | AAGCCCCAUGUCUUUUUauuugag | tttt | 483 |
| Human-Exon 45 | 7 | 1 | GAAGCCCCAUGUCUUUUUauuuga | tttc | 484 |
| Human-Exon 45 | 8 | 1 | GUAAGAUACCAAAAAGGCAAAACA | TTTT | 485 |
| Human-Exon 45 | 9 | 1 | UGUAAGAUACCAAAAAGGCAAAAC | TTTT | 486 |
| Human-Exon 45 | 10 | 1 | CUGUAAGAUACCAAAAAGGCAAAA | TTTG | 487 |
| Human-Exon 45 | 11 | 1 | GUUCCUGUAAGAUACCAAAAAGGC | TTTT | 488 |
| Human-Exon 45 | 12 | 1 | AGUUCCUGUAAGAUACCAAAAAGG | TTTG | 489 |
| Human-Exon 45 | 13 | 1 | UCCUGGAGUUCCUGUAAGAUACCA | TTTT | 490 |
| Human-Exon 45 | 14 | 1 | AUCCUGGAGUUCCUGUAAGAUACC | TTTT | 491 |
| Human-Exon 45 | 15 | −1 | GGGAAGAAAUAAUUCAGCAAUCCU | TTTG | 492 |
| Human-Exon 45 | 16 | −1 | GGAAGAAAUAAUUCAGCAAUCCUC | TTTT | 493 |
| Human-Exon 45 | 17 | −1 | GAAGAAAUAAUUCAGCAAUCCUCA | TTTT | 494 |
| Human-Exon 45 | 18 | −1 | AAAACAGAUGCCAGUAUUCUACAG | TTTC | 495 |
| Human-Exon 45 | 19 | −1 | AAACAGAUGCCAGUAUUCUACAGG | TTTT | 496 |
| Human-Exon 45 | 20 | −1 | AACAGAUGCCAGUAUUCUACAGGA | TTTT | 497 |
| Human-Exon 45 | 21 | −1 | GAAUCUGCGGUGGCAGGAGGUCUG | TTTG | 498 |
| Human-Exon 45 | 22 | −1 | AGGUCUGCAAACAGCUGUCAGACA | TTTC | 499 |
| Human-Exon 45 | 23 | −1 | GGUCUGCAAACAGCUGUCAGACAG | TTTT | 500 |
| Human-Exon 45 | 24 | −1 | GUCUGCAAACAGCUGUCAGACAGA | TTTT | 501 |
| Human-Exon 45 | 25 | −1 | UCUGCAAACAGCUGUCAGACAGAA | TTTT | 502 |
| Human-Exon 45 | 26 | −1 | UAGGGCGACAGAUCUAAUAGGAAU | TTTC | 503 |
| Human-Exon 45 | 27 | −1 | AGGGCGACAGAUCUAAUAGGAAUG | TTTT | 504 |
| Human-Exon 45 | 28 | 1 | UAAAGAAAGCUUAAAAAGUCUGCU | TTTT | 505 |
| Human-Exon 45 | 29 | 1 | CUAAAGAAAGCUUAAAAAGUCUGC | TTTA | 506 |
| Human-Exon 45 | 30 | 1 | AAAUAUUCUUCUAAAGAAAGCUUA | TTTT | 507 |
| Human-Exon 45 | 31 | 1 | GAAAUAUUCUUCUAAAGAAAGCUU | TTTT | 508 |
| Human-Exon 45 | 32 | 1 | UGAAAUAUUCUUCUAAAGAAAGCU | TTTA | 509 |
| Human-Exon 45 | 33 | 1 | UCUCUCAUGAAAUAUUCUUCUAAA | TTTC | 510 |
| Human-Exon 45 | 34 | 1 | AUAAUCUCUCAUGAAAUAUUCUUC | TTTA | 511 |
| Human-Exon 44 | 1 | 1 | GCGUAUAUUUUUUGGUUAUACUGA | TTTG | 512 |
| Human-Exon 44 | 2 | 1 | ucaagaaaaauagauggauuaugu | tttt | 513 |
| Human-Exon 44 | 3 | 1 | aucaagaaaaauagauggauuaug | ttta | 514 |
| Human-Exon 44 | 4 | 1 | CAGGUaaaagcauauggaucaaga | tttt | 515 |
| Human-Exon 44 | 5 | 1 | GCAGGUaaaagcauauggaucaag | tttt | 516 |
| Human-Exon 44 | 6 | 1 | UGCAGGUaaaagcauauggaucaa | tttc | 517 |
| Human-Exon 44 | 7 | −1 | CAGGCGAUUUGACAGAUCUGUUGA | TTTC | 518 |
| Human-Exon 44 | 8 | 1 | AGAUCUGUCAAAUCGCCUGCAGGU | tttt | 519 |
| Human-Exon 44 | 9 | 1 | CAGAUCUGUCAAAUCGCCUGCAGG | tttA | 520 |
| Human-Exon 44 | 10 | 1 | GCCGCCAUUUCUCAACAGAUCUGU | TTTG | 521 |
| Human-Exon 44 | 11 | −1 | AAUGGCGGCGUUUUCAUUAUGAUA | TTTA | 522 |
| Human-Exon 44 | 12 | 1 | AUUAAAUAUCUUUAUAUCAUAAUG | TTTT | 523 |
| Human-Exon 44 | 13 | −1 | UGAGAAUUGGGAACAUGCUAAAUA | TTTG | 524 |
| Human-Exon 44 | 14 | −1 | GGUAAGUCUUUGAUUUGUUUUUUC | TTTC | 525 |
| Human-Exon 44 | 15 | 1 | AAAUACAAUUUCGAAAAAACAAAU | TTTG | 526 |
| Human-Exon 44 | 16 | 1 | AAGAUAAAUACAAUUUCGAAAAAA | TTTG | 527 |
| Human-Exon 44 | 17 | 1 | GCUGAAGAUAAAUACAAUUUCGAA | TTTT | 528 |
| Human-Exon 44 | 18 | 1 | UGCUGAAGAUAAAUACAAUUUCGA | TTTT | 529 |
| Human-Exon 44 | 19 | 1 | GUGCUGAAGAUAAAUACAAUUUCG | TTTT | 530 |
| Human-Exon 44 | 20 | 1 | UGUGCUGAAGAUAAAUACAAUUUC | TTTC | 531 |
| Human-Exon 44 | 21 | −1 | GCACAUCUGGACUCUUUAACUUCU | TTTA | 532 |
| Human-Exon 44 | 22 | 1 | UAAAGAGUCCAGAUGUGCUGAAGA | TTTA | 533 |
| Human-Exon 44 | 23 | −1 | AAGAUCAGGUUCUGAAGGGUGAUG | TTTC | 534 |
| Human-Exon 44 | 24 | 1 | UUCAGAACCUGAUCUUUAAGAAGU | TTTA | 535 |
| Human-Exon 44 | 25 | 1 | AAUAUAAUGAUGACAACAACAGUC | TTTT | 536 |
| Human-Exon 44 | 26 | 1 | UAAUAUAAUGAUGACAACAACAGU | TTTG | 537 |
| Human-Exon 53 | 1 | −1 | UUUAUUUUUCCUUUUAUUCUAGUU | TTTC | 538 |
| Human-Exon 53 | 2 | 1 | AAAGGAAAAAUAAAUAUAUAGUAG | TTTA | 539 |
| Human-Exon 53 | 3 | 1 | UUUCAACUAGAAUAAAAGGAAAAA | TTTA | 540 |
| Human-Exon 53 | 4 | 1 | AUUCUUUCAACUAGAAUAAAAGGA | TTTT | 541 |
| Human-Exon 53 | 5 | 1 | AAUUCUUUCAACUAGAAUAAAAGG | TTTT | 542 |
| Human-Exon 53 | 6 | 1 | GAAUUCUUUCAACUAGAAUAAAAG | TTTC | 543 |
| Human-Exon 53 | 7 | 1 | AUUCUGAAUUCUUUCAACUAGAAU | TTTT | 544 |
| Human-Exon 53 | 8 | 1 | GAUUCUGAAUUCUUUCAACUAGAA | TTTA | 545 |
| Human-Exon 53 | 9 | −1 | CAGAACCGGAGGCAACAGUUGAAU | TTTC | 546 |
| Human-Exon 53 | 10 | −1 | GGAGGCAACAGUUGAAUGAAAUGU | TTTA | 547 |
| Human-Exon 53 | 11 | −1 | UAUACAGUAGAUGCAAUCCAAAAG | TTTT | 548 |
| Human-Exon 53 | 12 | −1 | GAUGCAAUCCAAAAGAAAAUCACA | TTTC | 549 |
| Human-Exon 53 | 13 | −1 | AAUCACAGAAACCAAGGUUAGUAU | TTTG | 550 |
| Human-Exon 53 | 14 | −1 | AGGUUAGUAUCAAAGAUACCUUU | TTTA | 551 |
| Human-Exon 53 | 15 | −1 | GGUUAGUAUCAAAGAUACCUUUUU | TTTT | 552 |
| Human-Exon 53 | 16 | −1 | AGUAUCAAAGAUACCUUUUUAAAA | TTTA | 553 |
| Human-Exon 53 | 17 | −1 | GUAUCAAAGAUACCUUUUUAAAAU | TTTT | 554 |
| Human-Exon 46 | 1 | −1 | UGUUUGUGUCCCAGUUUGCAUUAA | TTTG | 555 |
| Human-Exon 46 | 2 | 1 | CUGGGACACAAACAUGGCAAUUUA | TTTT | 556 |
| Human-Exon 46 | 3 | 1 | ACUGGGACACAAACAUGGCAAUUU | TTTT | 557 |
| Human-Exon 46 | 4 | 1 | AACUGGGACACAAACAUGGCAAUU | TTTA | 558 |
| Human-Exon 46 | 5 | 1 | UAUUUGUUAAUGCAAACUGGGACA | TTTG | 559 |
| Human-Exon 46 | 6 | −1 | ACAAAUAGUUUGAGAACUAUGUUG | tttC | 560 |
| Human-Exon 46 | 7 | −1 | CAAAUAGUUUGAGAACUAUGUUGG | tttt | 561 |
| Human-Exon 46 | 8 | −1 | AAAUAGUUUGAGAACUAUGUUGGa | tttt | 562 |
| Human-Exon 46 | 9 | −1 | AUAGUUUGAGAACUAUGUUGGaaa | tttt | 563 |
| Human-Exon 46 | 10 | −1 | UAGUUUGAGAACUAUGUUGGaaaa | tttt | 564 |
| Human-Exon 46 | 11 | −1 | AGUUUGAGAACUAUGUUGGaaaaa | tttt | 565 |
| Human-Exon 46 | 12 | 1 | UAGUUCUCAAACUAUUUGUUAAUG | TTTG | 566 |
| Human-Exon 46 | 13 | 1 | UAuuuuuuuuuCCAACAUAGUUCU | TTTG | 567 |
| Human-Exon 46 | 14 | −1 | CUUCUUUCUCCAGGCUAGAAGAAC | TTTT | 568 |
| Human-Exon 46 | 15 | 1 | CUUCUAGCCUGGAGAAAGAAGAAU | TTTT | 569 |
| Human-Exon 46 | 16 | 1 | UCUUCUAGCCUGGAGAAAGAAGAA | TTTA | 570 |
| Human-Exon 46 | 17 | 1 | AUUCUUUUGUUCUUCUAGCCUGGA | TTTC | 571 |
| Human-Exon 46 | 18 | −1 | CAAAAGAAUAUCUUGUCAGAAUUU | TTTG | 572 |
| Human-Exon 46 | 19 | −1 | CUGGAAAAGAGCAGCAACUAAAAG | TTTT | 573 |
| Human-Exon 46 | 20 | −1 | CAAGUCAAGGUAAUUUUAUUUUCU | TTTG | 574 |
| Human-Exon 46 | 21 | −1 | CAAAUCCCCCAGGGCCUGCUUGCA | TTTA | 575 |
| Human-Exon 46 | 22 | 1 | AGGCCCUGGGGGAUUUGAGAAAAU | TTTT | 576 |
| Human-Exon 46 | 23 | 1 | CAGGCCCUGGGGGAUUUGAGAAAA | TTTA | 577 |
| Human-Exon 46 | 24 | 1 | CAAGCAGGCCCUGGGGGAUUUGAG | TTTT | 578 |
| Human-Exon 46 | 25 | 1 | GCAAGCAGGCCCUGGGGGAUUUGA | TTTC | 579 |
| Human-Exon 46 | 26 | 1 | GCAGAAAACCAAUGAUUGAAUUAA | TTTT | 580 |
| Human-Exon 46 | 27 | 1 | GGCAGAAAACCAAUGAUUGAAUUA | TTTT | 581 |
| Human-Exon 46 | 28 | 1 | GGGCAGAAAACCAAUGAUUGAAUU | TTTT | 582 |
| Human-Exon 46 | 29 | 1 | UGGGCAGAAAACCAAUGAUUGAAU | TTTA | 583 |
| Human-Exon 46 | 30 | −1 | AUUAGGUUAUUCAUAGUUCCUUGC | TTTA | 584 |
| Human-Exon 46 | 31 | 1 | AACUAUGAAUAACCUAAUGGGCAG | TTTT | 585 |
| Human-Exon 46 | 32 | 1 | GAACUAUGAAUAACCUAAUGGGCA | TTTC | 586 |
| Human-Exon 52 | 1 | −1 | UAUUUCCUGUUAAAUUGUUUUCUA | TTTA | 587 |
| Human-Exon 52 | 2 | 1 | GGUUUAUAGAAAACAAUUUAACAG | TTTC | 588 |
| Human-Exon 52 | 3 | −1 | AUACAGUAACAUCUUUUUUAUUUC | TTTA | 589 |
| Human-Exon 52 | 4 | −1 | UACAGUAACAUCUUUUUUAUUUCU | TTTT | 590 |
| Human-Exon 52 | 5 | 1 | AUGUUACUGUAUAAGGGUUUAUAG | TTTT | 591 |
| Human-Exon 52 | 6 | 1 | GAUGUUACUGUAUAAGGGUUUAUA | TTTC | 592 |
| Human-Exon 52 | 7 | 1 | CAGCCAAAACACUUUUAGAAAUAA | TTTT | 593 |
| Human-Exon 52 | 8 | 1 | CCAGCCAAAACACUUUUAGAAAUA | TTTT | 594 |
| Human-Exon 52 | 9 | 1 | ACCAGCCAAAACACUUUUAGAAAU | TTTT | 595 |
| Human-Exon 52 | 10 | 1 | GACCAGCCAAAACACUUUUAGAAA | TTTA | 596 |
| Human-Exon 52 | 11 | 1 | GUGAGACCAGCCAAAACACUUUUA | TTTC | 597 |
| Human-Exon 52 | 12 | −1 | AAUUGUACUUUACUUUGUAUUAUG | TTTA | 598 |
| Human-Exon 52 | 13 | −1 | AUUGUACUUUACUUUGUAUUAUGU | TTTT | 599 |
| Human-Exon 52 | 14 | 1 | UAAAGUACAAUUGUGAGACCAGCC | TTTT | 600 |
| Human-Exon 52 | 15 | 1 | GUAAAGUACAAUUGUGAGACCAGC | TTTG | 601 |
| Human-Exon 52 | 16 | 1 | GUAUUCCUUUUACAUAAUACAAAG | TTTA | 602 |
| Human-Exon 52 | 17 | 1 | GUUGUGUAUUCCUUUUACAUAAUA | TTTG | 603 |
| Human-Exon 52 | 18 | 1 | AUCCUGCAUUGUUGCCUGUAAGAA | TTTG | 604 |
| Human-Exon 52 | 19 | 1 | UUCCAACUGGGGACGCCUCUGUUC | TTTG | 605 |
| Human-Exon 52 | 20 | −1 | UUGGAAGAACUCAUUACCGCUGCC | TTTG | 606 |
| Human-Exon 52 | 21 | −1 | UCAUUACCGCUGCCCAAAAUUUGA | TTTT | 607 |
| Human-Exon 52 | 22 | 1 | CUCUUGAUUGCUGGUCUUGUUUUU | TTTG | 608 |
| Human-Exon 52 | 23 | −1 | GUUUUUUAACAAGCAUGGGACACA | TTTG | 609 |
| Human-Exon 52 | 24 | 1 | CUUUGUGUGUCCCAUGCUUGUUAA | TTTT | 610 |
| Human-Exon 52 | 25 | 1 | GCUUUGUGUGUCCCAUGCUUGUUA | TTTT | 611 |
| Human-Exon 52 | 26 | 1 | UGCUUUGUGUGUCCCAUGCUUGUU | TTTT | 612 |
| Human-Exon 52 | 27 | 1 | UUGCUUUGUGUGUCCCAUGCUUGU | TTTA | 613 |
| Human-Exon 52 | 28 | −1 | AGCAAGAUGCAUGACAAGUUUCAA | TTTA | 614 |
| Human-Exon 52 | 29 | −1 | GCAAGAUGCAUGACAAGUUUCAAU | TTTT | 615 |
| Human-Exon 52 | 30 | −1 | CAAGAUGCAUGACAAGUUUCAAUA | TTTT | 616 |
| Human-Exon 52 | 31 | 1 | GAUAUAUGAACUUAAGUUUUUAUU | TTTC | 617 |
| Human-Exon 50 | 1 | −1 | AUAGAAAUCCAAUAAUAUAUUCAC | TTTG | 618 |
| Human-Exon 50 | 2 | −1 | AUUAAGAUGUUCAUGAAUUAUCUU | TTTG | 619 |
| Human-Exon 50 | 3 | −1 | UAAGUAAUGUGUAUGCUUUUCUGU | TTTA | 620 |
| Human-Exon 50 | 4 | 1 | AUCUUCUAACUUCCUCUUUAACAG | TTTT | 621 |
| Human-Exon 50 | 5 | 1 | GAUCUUCUAACUUCCUCUUUAACA | TTTC | 622 |
| Human-Exon 50 | 6 | −1 | AUCUGAGCUCUGAGUGGAAGGCGG | TTTA | 623 |
| Human-Exon 50 | 7 | −1 | ACCGUUUACUUCAAGAGCUGAGGG | TTTG | 624 |
| Human-Exon 50 | 8 | 1 | CUGCUUUGCCCUCAGCUCUUGAAG | TTTA | 625 |
| Human-Exon 50 | 9 | −1 | UCUCUUUGGCUCUAGCUAUUUGUU | TTTG | 626 |
| Human-Exon 50 | 10 | −1 | CUCUUUGGCUCUAGCUAUUUGUUC | TTTT | 627 |
| Human-Exon 50 | 11 | 1 | CACUUUUGAACAAAUAGCUAGAGC | TTTG | 628 |
| Human-Exon 50 | 12 | 1 | UCACUUCAUAGUUGCACUUUUGAA | TTTG | 629 |
| Human-Exon 50 | 13 | −1 | AUGAAGUGAUGACUGGGUGAGAGA | TTTC | 630 |
| Human-Exon 50 | 14 | −1 | UGAAGUGAUGACUGGGUGAGAGAG | TTTT | 631 |
| Human-Exon 43 | 1 | 1 | AAGAGAAAAauauauauauauaua | TTTG | 632 |
| Human-Exon 43 | 2 | 1 | GAAUUAGCUGUCUAUAGAAAGAGA | tTTT | 633 |
| Human-Exon 43 | 3 | 1 | UGAAUUAGCUGUCUAUAGAAAGAG | TTTT | 634 |
| Human-Exon 43 | 4 | −1 | AGCUAAUUCAUUUUUUUACUGUUU | TTTA | 635 |
| Human-Exon 43 | 5 | 1 | AUGAAUUAGCUGUCUAUAGAAAGA | TTTC | 636 |
| Human-Exon 43 | 6 | −1 | GCUAAUUCAUUUUUUUACUGUUUU | TTTT | 637 |
| Human-Exon 43 | 7 | 1 | AAAAAAAUGAAUUAGCUGUCUAUA | TTTC | 638 |
| Human-Exon 43 | 8 | −1 | UUAAAAUUUUUAUAUUACAGAAUA | TTTA | 639 |
| Human-Exon 43 | 9 | −1 | UAAAAUUUUUAUAUUACAGAAUAU | TTTT | 640 |
| Human-Exon 43 | 10 | 1 | AUAUAAAAAUUUUAAAACAGUAAA | TTTT | 641 |
| Human-Exon 43 | 11 | 1 | AAUAUAAAAAUUUUAAAACAGUAA | TTTT | 642 |
| Human-Exon 43 | 12 | 1 | UAAUAUAAAAAUUUUAAAACAGUA | TTTT | 643 |
| Human-Exon 43 | 13 | 1 | GUAAUAUAAAAAUUUUAAAACAGU | TTTT | 644 |
| Human-Exon 43 | 14 | 1 | UGUAAUAUAAAAAUUUUAAAACAG | TTTA | 645 |
| Human-Exon 43 | 15 | 1 | UAUAUUCUGUAAUAUAAAAAUUUU | TTTT | 646 |
| Human-Exon 43 | 16 | 1 | UUAUAUUCUGUAAUAUAAAAAUUU | TTTA | 647 |
| Human-Exon 43 | 17 | −1 | CAGAAUAUAAAAGAUAGUCUACAA | TTTG | 648 |
| Human-Exon 43 | 18 | 1 | CUAUCUUUUAUAUUCUGUAAUAUA | TTTT | 649 |
| Human-Exon 43 | 19 | 1 | ACUAUCUUUUAUAUUCUGUAAUAU | TTTT | 650 |
| Human-Exon 43 | 20 | 1 | GACUAUCUUUUAUAUUCUGUAAUA | TTTA | 651 |
| Human-Exon 43 | 21 | −1 | CAUAGCAAGAAGACAGCAGCAUUG | TTTG | 652 |
| Human-Exon 43 | 22 | 1 | CAUUUUGUUAACUUUUUCCCAUUG | TTTC | 653 |
| Human-Exon 43 | 23 | −1 | CAUAUAUUUUUCUUGAUACUUGCA | TTTC | 654 |
| Human-Exon 43 | 24 | 1 | AAAUCAUUUCUGCAAGUAUCAAGA | TTTT | 655 |
| Human-Exon 43 | 25 | 1 | CAAAUCAUUUCUGCAAGUAUCAAG | TTTT | 656 |
| Human-Exon 43 | 26 | 1 | ACAAAUCAUUUCUGCAAGUAUCAA | TTTC | 657 |
| Human-Exon 43 | 27 | 1 | AUAAAUUCUACAGUUCCCUGAAAA | TTTG | 658 |
| Human-Exon 43 | 28 | −1 | GAAUUUAUUUCAGUACCCUCCAUG | TTTC | 659 |
| Human-Exon 43 | 29 | −1 | AAUUUAUUUCAGUACCCUCCAUGG | TTTT | 660 |
| Human-Exon 43 | 30 | 1 | UGAAAUAAAUUCUACAGUUCCCUG | TTTT | 661 |
| Human-Exon 43 | 31 | −1 | AUUUAUUUCAGUACCCUCCAUGGA | TTTT | 662 |
| Human-Exon 43 | 32 | 1 | CUGAAAUAAAUUCUACAGUUCCCU | TTTC | 663 |
| Human-Exon 43 | 33 | −1 | UUUAUUUCAGUACCCUCCAUGGAA | TTTT | 664 |
| Human-Exon 43 | 34 | −1 | UACCCUCCAUGGAAAAAAGACAGG | TTTC | 665 |
| Human-Exon 43 | 35 | −1 | ACCCUCCAUGGAAAAAAGACAGGG | TTTT | 666 |
| Human-Exon 43 | 36 | −1 | CCCUCCAUGGAAAAAAGACAGGGA | TTTT | 667 |
| Human-Exon 43 | 37 | 1 | UUUUUUCCAUGGAGGGUACUGAAA | TTTA | 668 |
| Human-Exon 43 | 38 | 1 | UGUCUUUUUUCCAUGGAGGGUACU | TTTC | 669 |
| Human-Exon 6 | 1 | 1 | CCUUGAGCAAGAACCAUGCAAACU | TTTA | 670 |
| Human-Exon 6 | 2 | −1 | UGCUCAAGGAAUGCAUUUUCUUAU | TTTC | 671 |
| Human-Exon 6 | 3 | −1 | GCUCAAGGAAUGCAUUUUCUUAUG | TTTT | 672 |
| Human-Exon 6 | 4 | 1 | UGCAUUCCUUGAGCAAGAACCAUG | TTTG | 673 |
| Human-Exon 6 | 5 | −1 | GAAAAUUUAUUUCCACAUGUAGGU | TTTG | 674 |
| Human-Exon 6 | 6 | −1 | AAAAUUUAUUUCCACAUGUAGGUC | TTTT | 675 |
| Human-Exon 6 | 7 | −1 | AAAUUUAUUUCCACAUGUAGGUCA | TTTT | 676 |
| Human-Exon 6 | 8 | 1 | CAUGUGGAAAUAAAUUUUCAUAAG | TTTT | 677 |
| Human-Exon 6 | 9 | 1 | ACAUGUGGAAAUAAAUUUUCAUAA | TTTC | 678 |
| Human-Exon 6 | 10 | −1 | CCACAUGUAGGUCAAAAAUGUAAU | TTTC | 679 |
| Human-Exon 6 | 11 | −1 | CACAUGUAGGUCAAAAAUGUAAUG | TTTT | 680 |
| Human-Exon 6 | 12 | −1 | ACAUGUAGGUCAAAAAUGUAAUGA | TTTT | 681 |
| Human-Exon 6 | 13 | 1 | ACAUUUUUGACCUACAUGUGGAAA | TTTA | 682 |
| Human-Exon 6 | 14 | 1 | CAUUACAUUUUUGACCUACAUGUG | TTTC | 683 |
| Human-Exon 6 | 15 | −1 | AAAAAUAUCAUGGCUGGAUUGCAA | TTTG | 684 |
| Human-Exon 6 | 16 | −1 | GCUGGAUUGCAACAAACCAACAGU | TTTC | 685 |
| Human-Exon 6 | 17 | −1 | CUGGAUUGCAACAAACCAACAGUG | TTTT | 686 |
| Human-Exon 6 | 18 | 1 | CCUAUGACUAUGGAUGAGAGCAUU | TTTG | 687 |
| Human-Exon 6 | 19 | −1 | UAGGUAAGAAGAUUACUGAGACAU | TTTA | 688 |
| Human-Exon 6 | 20 | −1 | AUUACUGAGACAUUAAAUAACUUG | TTTA | 689 |
| Human-Exon 6 | 21 | −1 | UUACUGAGACAUUAAAUAACUUGU | TTTT | 690 |
| Human-Exon 6 | 22 | 1 | GGGGAAAAAUAUGUCAUCAGAGUC | TTTA | 691 |
| Human-Exon 6 | 23 | 1 | CAUGAUCUGGAACCAUACUGGGGA | TTTT | 692 |
| Human-Exon 6 | 24 | 1 | ACAUGAUCUGGAACCAUACUGGGG | TTTT | 693 |
| Human-Exon 6 | 25 | 1 | GACAUGAUCUGGAACCAUACUGGG | TTTC | 694 |
| Human-Exon 7 | 1 | 1 | uacacacauacacaAAGACAAAUA | TTTA | 695 |
| Human-Exon 7 | 2 | 1 | uacacauacacacauacacaAAGA | TTTG | 696 |
| Human-Exon 7 | 3 | 1 | aacacauacacauacacacauaca | TTtg | 697 |
| Human-Exon 7 | 4 | 1 | AUUCCAGUCAAAUAGGUCUGGCCU | ttTT | 698 |
| Human-Exon 7 | 5 | 1 | UAUUCCAGUCAAAUAGGUCUGGCC | tTTA | 699 |
| Human-Exon 7 | 6 | 1 | GCUGGCAAACCACACUAUUCCAGU | TTTG | 700 |
| Human-Exon 7 | 7 | 1 | AGUCGUUGUGUGGCUGACUGCUGG | TTTG | 701 |
| Human-Exon 7 | 8 | −1 | CGCCAGAUAUCAAUUAGGCAUAGA | TTTC | 702 |
| Human-Exon 7 | 9 | −1 | AAACUACUCGAUCCUGAAGGUUGG | TTTA | 703 |
| Human-Exon 7 | 10 | 1 | CAUACUAAAAGCAGUGGUAGUCCA | TTTC | 704 |
| Human-Exon 7 | 11 | 1 | GAAAACAUUAAACUCUACCAUACU | TTTT | 705 |
| Human-Exon 7 | 12 | 1 | UGAAAACAUUAAACUCUACCAUAC | TTTA | 706 |
| Human-Exon 8 | 1 | −1 | UUGUUCAUUAUCCUUUUAGAGUCU | TTTG | 707 |
| Human-Exon 8 | 2 | 1 | AAAGGAUAAUGAACAAAUCAAAGU | TTTA | 708 |
| Human-Exon 8 | 3 | −1 | UAUCCUUUUAGAGUCUCAAAUAUA | TTTC | 709 |
| Human-Exon 8 | 4 | 1 | ACUCUAAAAGGAUAAUGAACAAAU | TTTG | 710 |
| Human-Exon 8 | 5 | −1 | UUUUAGAGUCUCAAAUAUAGAAAC | TTTG | 711 |
| Human-Exon 8 | 6 | −1 | UUUAGAGUCUCAAAUAUAGAAACC | TTTT | 712 |
| Human-Exon 8 | 7 | −1 | UUAGAGUCUCAAAUAUAGAAACCA | TTTT | 713 |
| Human-Exon 8 | 8 | 1 | UUGAGACUCUAAAAGGAUAAUGAA | TTTG | 714 |
| Human-Exon 8 | 9 | 1 | UUUGGUUUCUAUAUUUGAGACUCU | TTTT | 715 |
| Human-Exon 8 | 10 | 1 | UUUUGGUUUCUAUAUUUGAGACUC | TTTA | 716 |
| Human-Exon 8 | 11 | −1 | AGCAUUGAAGCCAUCCAGGAAGUG | TTTC | 717 |
| Human-Exon 8 | 12 | 1 | GCUUCAAUGCUCACUUGUUGAGGC | TTTT | 718 |
| Human-Exon 8 | 13 | 1 | GGCUUCAAUGCUCACUUGUUGAGG | TTTG | 719 |
| Human-Exon 8 | 14 | −1 | AGUGGAAAUGUUGCCAAGGCCACC | TTTA | 720 |
| Human-Exon 8 | 15 | −1 | GUUGCCAAGGCCACCUAAAGUGAC | TTTA | 721 |
| Human-Exon 8 | 16 | −1 | GAAGAACAUUUUCAGUUACAUCAU | TTTG | 722 |
| Human-Exon 8 | 17 | −1 | AUCAAAUGCACUAUUCUCAACAGG | TTTA | 723 |
| Human-Exon 8 | 18 | 1 | AUAGUGCAUUUGAUGAUGUAACUG | TTTT | 724 |
| Human-Exon 8 | 19 | 1 | AAUAGUGCAUUUGAUGAUGUAACU | TTTC | 725 |
| Human-Exon 8 | 20 | −1 | ACUAUUCUCAACAGGUAAAGUGUG | TTTA | 726 |
| Human-Exon 8 | 21 | 1 | UACCUAAAAAUGCAUAUAAAACAG | TTTT | 727 |
| Human-Exon 8 | 22 | 1 | AUACCUAAAAAUGCAUAUAAAACA | TTTC | 728 |
| Human-Exon 8 | 23 | 1 | CACGUAAUACCUAAAAAUGCAUAU | TTTT | 729 |
| Human-Exon 8 | 24 | 1 | GCACGUAAUACCUAAAAAUGCAUA | TTTA | 730 |
| Human-Exon 8 | 25 | 1 | auauauauGUGCACGUAAUACCUA | TTTT | 731 |
| Human-Exon 8 | 26 | 1 | uauauauauGUGCACGUAAUACCU | TTTT | 732 |
| Human-Exon 8 | 27 | 1 | auauauauauGUGCACGUAAUACC | TTTA | 733 |
| Human-Exon 55 | 1 | −1 | CUGCACAAUAUUAUAGUUGUUGCU | TTTA | 734 |
| Human-Exon 55 | 2 | 1 | AUAAAAAGAGAAAGAUGGAGGAAC | TTTA | 735 |
| Human-Exon 55 | 3 | 1 | CACCUAGUGAACUCCAUAAAAAGA | TTTC | 736 |
| Human-Exon 55 | 4 | 1 | AUGGUGCACCUAGUGAACUCCAUA | TTTT | 737 |
| Human-Exon 55 | 5 | 1 | AAUGGUGCACCUAGUGAACUCCAU | TTTT | 738 |
| Human-Exon 55 | 6 | 1 | GAAUGGUGCACCUAGUGAACUCCA | TTTA | 739 |
| Human-Exon 55 | 7 | 1 | GACCAAAUGUUCAGAUGCAAUUAU | TTTA | 740 |
| Human-Exon 55 | 8 | 1 | UCGCUCACUCACCCUGCAAAGGAC | TTTG | 741 |
| Human-Exon 55 | 9 | −1 | AGUGAGCGAGAGGCUGCUUUGGAA | TTTC | 742 |
| Human-Exon 55 | 10 | 1 | GCAGCCUCUCGCUCACUCACCCUG | TTTG | 743 |
| Human-Exon 55 | 11 | 1 | UUGCAGUAAUCUAUGAGUUUCUUC | TTTG | 744 |
| Human-Exon 55 | 12 | −1 | CUGCAACAGUUCCCCCUGGACCUG | TTTC | 745 |
| Human-Exon 55 | 13 | −1 | UGCAACAGUUCCCCCUGGACCUGG | TTTT | 746 |
| Human-Exon 55 | 14 | −1 | UUUCUUGCCUGGCUUACAGAAGCU | TTTC | 747 |
| Human-Exon 55 | 15 | 1 | UUUCAGCUUCUGUAAGCCAGGCAA | TTTC | 748 |
| Human-Exon 55 | 16 | −1 | GUCCUACAGGAUGCUACCCGUAAG | TTTC | 749 |
| Human-Exon 55 | 17 | −1 | GGCUCCUAGAAGACUCCAAGGGAG | TTTA | 750 |
| Human-Exon 55 | 18 | −1 | GCUCCUAGAAGACUCCAAGGGAGU | TTTT | 751 |
| Human-Exon 55 | 19 | −1 | CUCCAAGGGAGUAAAAGAGCUGAU | TTTC | 752 |
| Human-Exon 55 | 20 | 1 | UGGAUCCACAAGAGUGCUAAAGCG | TTTC | 753 |
| Human-Exon 55 | 21 | 1 | GUUCAAUUGGAUCCACAAGAGUGC | TTTA | 754 |
| Human-Exon 55 | 22 | −1 | UACUUGUAACUGACAAGCCAGGGA | TTTG | 755 |
| Human-Exon 55 | 23 | −1 | ACUUGUAACUGACAAGCCAGGGAC | TTTT | 756 |
| Human-Exon 55 | 24 | −1 | GUAACUGACAAGCCAGGGACAAAA | TTTG | 757 |
| Human-Exon 55 | 25 | −1 | UAACUGACAAGCCAGGGACAAAAC | TTTT | 758 |
| Human-Exon 55 | 26 | 1 | UCCCUGGCUUGUCAGUUACAAGUA | TTTG | 759 |
| Human-G1-exon51 | | 1 | CAGAGUAACAGUCUGAGUAGGAGc | TTTA | 760 |
| Human-G2-exon51 | | 1 | uacuuuguuuagcaauacauggua | TTTC | 761 |
| Human-G3-exon51 | | −1 | uggcucaaauuguuacucuucaau | TTTA | 762 |
| mouse-Exon23-G1 | | 1 | CUUUCAAagaacuuugcagagccu | TTTG | 763 |
| mouse-Exon23-G2 | | 1 | guugaaGCCAUUUUGUUGCUCUUU | TTTG | 764 |
| mouse-Exon23-G3 | | 1 | guugaaGCCAUUUUAUUGCUCUUU | TTTG | 765 |
| mouse-Exon23-G4 | | −1 | uuuugagGCUCUGCAAAGUUCUUU | TTTC | 766 |
| mouse-Exon23-G5 | | −1 | aguuauuaaugcauagauauucag | TTTA | 767 |
| mouse-Exon23-G6 | | −1 | uauaauaugcccuguaauauaaua | TTTC | 768 |
| mouse-Exon23-G7 | | 1 | uaaaggccaaaccucggcuuaccU | TTTC | 769 |
| mouse-Exon23-G8 | | 1 | ucaauaucuuugaaggacucuggg | TTTA | 770 |

*In this table, upper case letters represent sgRNA nucleotides that align to the exon sequence of the gene. Lower case letters represent sgRNA nucleotides that align to the intron sequence of the gene.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Generation of pLbCpf1-2A-GFP and pAsCpf1-2A-GFP Plasmids.

Human codon-optimized LbCpf1 and AsCpf1 were PCR amplified from pY016 plasmid (Zetsche et al., 2015) (pcDNA3.1-hLbCpf1), a gift from Feng Zhang (Addgene plasmid #69988) and pY010 plasmid (Zetsche et al., 2015) (pcDNA3.1-hAsCpf1), a gift from Feng Zhang (Addgene plasmid #69982), respectively. Cpf1 cDNA and T2A-GFP DNA fragment were cloned into the backbone of the pSpCas9(BB)-2A-GFP (PX458) plasmid (Ran et al., 2015), a gift from Feng Zhang (Addgene plasmid #48138) that was cut with AgeI/EcoRI to remove SpCas9(BB)-2A-GFP. In-Fusion HD cloning kit (Takara Bio) was used. Cpf1 guide RNAs (gRNAs) targeting the human DMD or the mouse Dmd locus were sub-cloned into a newly generated pLbCpf1-2A-GFP plasmid and pAsCpf1-2A-GFP plasmid using BbsI digestion and T4 ligation. Detailed primer sequences can be found in Table C, genomic target sequences can be found in Table D, and gRNA sequences can be found in Table E.

Human iPSC Maintenance, Nucleofection and Differentiation.

Human iPSCs were cultured in mTeSRTMI media (STEMCELL Technologies) and passaged approximately every 4 days (1:18 split ratio). One hour before nucleofection, iPSCs were treated with 10 μM ROCK inhibitor (Y-27632) and dissociated into single cells using Accutase (Innovative Cell Technologies, Inc.). 1×10⁶ iPSC cells were mixed with 5 pg of pLbCpf1-2A-GFP or pAsCpf1-2A-GFP plasmid and nucleofected using the P3 Primary Cell 4D-Nucleofector X kit (Lonza) according to manufacturer's protocol. After nucleofection, iPSCs were cultured in mTeSR™1 media supplemented with 10 μM ROCK inhibitor, penicillin-streptomycin (1:100) (ThermoFisher Scientific) and 100 pg/ml Primosin (InvivoGen). Three days post-nucleofection, GFP(+) and (−) cells were sorted by FACS and subjected to T7E1 assay. Single clones derived from GFP(+). iPSCs were picked and sequenced. iPSCs were induced to differentiate into cardiomyocytes, as previously described (Burridge et al. 2014).

Genomic DNA Isolation.

Genomic DNA of mouse 10T1/2 fibroblasts and human iPSCs was isolated using Quick-gDNA MiniPrep kit (Zymo Research) according to manufacturer's protocol.

RT-PCR.

RNA was isolated using TRIzol (ThermoFisher Scientific), according to manufacturer's protocol. cDNA was synthesized using iScript Reverse Transcription Supermix (Bio-Rad Laboratories) according to manufacturer's protocol. RT-PCR was performed using primers flanking DMD exon 47 and 52:

(SEQ ID NO: 1)
forward: 5′-CCCAGAAGAGCAAGATAAACTTGAA-3′;
(SEQ ID NO: 2)
reverse: 5′-CTCTGTTCCAAATCCTGCTTGT-3′

RT-PCR products amplified from WT cardiomyocytes, uncorrected cardiomyocytes and exon 51 skipped cardiomyocytes were 717 bps, 320 bps and 87 bps, respectively.

Dystrophin Western Blot Analysis.

Western blot analysis were performed as previously described (Long et al., 2014) using rabbit anti-dystrophin antibody (Abcam, ab15277) and mouse anti-cardiac myosin heavy chain antibody (Abcam, ab50967).

Dystrophin Immunocytochemistry and Immunohistochemistry.

iPSC-derived cardiomyocytes fixed with acetone, blocked with serum cocktail (2% normal horse serum/2% normal donkey serum/0.2% bovine serum albumin (BSA)/PBS), and incubated with dystrophin antibody (MANDYS8, 1:800, Sigma-Aldrich) and troponin-I antibody (H170, 1:200, Santa Cruz Biotechnology) in 0.2% BSA/PBS. Following overnight incubation at 4° C., they were incubated with secondary antibodies (biotinylated horse anti-mouse IgG, 1:200, Vector Laboratories, fluorescein-conjugated donkey anti-rabbit IgG, 1:50, Jackson Immunoresearch) for one-hour. Nuclei were counterstained with Hoechst 33342 (Molecular Probes).

Immunohistochemisty of skeletal muscle was performed as previously described (Long et al., 2014) using dystrophin antibody (MANDYS8, 1:800, Sigma-Aldrich). Nuclei were counterstained with propidium iodide (Molecular Probes).

Mitochondrial DNA Copy Number Quantification.

Genomic and mitochondrial DNA were isolated using Trizol, followed by back extraction as previously described (Zechner et al., 2010). KAPA SYBR FAST qPCR kit (Kapa Biosystems) was used to perform real-time PCR to quantitatively determine mitochondrial DNA copy number. Human mitochondrial ND1 gene was amplified using primers (forward: 5′-CGCCACATCTACCATCACCCTC-3′ (SEQ ID NO: 3); reverse: 5′-CGGCTAGGCTAGAGGTGGCTA-3′(SEQ ID NO: 4)). Human genomic LPL gene was amplified using primers (forward: 5′-GAGTATGCAGAAGCCCCGAGTC-3′ (SEQ ID NO: 5); reverse: 5′-TCAACATGCCCAACTGGTTTCTGG-3′ (SEQ ID NO: 6)). mtDNA copy number per diploid genome was calculated using formula:

ΔCT=(mtND1CT−LPL CT)

mtDNA copy number per diploid genome=2×2−ΔCT

Cellular Respiration Rates.

Oxygen consumption rates (OCR) were determined in human iPSC-derived cardiomyocytes using the XF24 Extracellular Flux Analyzer (Seahorse Bioscience) following the manufacturer's protocol as previously described (Baskin et al., 2014).

In Vitro Transcription of LbCpf1 mRNA and gRNA.

Human codon-optimized LbCpf1 was PCR amplified from pLbCpf1-2A-GFP to include the T7 promoter sequence (Table S1). The PCR product was transcribed using mMESSAGE mMACHINE T7 transcription kit (ThermoFisher Scientific) according to manufacturer's protocol. Synthesized LbCpf1 mRNA were poly-A tailed with E. coli Poly(A) Polymerase (New England Biolabs) and purified using NucAway spin columns (ThermoFisher Scientific).

The template for LbCpf1 gRNA in vitro transcription was PCR amplified from pLbCpf1-2A-GFP plasmid and purified using Wizard SV gel and PCR clean-up system (Promega). The LbCpf1 gRNA was synthesized using MEGAshortscript T7 transcription kit (ThermoFisher Scientific) according to manufacturer's protocol. Synthesized LbCpf1 gRNA were purified using NucAway spin columns (ThermoFisher Scientific).

Single-Stranded Oligodeoxynucleotide (ssODN).

ssODN was used as HDR template and synthesized by Integrated DNA Technologies as 4 nM Ultramer Oligonucleotides. ssODN was mixed with LbCpf1 mRNA and gRNA directly without purification. The sequence of ssODN is:

(SEQ ID NO: 7)
5′-TGATATGAATGAAACTCATCAAATATGCGTGTTAGTGTAAATGAACT
TCTATTTAATTTTGAGGCTCTGCAAAGTTCTTTAAAGGAGCAGCAGAATG
GCTTCAACTATCTGAGTGACACTGTGAAGGAGATGGCCAAGAAAGCACCT
TCAGAAATATGCCAGAAATATCTGTCAGAATTT-3′

CRISPR-Cpf1-Mediated Genome Editing by One-Cell Embryo Injection.

All animal procedures were approved by the Institutional Animal Care and Use Committee at the University of Texas Southwestern Medical Center. Injection procedures were performed as described previously (Long et al., 2014). The only modification was replacing Cas9 mRNA and Cas9 sgRNAs with LbCpf1 mRNA and LbCpf1 gRNAs.

PCR Amplification of Genomic DNA, T7E1 Assay, and TseI RFLP Analysis.

These methods were preformed as previously published (Long et al., 2014).

Statistical Analysis.

Statistical analysis was assessed by two-tailed Student's t-test. Data are shown as mean±SEM. A P<0.05 value was considered statistically significant.

### Example 2—Results

Correction of DMD iPSC-Derived Cardiomyocytes by Cpf1-Mediated Genome Editing.

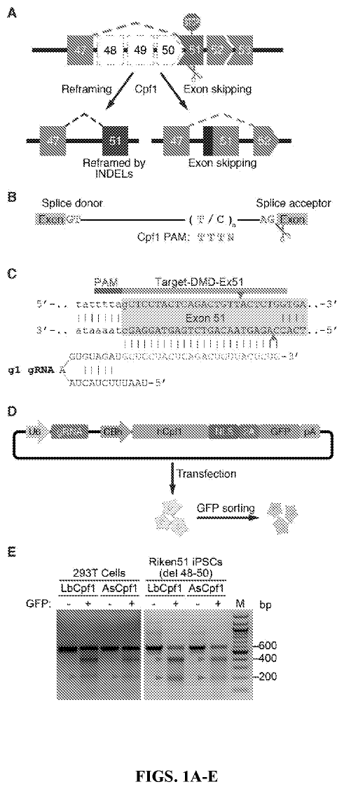

Exon deletions preceding exon 51 of the human DMD gene, which disrupt the open reading frame (ORF) by juxtaposing out of frame exons, represent the most common type of human DMD mutation (Aartsma-Rus et al., 2009). Skipping of exon 51 can, in principle, restore the DMD ORF in 13% of DMD patients with exon deletions (Cirak et al., 2011). To test the potential of Cpf1 to correct this type of “hot-spot” mutation, the inventors used DMD fibroblast-derived iPSCs (Riken HPS0164, abbreviated as Riken51), which harbor a deletion of exons 48 to 50, introducing a premature termination codon within exon 51 (FIG. 1A).

The splice acceptor region is generally T/C-rich (Padgett, 2012), which creates an ideal PAM sequence for genome editing by Cpf1 endonuclease (FIG. 1B). To rescue dystrophin expression in Riken51 iPSCs, the inventors used a Cpf1 gRNA to target exon 51, introducing small insertions and deletions (INDELs) in exon 51 by NHEJ and subsequently reframing the dystrophin ORF, theoretically, in one-third of corrected genes, a process inventors refer to as “reframing” (FIG. 1A). They also compared two Cpf1 orthologs, LbCpf1 (from Lachnospiraceae bacterium sp. ND2006; UniProt Accession No. AOA182DWE3; SEQ ID No. 443) and AsCpf1 (from Acidaminococcus sp. BV3L6; SEQ ID No. 442), which use the same PAM sequences for genome cleavage.

Cpf1 cleavage was targeted to the T-rich splice acceptor site of exon 51 using a guide RNA (designated g1) (FIG. 1C), which was cloned into plasmids pLbCpf1-2A-GFP and pAsCpf1-2A-GFP (FIG. 1D). These plasmids express human codon optimized LbCpf1 or AsCpf1, plus GFP; enabling fluorescence activated cell sorting (FACS) of Cpf1-expressing cells (FIG. 1D). Initially, inventors evaluated the cleavage efficiency of Cpf1-editing with g1 in human 293T cells. Both LbCpf1 and AsCpf1 efficiently induced DNA cleavage with g1, as detected using a T7E1 assay that recognizes and cleaves non-perfectly matched DNA (FIG. 1E).

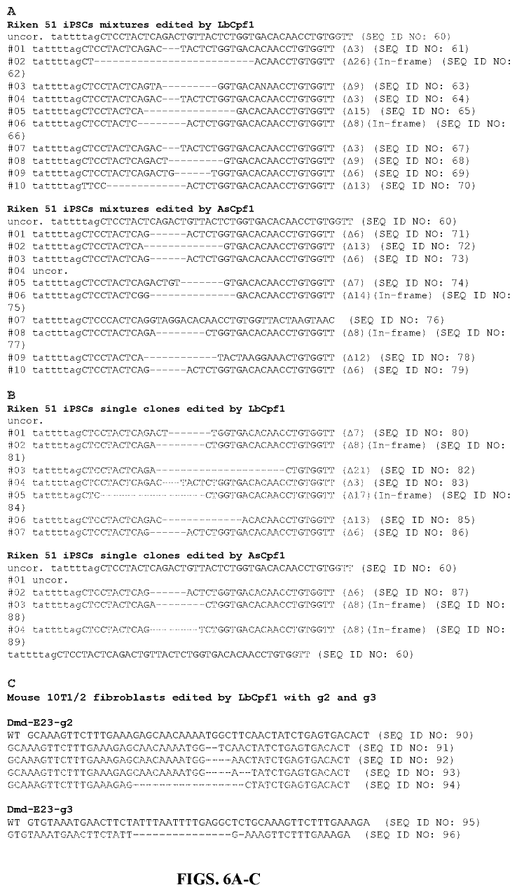

Next, inventors used LbCpf1 and AsCpf1 with g1 to edit Riken51 iPSCs, and by the T7E1 assay the inventors observed genome cleavage at DMD exon 51 (FIG. 1E). Genomic PCR products from the Cpf1-edited DMD exon 51 were cloned and sequenced (FIG. 6A). They observed INDELs near the exon 51 splice acceptor site in both LbCpf1- and AsCpf1-edited Riken51 iPSCs (FIG. 6A). Single clones from a mixture of reframed Riken51 iPSCs were picked and expanded and the edited genomic region was sequenced. Out of 12 clones, inventors observed four clones with reframed DMD exon 51, which restored the ORF (FIG. 6B).

Restoration of Dystrophin Expression in DMD iPSC-Derived Cardiomyocytes after Cpf1-Mediated Reframing.

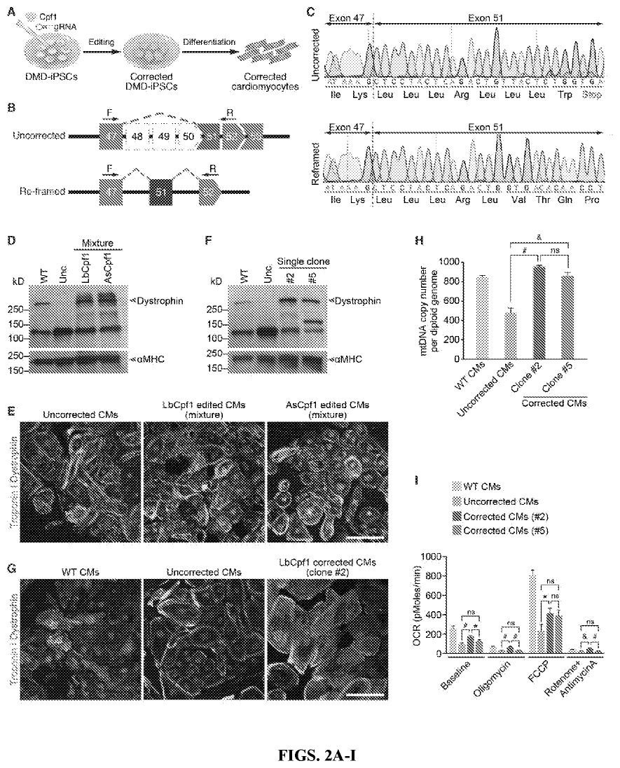

Riken51 iPSCs edited by CRISPR-Cpf1 using the reframing strategy were induced to differentiate into cardiomyocytes (Burridge et al., 2014) (FIG. 2A). Cardiomyocytes with the reframed DMD gene were identified by RT-PCR using a forward primer targeting exon 47 and a reverse primer targeting exon 52 and PCR products were sequenced (FIGS. 2B-C). Uncorrected iPSC-derived cardiomyocytes have a premature termination codon following the first 8 amino acids encoded by exon 51, which creates a premature stop codon (FIG. 2C). Cardiomyocytes differentiated from Cpf1-edited Riken51 iPSCs showed restoration of the DMD ORF as seen by sequencing of the RT-PCR products from amplification of exons 47 to 52 (FIG. 2C). The inventors also confirmed restoration of dystrophin protein expression by Western blot analysis and immunocytochemistry using dystrophin antibody (FIGS. 2D-E). Surprisingly, even without clonal selection and expansion, cardiomyocytes differentiated from Cpf1-edited iPSC mixtures showed levels of dystrophin protein comparable to WT cardiomyocytes (FIG. 2D).

From mixtures of LbCpf1-edited Riken51 iPSCs, the inventors picked two clones (clone #2 and #5) with in-frame INDELs of different sizes and differentiated the clones into cardiomyocytes. Clone #2 had an 8 bp deletion at the 5′-end of exon 51, together with an endogenous deletion of exons 48-50. The total 405 bp deletion restored the DMD ORF and allowed for the production of a truncated dystrophin protein with a 135 amino acid deletion. Clone #5 had a 17 bp deletion in exon 51 and produced dystrophin protein with a 138 amino acid deletion. Although there is high efficiency of cleavage by Cpf1, the amount of DNA inserted or deleted at the cleavage site varies. Additionally, INDELs can generate extra codons at the edited locus, causing changes of the ORF. The dystrophin protein expressed by clone #2 cardiomyocytes generated an additional four amino acids (Leu-Leu-Leu-Arg) between exon 47 and exon 51, whereas dystrophin protein expressed by clone #5 cardiomyocytes generated only one additional amino acid (Leu). From both clones #2 and #5, the inventors observed restored dystrophin protein by Western blot analysis and immunocytochemistry (FIGS. 2F-G). Due to the large size of dystrophin, the internally-deleted forms migrated similarly to WT dystrophin on SDS-PAGE.

The inventors also performed functional analysis of DMD iPSC-derived cardiomyocytes by measuring mitochondrial DNA copy number and cellular respiration rates. Uncorrected DMD iPSC-derived cardiomyocytes had significantly fewer mitochondria than the LbCpf1-corrected cardiomyocytes (FIG. 2H). After LbCpf1-mediated reframing, both corrected clones restored mitochondrial number to a level comparable to that of WT cardiomyocytes (FIG. 2H). Clone #2 iPSC-derived cardiomyocytes also showed an increase in oxygen consumption rate (OCR) compared to uncorrected iPSC-derived cardiomyocytes at baseline (FIG. 2I). OCR was inhibited by oligomycin in all iPSC-derived cardiomyocytes, and treatment with the uncoupling agent FCCP enhanced OCR. Finally, treatment with rotenone and antimycin A further inhibited OCR in all cardiomyocytes. These results demonstrate that Cpf1-mediated DMD correction improved respiratory capacity of mitochondria in corrected iPSC-cardiomyocytes. Our findings show that Cpf1-mediated reframing is a highly efficient strategy to rescue DMD phenotypes in human cardiomyocytes.

Restoration of Dystrophin Expression in DMD iPSC-Derived Cardiomyocytes by Cpf1-Mediated Exon Skipping.

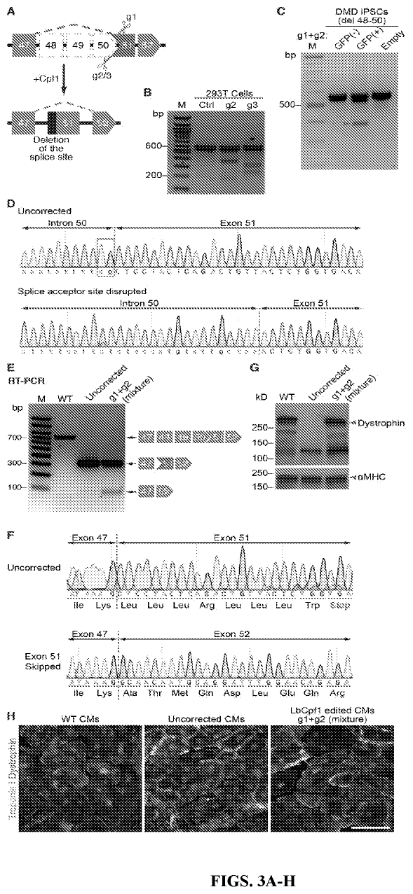

In contrast to the single gRNA-mediated reframing method, which introduces small INDELs, exon skipping uses two gRNAs to disrupt splice sites and generates a large deletion (FIG. 3A). As an independent strategy to restore dystrophin expression in the Riken51 iPSCs, the inventors designed two LbCpf1 gRNAs (g2 and g3) that target the 3′-end of intron 50 and tested the cleavage efficiency in human 293T cells. T7E1 assay showed that g2 had higher cleavage efficiency within intron 50 compared to g3 (FIG. 3B). Therefore, the inventors co-delivered LbCpf1, g2 and g1 (g1 targets the 5′ region of exon 51) into Riken51 iPSCs with the aim of disrupting the splice acceptor site of exon 51. Genomic PCR showed a lower band in LbCpf1-edited iPSCs (FIG. 3C) and sequencing data confirmed the presence of a deletion of −200 bp between intron 50 and exon 51, which disrupted the conserved splice acceptor site (FIG. 3D).

Riken51 iPSCs edited by the exon skipping strategy with g1 and g2 were differentiated into cardiomyocytes. Cells harboring the edited DMD allele were identified by RT-PCR using a forward primer targeting exon 47 and a reverse primer targeting exon 52; showing deletion of the exon 51 splice acceptor site which allows skipping of exon 51 (FIG. 3E). Sequencing of the RT-PCR products confirmed that exon 47 was spliced to exon 52, which restored the DMD ORF (FIG. 3F). Western blot analysis and immunocytochemistry confirmed the restoration of dystrophin protein expression in a mixture of LbCpf1-edited cardiomyocytes with g1 and g2 (FIGS. 3G-H). Thus, Cpf1-editing by the exon skipping strategy is highly efficient in rescuing the DMD phenotype in human cardiomyocytes.

Restoration of Dystrophin in Mdx Mice by Cpf1-Mediated Correction.

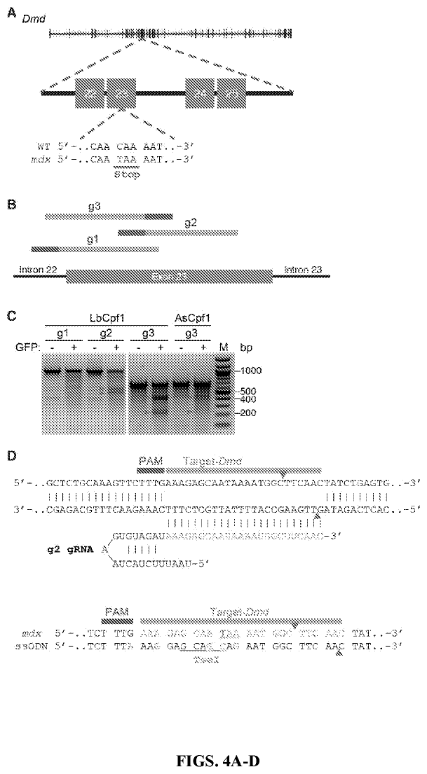

To further evaluate the potential of Cpf1-mediated Dmd correction in vivo, the inventors used LbCpf1 to permanently correct the mutation in germline of mdx mice by HDR-mediated correction or NHEJ-mediated reframing. mdx mice carry a nonsense mutation in exon 23 of the Dmd gene, due to a C to T transition (FIG. 4A). Three gRNAs (g1, g2 and g3) that target exon 23 were screened and tested in mouse 10T1/2 fibroblasts for cleavage efficiency (FIG. 4B). The T7E1 assay revealed that LbCpf1 and AsCpf1 had different cleavage efficiencies at Dmd exon 23 (FIG. 4C). Based on sequencing results, LbCpf1-mediated genome editing using g2 generated a greater occurrence of INDELs in mouse fibroblasts compared to g3 (FIG. 6C).

LbCpf1-editing with g2 recognizes a PAM sequence 9 bps upstream of the mutation site and creates a staggered double-stranded DNA cut 8 bps downstream of the mutation site (FIG. 4D). To obtain HDR genome editing, the inventors used a 180 bp single-stranded oligodeoxynucleotide (ssODN) in combination with LbCpf1 and g2 since it has been shown that ssODNs are more efficient in introducing genomic modification than double-stranded donor plasmids (Wu et al., 2013; Long et al., 2014). They generated a ssODN containing 90 bp of homology sequence flanking the cleavage site, including, four silent mutations and a TseI restriction site to facilitate genotyping as previously described (Long et al., 2014). This ssODN was designed to be used with LbCpf1 and g2 to correct the C to T mutation within Dmd exon 23 and to restore dystrophin in mdx mice by HDR.

Correction of Muscular Dystrophy in Mdx Mice by LbCpf1-Mediated HDR.

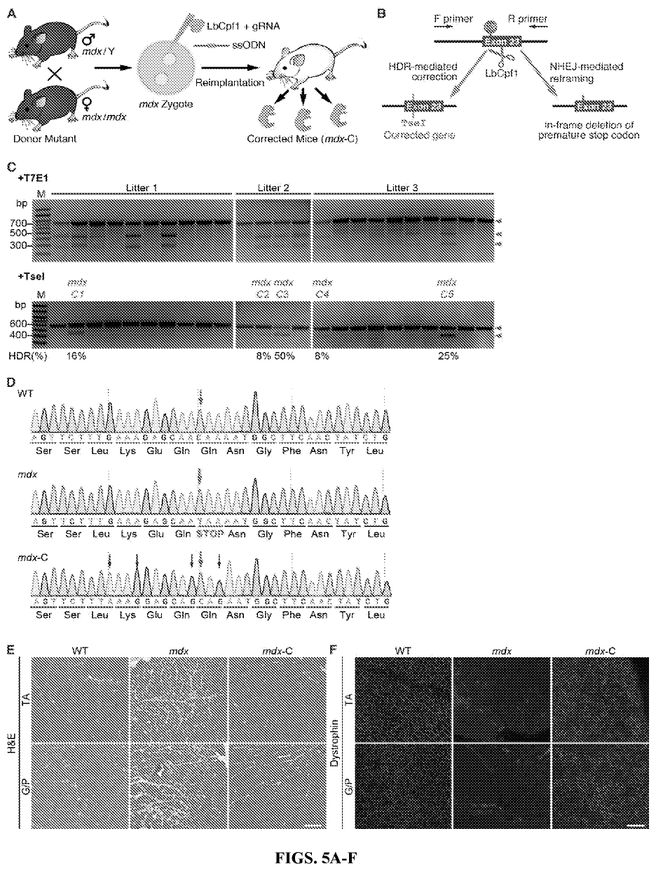

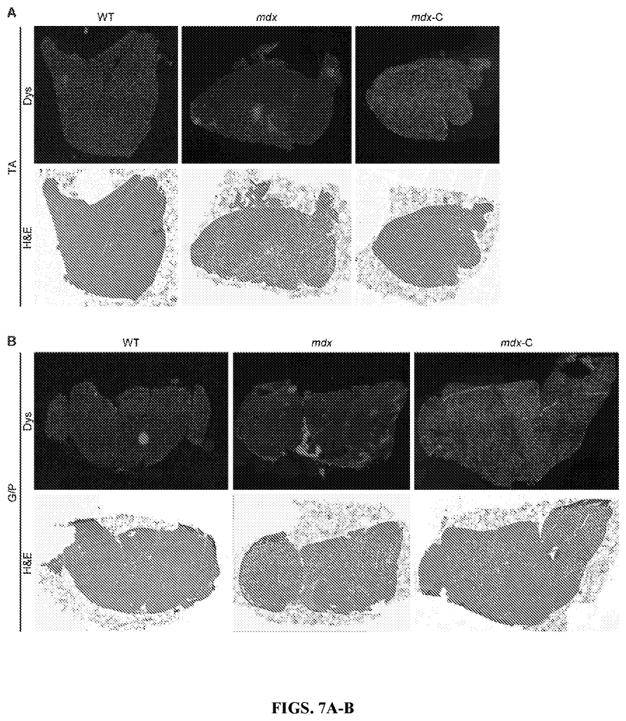

mdx zygotes were co-injected with in vitro transcribed LbCpf1 mRNA, in vitro transcribed g2 gRNA and 180 bp ssODN and re-implanted into pseudo-pregnant females (FIG. 5A). Three litters of LbCpf1-edited mdx mice were analyzed by T7E1 assay and TseI RFLP (restriction fragment length polymorphism) (FIGS. 5B-C). Out of 24 pups born, 12 were T7E1 positive and 5 carried corrected alleles (mdx C1-C5), as detected by TseI RFLP and sequencing (FIGS. 5C-D). Skeletal muscles (tibialis anterior and gastrocnemius-plantaris) from WT, mdx and LbCpf1-corrected mdx-C mice were analyzed at 4 weeks of age. Hematoxylin and eosin (H&E) staining of muscle showed fibrosis and inflammatory infiltration in mdx muscle, whereas LbCpf1-corrected (mdx-C) muscle displayed normal muscle morphology and no signs of a dystrophic phenotype (FIG. 5E and FIGS. 7A-B). Immunohistochemistry showed absence of dystrophin-positive fibers in muscle sections of mdx mice, whereas mdx-C muscle corrected by LbCpf1-mediated HDR showed dystrophin protein expression in a majority of muscle fibers (FIG. 5F and FIGS. 7A-B). These findings show that LbCpf1-mediated editing of germline DNA can effectively prevent muscular dystrophy in mice.

### Example 3—Discussion

In this study, the inventors show that the newly discovered CRISPR-Cpf1 nuclease can efficiently correct DMD mutations in patient-derived iPSCs and mdx mice, allowing for restoration of dystrophin expression. Lack of dystrophin in DMD has been show to disrupt integrity of the sarcolemma, causing mitochondria dysfunction and oxidative stress (Millay et al., 2008; Mourkioti et al., 2013). They found increased mitochondrial DNA and higher oxygen consumption rates in LbCpf1-corrected iPSC-derived cardiomyocytes compared to uncorrected DMD iPSC-derived cardiomyocytes. Metabolic abnormalities of human DMD iPSC-derived cardiomyocytes were also rescued by Cpf1-mediated genomic editing. The inventors' findings also demonstrated the robustness and efficiency of Cpf1 in mouse genome editing. By using HDR-mediated correction, the ORF of the mouse Dmd gene was completely restored and pathophysiological hallmarks of the dystrophic phenotype such as fibrosis and inflammatory infiltration were also rescued.

Two different strategies—“reframing” and “exon skipping”—were applied to restore the ORF of the DMD gene using LbCpf1-mediated genome editing. Reframing creates small INDELs and restores the ORF by placing out-of-frame codons in-frame. Only one gRNA is required for reframing. Although the inventors did not observe any differences in subcellular localization between WT dystrophin protein and reframed dystrophin protein by immunocytochemistry, they observed differences in dystrophin expression level, mitochondrial DNA quantity, and oxygen consumption rate in separate edited clones, suggesting that reframed dystrophin may not be structurally or functionally identical to WT dystrophin.

Various issues should be considered with respect to the use of one or two gRNAs with Cpf1-editing. Here, the inventors show that two gRNAs are more effective than one gRNA for disruption of the splice acceptor site compared to reframing. When using two gRNAs, Cpf1-editing excises the intervening region and not only removes the splice acceptor site but can be designed to remove deleterious “AG” nucleotides, eliminating the possibility of generating a pseudo-splice acceptor site. However, with two gRNAs there is the necessity that both gRNAs cleave simultaneously, which may not occur. If only one of the two gRNAs cleaves, the desired deletion will not be generated. However, there remains the possibility that cleavage with one of the two gRNAs will generate INDELS at the targeted exon region, reframing the ORF, since in theory, one third of the INDELS will be in-frame. Using one gRNA to disrupt the splice acceptor site seems more efficient because it eliminates the need for two simultaneous cuts to occur. However, there is uncertainty with respect to the length of the INDEL generated by one gRNA-mediated editing. More importantly, with one gRNA there remains the possibility of leaving exonic “AG” nucleotides near the cleavage site, which can serve as an alternative pseudo-splice acceptor site.

With its unique T-rich PAM sequence, Cpf1 further expands the genome editing range of the CRISPR family, which is important for potential correction of other disease-related mutations since not all mutation sites contain G-rich PAM sequences for SpCas9 or PAMs for other Cas9 orthologues. Moreover, the staggered cut generated by Cpf1 may be also advantageous for NHEJ-mediated genome editing (Maresca et al., 2013). Finally, the LbCpf1 used in this study is 140-amino-acids smaller than the most widely used SpCas9, which would enhance packaging and delivery by AAV. To evaluate the targeting specificity of Cpf1, two groups (Kim et al., 2016; Tsai et al., 2016) determined the genome-wide editing efficiency of LbCpf1 and AsCpf1 by multiple methods. Both studies showed that LbCpf1 and AsCpf1 had high genome-wide targeting efficiency comparable to that of SpCas9 and high targeting specificity because LbCpf1 and AsCpf1 cannot tolerate mismatches at the 5′ PAM proximal region, lessening the frequency of off-targeting effect.

These findings show that Cpf1 is highly efficient in correcting human DMD and mouse Dmd mutations in vitro and in vivo. CRISPR-Cpf1-mediated genome editing represents a new and powerful approach to permanently eliminate genetic mutations and rescue abnormalities associated with DMD and other disorders.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Angel et al., Mol. Cell. Biol., 7:2256, 1987a.
Angel et al., Cell, 49:729, 1987b.
Aartsma-Rus et al., Hum. Mutat. 30, 293-299, 2009.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Baskin et al., EMBO Mol Med 6, 1610-1621, 2014.
Benvenisty and Neshif, Proc. Natl. Acad. Sci. USA, 83:9551-9555, 1986.
Berkhout et al., Cell, 59:273-282, 1989.
Bhavsar et al., Genomics, 35(1):11-23, 1996.
Bikard et al., Nucleic Acids Res. 41(15): 7429-7437, 2013.
Blanar et al., EMBO J., 8:1139, 1989.
Bodine and Ley, EMBO J., 6:2997, 1987.
Boshart et al., Cell, 41:521, 1985.
Bostick et al., Mol Ther 19, 1826-1832, 2011.
Bosze et al., EMBO J., 5(7):1615-1623, 1986.
Braddock et al., Cell, 58:269, 1989.
Bulla and Siddiqui, J. Virol., 62:1437, 1986.
Burridge et al., Nat. Methods 11, 855-860, 2014.
Bushby et al., Lancet Neurol., 9(1): 77-93 (2010).
Bushby et al., Lancet Neurol., 9(2): 177-198 (2010).
Campbell & Kahl, Nature 338, 259-262, 1989.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chang et al., Stem Cells., 27:1042-1049, 2009.
Chatterjee et al., Proc. Natl. Acad. Sci. USA, 86:9114, 1989.
Chen and Okayama, Mol. Cell Biol., 7:2745-2752, 1987.
Cho et al., Nat. Biotechnol. 31(3): 230-232, 2013.
Choi et al., Cell, 53:519, 1988.
Cirak et al., The Lancet 378, 595-605, 2011.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
DeWitt et al., Sci Transl Med 8, 360ra134-360ra134, 2016.
Donnelly et al., J. Gen. Virol. 82, 1027-1041, 2001.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
EP 0273085
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Foecking and Hofstetter, Gene, 45(1):101-105, 1986.
Fonfara et al., Nature 532, 517-521, 2016.
Fraley et al., Proc Natl. Acad. Sci. USA, 76:3348-3352, 1979 Franz et al., Cardoscience, 5(4):235-43, 1994.
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goncalves et al., Mol Ther, 19(7): 1331-1341 (2011).
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hirochika et al., J. Virol., 61:2599, 1987.
Holbrook et al., Virology, 157:211, 1987.
Hollinger & Chamberlain, Current Opinion in Neurology 28, 522-527, 2015.
Horlick and Benfield, Mol. Cell. Biol., 9:2396, 1989.
Horwich et al., J. Virol., 64:642-650, 1990.
Hsu et al., Natl Biotechnol. 31:827-832, 2013
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jinek et al., Science 337, 816-821, 2012.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J Biol Chem., 266(6):3361-3364, 1991.
Kawamoto et al., Mol. Cell. Biol., 8:267, 1988.
Kelly et al., J. Cell Biol., 129(2):383-96, 1995.
Kiledjian et al., Mol. Cell. Biol., 8:145, 1988.
Kim et al., Nature Biotechnology, 1-2, 2016.
Kim et al., Nature Biotechnology 34, 876-881, 2016.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., Hypertension, 27(3):715-22, 1996
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Levinson et al., Nature, 295:79, 1982.
Levrero et al., Gene, 101:195-202, 1991.
Lin et al., Mol. Cell. Biol., 10:850, 1990.
Long et al., Science 345:1184-1188, 2014.
Long et al., Genome Editing of Monogenic Neuromuscular Diseases: A Systematic Review. JAMA Neurol, doi:10.1001/jamaneurol.2016.3388, 2016.
Long et al., Science 351, 400-403, 2016.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mali et al., Science 339, 823-826, 2013a.
Mali et al., Nat Methods 10, 957-963, 2013b.
Mali et al., Nat. Biotechnol. 31:833-838, 2013c.
Mann et al., Cell, 33:153-159, 1983.
Maresca et al., Genome Research 23, 539-546, 2013.
Markowitz et al., J. Virol., 62:1120-1124, 1988.
Martari, M., et al. Hum Gene Ther, 20(7):759-766 (2009).
McNeall et al., Gene, 76:81, 1989.
Miksicek et al., Cell, 46:203, 1986.
Millay et al., Nat. Med. 14, 442-447, 2008.
Mojica et al., J. Mol. Evol. 60, 174-182, 2005.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Mourkioti et al., Nature Cell Biology 15, 895-904, 2013.
Muesing et al., Cell, 48:691, 1987.
Nelson et al., Science 351, 403-407, 2016.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Padgett, R. A., Trends Genet. 28, 147-154, 2012.
Palmiter et al., Cell, 29:701, 1982a.
Palmiter et al., Nature, 300:611, 1982b.
Paskind et al., Virology, 67:242-248, 1975.
Pech et al., Mol. Cell. Biol., 9:396, 1989.
Perales et al., Proc. Natl. Acad. Sci. USA, 91(9):4086-4090, 1994.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Ran et al., Nature 520, 186-191, 2015.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Shimizu-Motohashi et al., Am J Transl Res 8, 2471-2489, 2016.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Tabebordbar et al., Science 351, 407-411, 2016.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Tóth et al., Biology Direct, 1-14, 2016.
Tronche et al., Mol. Biol. Med., 7:173, 1990.
Trudel and Constantini, Genes and Dev. 6:954, 1987.
Tsai et al., Nature Biotechnology 34, 882-887, 2016.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Wagner et al., Proc. Natl. Acad. Sci. USA, 87(9):3410-3414, 1990.
Wang et al., Cell, 153:910-910, 2013.
Weber et al., Cell, 36:983, 1984.
Weinberger et al. Mol. Cell. Biol., 8:988, 1984.
Winoto and Baltimore, Cell, 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Cell Stem Cell 13, 659-662, 2013.
Wu et al., Nat Biotechnol 32, 670-676, 2014.
Xu et al., Mol Ther 24, 564-569, 2016.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yin et al., Nat Biotechnol 32, 551-553, 2014.
Yin et al., Physiol Rev 93, 23-67, 2013.
Young et al., Cell Stem Cell 18, 533-540, 2016.
Yutzey et al. Mol. Cell. Biol., 9:1397, 1989.
Zechner et al., Cell Metabolism 12, 633-642, 2010.
Zelenin et al., FEBS Lett., 280:94-96, 1991.
Zetsche et al., Cell 163, 759-771, 2015.
Ziober and Kramer, J. Bio. Chem., 271(37):22915-22922, 1996.

1. A composition comprising a sequence encoding a Cpf1 polypeptide and a sequence encoding a DMD guide RNA (gRNA), wherein the DMD gRNA targets a dystrophin splice site, and wherein the DMD gRNA comprises any one of SEQ ID No. 448 to 770.

2. The composition of claim 1, wherein the sequence encoding the Cpf1 polypeptide is isolated or derived from a sequence encoding a Lachnospiraceae Cpf1 polypeptide or an Acidaminococcus Cpf1 polypeptide.

3-7. (canceled)

8. The composition of claim 1, wherein a first vector comprises the sequence encoding the Cpf1 polypeptide and a second vector comprises the sequence encoding the DMD gRNA.

9. The composition of claim 8, wherein the first vector or the sequence encoding the Cpf1 polypeptide further comprises a first polyA sequence.

10. The composition of claim 8, wherein the second vector or the sequence encoding the DMD gRNA further comprises a second polyA sequence.

11. The composition of claim 8, wherein the first vector or the sequence encoding the Cpf1 polypeptide further comprises a first promoter sequence.

12. The composition of claim 8, wherein the second vector or the sequence encoding the DMD gRNA further comprises a second promoter sequence.

13. The composition of claim 11, wherein the first promoter sequence and the second promoter sequence are identical.

14. The composition of claim 11, wherein the first promoter sequence and the second promoter sequence are not identical.

15-16. (canceled)

17. The composition of claim 11, wherein the first promoter sequence or the second promoter sequence comprises a muscle-cell specific promoter.

18. The composition of claim 17, wherein the muscle-cell specific promoter is a myosin light chain-2 promoter, an α-actin promoter, a troponin 1 promoter, a Na⁺/Ca²⁺ exchanger promoter, a dystrophin promoter, an α7 integrin promoter, a brain natriuretic peptide promoter, an αB-crystallin/small heat shock protein promoter, an α-myosin heavy chain promoter, or an ANF promoter.

19-37. (canceled)

38. The composition of claim 1, wherein the composition comprises a sequence codon optimized for expression in a mammalian cell.

39. The composition of claim 38, wherein the composition comprises a sequence codon optimized for expression in a human cell.

40. The composition of claim 39, wherein the sequence encoding the Cpf1 polypeptide is codon optimized for expression in human cells.

41. The composition of claim 1, wherein the splice site is a splice donor site.

42. The composition of claim 1, wherein the splice site is a splice acceptor site.

43-48. (canceled)

49. The composition of claim 8, wherein the first vector or the second vector is a viral vector.

50. (canceled)

51. The composition of claim 49, wherein the viral vector is an adeno-associated viral (AAV) vector.

52. The composition of claim 51, wherein the AAV vector is replication-defective or conditionally replication defective.

53. The composition of claim 51, wherein the AAV vector is a recombinant AAV vector.

54. The composition of claim 51, wherein the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

55. (canceled)

56. The composition of claim 1, further comprising a pharmaceutically acceptable carrier.

57. A cell comprising the composition of claim 1.

58. The cell of claim 57, wherein the cell is a muscle cell, a satellite cell or a precursor thereof.

59. The cell of claim 57, wherein the cell is an iPSC or an iCM.

60. A composition comprising the cell of claim 57.

61. A method of correcting a dystrophin gene defect comprising contacting a cell and a composition of claim 1 under conditions suitable for expression of the Cpf1 polypeptide and the gRNA, wherein the Cpf1 polypeptide disrupts the dystrophin splice site; and wherein disruption of the splice site results in selective skipping of a mutant DMD exon.

62. The method of claim 61, wherein the mutant DMD exon is exon 23.

63. The method of claim 61, wherein the mutant DMD exon is exon 51.

64. The method of claim 61, wherein the cell is in vivo, ex vivo, in vitro or in situ.

65. A cell produced by the method of claim 61.

66. A composition comprising the cell of claim 65.

67. A method of treating muscular dystrophy in a subject in need thereof comprising administering to the subject a therapeutically effective amount of a composition of claim 1.

68. The method of claim 67, wherein the composition is administered locally.

69. (canceled)

70. The method of claim 68, wherein the composition is administered to a muscle tissue by intramuscular infusion or injection.

71. The method of claim 70, wherein the muscle tissue comprises a tibialis anterior tissue, a quadricep tissue, a soleus tissue, a diaphragm tissue or a heart tissue.

72. The method of claim 67, wherein the composition is administered systemically, such as by intravenous infusion or injection.

73-79. (canceled)

80. The method of claim 67, wherein the subject is a neonate, an infant, a child, a young adult, or an adult.

81. The method of claim 67, wherein the subject has muscular dystrophy.

82. (canceled)

83. The method of claim 67, wherein the subject is male.

84-96. (canceled)

97. The method of claim 67, wherein the subject is less than 10 years old.

98. (canceled)

99. The method of claim 97, wherein the subject is less than 2 years old.

100-102. (canceled)
